(19) United States
(12) Patent Application Publication
(10) Pub. No.: US 2009/0312406 A1
(43) Pub. Date: Dec. 17, 2009

(54) COUMARIN COMPOUNDS AND THEIR USE FOR TREATING VIRAL INFECTION

(76) Inventors: Hsing-Pang Hsieh, Taipei (TW); Tsu-An Hsu, Taipei (TW); Jiann-Yih Yeh, Kaohsiung City (TW); Jim-Tong Horng, Taoyuan (TW); Shin-Ru Shih, Taoyuan (TW); Sui-yuan Chang, Taipei (TW); Yu-Sheng Chao, Monmouth Junction, NJ (US)

Correspondence Address: OCCHIUTI ROHLICEK & TSAO, LLP, 10 FAWCETT STREET, CAMBRIDGE, MA, 02138, US

(21) Appl. No.: 12/481,789

(22) Filed: Jun. 10, 2009

Related U.S. Application Data
(60) Provisional application No. 61/060,927, filed on Jun. 12, 2008.

(51) Int. Cl. A61K 31/352; A61P 31/12

(52) U.S. Cl. 514/455

(57) ABSTRACT

A method for treating an infection with a virus. The method includes administering to a subject in need thereof an effective amount of one or more coumarin compounds of formula (I):

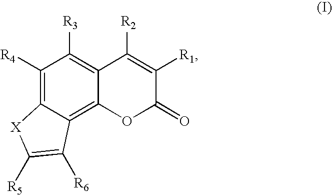

wherein R₁, R₂, R₃, R₄, R₅, R₆, and X are defined herein. Also disclosed is a pharmaceutical composition including a coumarin compound.

### CROSS-REFERENCE TO RELATED APPLICATION

This application claims the benefit of the priority pursuant to 35 U.S.C. § 119(e) of U.S. Provisional Patent Application No. 61/060,927, filed Jun. 12, 2008. The content of the prior application is incorporated herein by its entirety.

### BACKGROUND

There are a wide variety of viruses that cause various disorders, ranging from common human ailments (e.g., common cold, flu, chickenpox, and cold sore) to serious human diseases (e.g., Ebola, avian influenza, AIDS, and SARS). Some viruses are established causes of malignancy in humans and other animals. For example, papillomavirus, hepatitis B and hepatitis C virus, Epstein-Barr virus, and human T-lymphotropic virus have been associated with human cancers.

One of the most effective treatments of viral diseases is use of antiviral drugs. Different antiviral drugs target different stages of the viral life cycle. Taking influenza treatment for example, conventional anti-influenza drugs inhibit the membrane fusion or replication step by targeting viral hemagglutinin, neuraminidase, M2 ion channel, or 3P polymerase complex, or host factors such as kinases, as described in, e.g., Hsieh et al., Current Pharmaceutical Design, 2007, 13, 3531-3542.

Coumarin compounds, a binding ligand of nucleic acid, have been studied for their therapeutic use.

### SUMMARY

This invention is based on the discovery that certain coumarin compounds have potent anti-virus activity. Thus, this invention relates to coumarin compounds and to their uses in the treatment of an infection with a virus, especially influenza virus.

In one aspect, this invention features treating an infection with a virus by administering to a subject in need of the treatment an effective amount to a coumarin compound of formula (I):

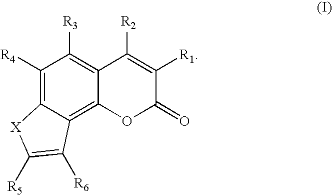

In formula (I), each of R₁, R₂, R₃, and R₄, independently, is H, alkyl, alkenyl, alkynyl, cycloalkyl, cycloalkenyl, heterocycloalkyl, heterocycloalkenyl, aryl, heteroaryl, halo, nitro, cyano, amino, hydroxy, alkoxy, aryloxy, C(O)Rₐ, C(O)ORₐ, C(O)NRₐR_{b}, C(S)R_{b}, or C(NR_{b})Rₐ, in which each of Rₐ and R_{b}, independently, is H, alkyl, alkenyl, alkynyl, hydroxy, alkoxy, amino, cycloalkyl, cycloalkenyl, heterocycloalkyl, heterocycloalkenyl, aryl, or heteroaryl; or R₁ and R₂, together with the carbon atoms to which they are bonded, are cycloalkenyl, heterocycloalkenyl, aryl, or heteroaryl; or R₂ and R₃, together with the carbon atoms to which they are bonded, are cycloalkenyl or heterocycloalkenyl; or R₃ and R₄, together with the carbon atoms to which they are bonded, are cycloalkenyl, heterocycloalkenyl, aryl, or heteroaryl; R₅ is alkyl substituted with aryl or hydroxy, alkenyl, alkynyl, cycloalkyl, cycloalkenyl, heterocycloalkyl, heterocycloalkenyl, aryl, heteroaryl, halo, nitro, cyano, amino, hydroxy, alkoxy, aryloxy, C(O)R_{c}, C(O)OR_{c}, C(O)NR_{c}R_{d}, C(S)R_{d}, or C(NR_{d})R_{c}, in which each of R_{c} and R_{d}, independently, is H, alkyl, alkenyl, alkynyl, hydroxy, alkoxy, amino, cycloalkyl, cycloalkenyl, heterocycloalkyl, heterocycloalkenyl, aryl, or heteroaryl; R₆ is H, alkyl, alkenyl, alkynyl, cycloalkyl, cycloalkenyl, heterocycloalkyl, heterocycloalkenyl, aryl, heteroaryl, halo, nitro, cyano, amino, hydroxy, alkoxy, aryloxy, C(O)R_{c}, C(O)OR_{c}, C(O)NR_{c}R_{d}, C(S)R_{d}, or C(NR_{d})R_{c}; or R₅ and R₆, together with the carbon atoms to which they are bonded, are cycloalkenyl, heterocycloalkenyl, aryl, or heteroaryl; and X is O, S, or N(Rₑ), in which Rₑ is H, alkyl, cycloalkyl, heterocycloalkyl, aryl, or heteroaryl. Examples of the virus include, but are not limited to, influenza virus, human rhinovirus 2, Herpes simplex virus, enterovirus 71 (EV 71), Coxsackie Virus B3, Hepatitis C virus, Hepatitis B virus, Epstein-Barr virus (EBV), and Human Immunodeficiency Virus.

In particular, this invention features a method for treating influenza virus infection, by administering to a subject in need thereof an effective amount of a compound of formula (I) shown above. Referring to formula (I), a subset of the just-described compounds are those in which R₅ is alkyl substituted with aryl or hydroxy, cycloalkyl, aryl, halo, C(O)R_{c}, or C(O)OR_{c}. In these compounds, R₅ can be alkyl substituted with aryl or C(O)R_{c}, in which R_{c} can be aryl or heteroaryl; R₆ can be alkyl, cycloalkyl, aryl, or heteroaryl; each of R₁, R₂, R₃, and R₄, independently, can be H, alkyl, aryl, heteroaryl, nitro, hydroxy, alkoxy, aryloxy, or C(O)Rₐ, or R₁ and R₂, together with the carbon atoms to which they are bonded, can be cycloalkenyl, heterocycloalkenyl, aryl, or heteroaryl; R₂ can be alkyl; or X can be O.

Another subset of the coumarin compounds of formula (I), for treating viral infection, includes those in which R₅ is C(S)R_{d} or C(NR_{d})R_{c}. In these compounds, R₆ can be alkyl, cycloalkyl, aryl, or heteroaryl; each of R₁, R₂, R₃, and R₄, independently, can be H, alkyl, aryl, heteroaryl, nitro, hydroxy, alkoxy, aryloxy, or C(O)Rₐ, or R₁ and R₂, together with the carbon atoms to which they are bonded, can be cycloalkenyl, heterocycloalkenyl, aryl, or heteroaryl; R₂ can be alkyl; or X can be O.

Yet another subset of the above-described coumarin compounds includes those in which R₆ is alkyl, cycloalkyl, aryl, or heteroaryl. In these compounds, R₅ can be alkyl substituted with aryl or C(O)R_{c}, in which R_{c} can be aryl or heteroaryl; R₆ can be aryl or heteroaryl; each of R₁, R₂, R₃, and R₄, independently, can be H, alkyl, aryl, heteroaryl, nitro, hydroxy, alkoxy, aryloxy, or C(O)Rₐ, or R₁ and R₂, together with the carbon atoms to which they are bonded, can be cycloalkenyl, heterocycloalkenyl, aryl, or heteroaryl; R₂ can be alkyl; or X can be O.

Still two other subsets of these coumarin compounds include those in which X is O and those in which each of R₁, R₂, R₃, and R₄, independently, is H, alkyl, aryl, heteroaryl, nitro, hydroxy, alkoxy, aryloxy, or C(O)Rₐ, or R₁ and R₂, together with the carbon atoms to which they are bonded, are cycloalkenyl, heterocycloalkenyl, aryl, or heteroaryl.

The term “treating” or “treatment” refers to administering one or more coumarin compounds to a subject, who has a viral infection, a symptom of or a predisposition toward it, with the purpose to confer a therapeutic effect, e.g., to cure, relieve, alter, affect, ameliorate, or prevent the infection, the symptom of or the predisposition toward it. Such a subject can be identified by a health care professional based on results from any suitable diagnostic method. “An effective amount” refers to the amount of one or more active coumarin compounds that is required to confer a therapeutic effect on a treated subject. Effective amounts may vary, as recognized by those skilled in the art, depending on route of administration, excipient usage, and the possibility of co-usage with other agents.

The term “alkyl” refers to a straight or branched monovalent hydrocarbon containing, unless otherwise stated, 1-20 carbon atoms (e.g., C₁-C₁₀). Examples of alkyl include, but are not limited to, methyl, ethyl, n-propyl, i-propyl, n-butyl, i-butyl, and t-butyl. The term “alkylene” refers to a straight or branched bivalent hydrocarbon, containing 1-20 carbon atoms (e.g., C₁-C₁₀). Examples of alkylene include, but are not limited to, methylene and ethylene. The term “alkenyl” refers to a straight or branched monovalent or bivalent hydrocarbon containing 2-20 carbon atoms (e.g., C₂-C₁₀) and one or more double bonds. Examples of alkenyl include, but are not limited to, ethenyl, propenyl, propenylene, allyl, and 1,4-butadienyl. The term “alkynyl” refers to a straight or branched monovalent or bivalent hydrocarbon containing 2-20 carbon atoms (e.g., C₂-C₁₀) and one or more triple bonds. Examples of alkynyl include, but are not limited to, ethynyl, ethynylene, 1-propynyl, 1- and 2-butynyl, and 1-methyl-2-butynyl. The term “alkoxy” refers to an —O-alkyl radical. Examples of alkoxy include, but are not limited to, methoxy, ethoxy, n-propoxy, isopropoxy, n-butoxy, iso-butoxy, sec-butoxy, and tert-butoxy. The term “acyloxy” refers to an —O—C(O)—R radical in which R can be H, alkyl, alkenyl, alkynyl, cycloalkyl, cycloalkenyl, heterocycloalkyl, heterocycloalkenyl, aryl, or heteroaryl. The term “amino” refers to NH₂, alkylamino, or arylamino. The term “alkylamino” refers to an —N(R)-alkyl radical in which R can be H, alkyl, alkenyl, alkynyl, cycloalkyl, cycloalkenyl, heterocycloalkyl, heterocycloalkenyl, aryl, or heteroaryl. The terms “amido” and “carbamido” refer to —NRC(O)R′ and —C(O)NRR′ radicals respectively, in which each of R and R′, independently, can be H, alkyl, alkenyl, alkynyl, cycloalkyl, cycloalkenyl, heterocycloalkyl, heterocycloalkenyl, aryl, or heteroaryl.

The term “cycloalkyl” refers to a monovalent or bivalent saturated hydrocarbon ring system having 3 to 30 carbon atoms (e.g., C₃-C₁₂). Examples of cycloalkyl include, but are not limited to, cyclopropyl, cyclobutyl, cyclopentyl, cyclohexyl, 1,4-cyclohexylene, cycloheptyl, cyclooctyl, and adamantine. The term “cycloalkenyl” refers to a monovalent or bivalent non-aromatic hydrocarbon ring system having 3 to 30 carbons (e.g., C₃-C₁₂) and one or more double bonds. Examples include cyclopentenyl, cyclohexenyl, and cycloheptenyl. The term “heterocycloalkyl” refers to a monovalent or bivalent nonaromatic 5-8 membered monocyclic, 8-12 membered bicyclic, or 11-14 membered tricyclic ring system having one or more heteroatoms (such as O, N, S, or Se). Examples of heterocycloalkyl groups include, but are not limited to, piperazinyl, pyrrolidinyl, dioxanyl, morpholinyl, and tetrahydrofuranyl. The term “heterocycloalkenyl” refers to a monovalent or bivalent nonaromatic 5-8 membered monocyclic, 8-12 membered bicyclic, or 11-14 membered tricyclic ring system having one or more heteroatoms (such as O, N, S, or Se) and one or more double bonds.

The term “aryl” refers to a monovalent 6-carbon monocyclic, 10-carbon bicyclic, 14-carbon tricyclic aromatic ring system. Examples of aryl groups include, but are not limited to, phenyl, naphthyl, and anthracenyl. The term “arylene” refers to a bivalent 6-carbon monocyclic, 10-carbon bicyclic, 14-carbon tricyclic aromatic ring system. The term “aryloxyl” refers to an —O-aryl. The term “arylamino” refers to an —N(R)-aryl in which R can be H, alkyl, alkenyl, alkynyl, cycloalkyl, cycloalkenyl, heterocycloalkyl, heterocycloalkenyl, aryl, or heteroaryl. The term “heteroaryl” refers to a monvalent aromatic 5-8 membered monocyclic, 8-12 membered bicyclic, or 11-14 membered tricyclic ring system having one or more heteroatoms (such as O, N, S, or Se). Examples of heteroaryl groups include pyridyl, furyl, imidazolyl, benzimidazolyl, pyrimidinyl, thienyl, quinolinyl, indolyl, and thiazolyl. The term “heteroarylene” refers to a bivalent aromatic 5-8 membered monocyclic, 8-12 membered bicyclic, or 11-14 membered tricyclic ring system having one or more heteroatoms (such as O, N, S, or Se).

Alkyl, alkenyl, alkynyl, cycloalkyl, heterocycloalkyl, cycloalkenyl, heterocycloalkenyl, amino, aryl, heteroaryl, alkylene, arylene, and heteroarylene mentioned above include both substituted and unsubstituted moieties. Possible substituents on amino, cycloalkyl, heterocycloalkyl, cycloalkenyl, heterocycloalkenyl, aryl, arylene, heteroaryl, and heteroarylene include, but are not limited to, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, C₁-C₁₀ alkoxy, aryl, aryloxy, heteroaryl, heteroaryloxy, amino, C₁-C₁₀ alkylamino, arylamino, hydroxy, halo, oxo (O═), thioxo (S═), thio, silyl, C₁-C₁₀ alkylthio, arylthio, C₁-C₁₀ alkylsulfonyl, arylsulfonyl, acylamino, aminoacyl, aminothioacyl, amidino, mercapto, amido, thioureido, thiocyanato, sulfonamido, guanidine, ureido, cyano, nitro, acyl, thioacyl, acyloxy, carbamido, carbamyl (—C(O)NH₂), carboxyl (—COOH), and carboxylic ester. On the other hand, possible substituents on alkyl, alkenyl, alkynyl, or alkylene include all of the above-recited substituents except C₁-C₁₀ alkyl. Cycloalkyl, cycloalkenyl, heterocycloalkyl, heterocycloalkenyl, aryl, and heteroaryl can also be fused with each other.

In another aspect, this invention relates to a pharmaceutical composition for use in treating a disorder such as a viral infection or cancer. The composition includes a pharmaceutically acceptable carrier and a coumarin compound of formula (I):

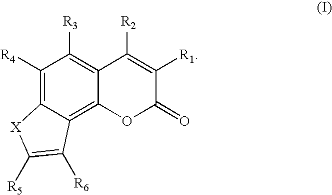

In formula (I), each of R₁, R₂, R₃, and R₄, independently, is H, alkyl, alkenyl, alkynyl, cycloalkyl, cycloalkenyl, heterocycloalkyl, heterocycloalkenyl, aryl, heteroaryl, halo, nitro, cyano, amino, hydroxy, alkoxy, aryloxy, C(O)Rₐ, C(O)ORₐ, C(O)NRₐR_{b}, C(S)R_{b}, or C(NR_{b})Rₐ, in which each of Rₐ and R_{b}, independently, is H, alkyl, alkenyl, alkynyl, hydroxy, alkoxy, amino, cycloalkyl, cycloalkenyl, heterocycloalkyl, heterocycloalkenyl, aryl, or heteroaryl; or R₁ and R₂, together with the carbon atoms to which they are bonded, are cycloalkenyl, heterocycloalkenyl, aryl, or heteroaryl; or R₂ and R₃, together with the carbon atoms to which they are bonded, are cycloalkenyl or heterocycloalkenyl; or R₃ and R₄, together with the carbon atoms to which they are bonded, are cycloalkenyl, heterocycloalkenyl, aryl, or heteroaryl; R₅ is alkyl substituted with aryl or hydroxy, alkenyl, alkynyl, cycloalkyl, cycloalkenyl, heterocycloalkyl, heterocycloalkenyl, aryl, heteroaryl, halo, nitro, cyano, amino, hydroxy, alkoxy, aryloxy, C(O)R_{c}, C(O)OR_{c}, C(O)NR_{c}R_{d}, C(S)R_{d}, or C(NR_{d})R_{c}, in which each of R_{c} and R_{d}, independently, is H, alkyl, alkenyl, alkynyl, hydroxy, alkoxy, amino, cycloalkyl, cycloalkenyl, heterocycloalkyl, heterocycloalkenyl, aryl, or heteroaryl; R₆ is alkenyl, alkynyl, cycloalkyl, cycloalkenyl, heterocycloalkyl, heterocycloalkenyl, heteroaryl, halo, nitro, cyano, amino, hydroxy, alkoxy, aryloxy, C(O)R_{c}, C(O)OR_{c}, C(O)NR_{c}R_{d}, C(S)R_{d}, C(NR_{d})R_{c}, or aryl substituted with alkyl at the 3-position of the aryl, halo, nitro, cyano, amino, cycloalkyl, aryl, or heteroaryl; and X is O, S, or N(Rₑ), in which Rₑ is H, alkyl, cycloalkyl, heterocycloalkyl, aryl, or heteroaryl.

One subset of the just-described coumarin compounds, used in a pharmaceutical composition, includes those in which R₅ is alkyl substituted with aryl or hydroxy, cycloalkyl, aryl, halo, C(O)R_{c}, C(O)OR_{c}, C(O)NR_{c}R_{d}, C(S)R_{d}, or C(NR_{d})R_{c}. In these compounds, R₅ can be C(O)R_{c} or C(O)OR_{c}, in which R_{c} can be aryl or heteroaryl; R₆ can be cycloalkyl, heteroaryl, or aryl substituted with alkyl at the 3-position of the aryl, halo, nitro, cyano, amino, cycloalkyl, aryl, or heteroaryl; R₆ can be heteroaryl or phenyl substituted with alkyl at the 3-position of the phenyl, halo, nitro, cyano, or amino; each of R₁, R₂, R₃, and R₄, independently, can be H, alkyl, alkenyl, cycloalkyl, cycloalkenyl, heterocycloalkyl, aryl, hydroxy, alkoxy, halo, cyano, nitro, or C(O)H; R₂ can be alkyl or C(O)H; or X can be O.

Two other subsets of these coumarin compounds include those in which X is O and those in which each of R₁, R₂, R₃, and R₄, independently, is H, alkyl, alkenyl, cycloalkyl, cycloalkenyl, heterocycloalkyl, aryl, hydroxy, alkoxy, halo, cyano, nitro, or C(O)H.

In still another aspect, this invention relates to a pharmaceutical composition including a pharmaceutically acceptable carrier and a coumarin compound of formula (I):

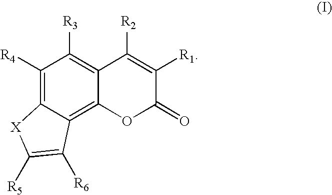

In this formula, each of R₁, R₂, R₃, and R₄, independently, is H, alkyl, alkenyl, alkynyl, cycloalkyl, cycloalkenyl, heterocycloalkyl, heterocycloalkenyl, aryl, heteroaryl, halo, nitro, cyano, amino, hydroxy, alkoxy, aryloxy, C(O)Rₐ, C(O)ORₐ, C(O)NRₐR_{b}, C(S)R_{b}, or C(NR_{b})Rₐ, in which each of Rₐ and R_{b}, independently, is H, alkyl, alkenyl, alkynyl, hydroxy, alkoxy, amino, cycloalkyl, cycloalkenyl, heterocycloalkyl, heterocycloalkenyl, aryl, or heteroaryl; or R₁ and R₂, together with the carbon atoms to which they are bonded, are cycloalkenyl, heterocycloalkenyl, aryl, or heteroaryl; or R₂ and R₃, together with the carbon atoms to which they are bonded, are cycloalkenyl or heterocycloalkenyl; or R₃ and R₄, together with the carbon atoms to which they are bonded, are cycloalkenyl, heterocycloalkenyl, aryl, or heteroaryl; R₅ is alkyl substituted with aryl or hydroxy, alkenyl, alkynyl, cycloalkyl, cycloalkenyl, heterocycloalkyl, heterocycloalkenyl, aryl, heteroaryl, halo, nitro, cyano, amino, hydroxy, alkoxy, aryloxy, C(O)R_{c}, C(O)OR_{c}, C(O)NR_{d}Rₑ, C(S)R_{d}, or C(NRₑ)R_{d}, in which R_{c} is cycloalkyl, cycloalkenyl, heterocycloalkyl, heterocycloalkenyl, heteroaryl, or aryl substituted with alkyl, halo, nitro, cyano, amino, amido, cycloalkyl, aryl, heteroaryl, hydroxy, alkoxy, acyloxy, silyloxy, or phosphate at the 2- or 3-position of the aryl, and each of R_{d} and Rₑ, independently, is H, alkyl, alkenyl, alkynyl, hydroxy, alkoxy, amino, cycloalkyl, cycloalkenyl, heterocycloalkyl, heterocycloalkenyl, aryl, or heteroaryl; R₆ is H, alkyl, alkenyl, alkynyl, cycloalkyl, cycloalkenyl, heterocycloalkyl, heterocycloalkenyl, aryl, heteroaryl, halo, nitro, cyano, amino, hydroxy, alkoxy, or aryloxy; and X is O, S, or N(R_{f}), in which R_{f} is H, alkyl, cycloalkyl, heterocycloalkyl, aryl, or heteroaryl.

One subset of the just-described coumarin compounds includes those in which R₆ is heteroaryl or aryl. In these compounds, R₅ can be C(O)R_{c} or C(O)OR_{c}, in which R_{c} can be heteroaryl or aryl substituted with halo, nitro, cyano, amino, amido, cycloalkyl, aryl, heteroaryl, hydroxy, alkoxy, acyloxy, silyloxy, or phosphate at the 2- or 3-position of the aryl; R₅ can be alkyl substituted with aryl, C(S)R_{d}, or C(NRₑ)R_{d}; each of R₁, R₂, R₃, and R₄, independently, can be H, alkyl, alkenyl, cycloalkyl, cycloalkenyl, heterocycloalkyl, aryl, hydroxy, alkoxy, halo, cyano, nitro, or C(O)H; R₂ can be alkyl or C(O)H; or X can be O.

Two other subsets of these coumarin compounds include those in which X is O and those in which each of R₁, R₂, R₃, and R₄, independently, is H, alkyl, alkenyl, cycloalkyl, cycloalkenyl, heterocycloalkyl, aryl, hydroxy, alkoxy, halo, cyano, nitro, or C(O)H.

Further, this invention features including a pharmaceutically acceptable carrier and a coumarin compound of formula (I):

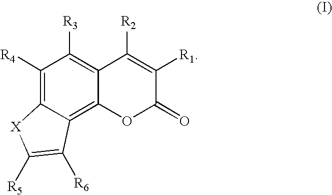

In formula (I), each of R₁, R₃, and R₄, independently, is H, alkyl, alkenyl, alkynyl, cycloalkyl, cycloalkenyl, heterocycloalkyl, heterocycloalkenyl, aryl, heteroaryl, halo, nitro, cyano, amino, hydroxy, alkoxy, aryloxy, C(O)Rₐ, C(O)ORₐ, C(O)NRₐR_{b}, C(S)R_{b}, or C(NR_{b})Rₐ, in which each of I, and R_{b}, independently, is H, alkyl, alkenyl, alkynyl, hydroxy, alkoxy, amino, cycloalkyl, cycloalkenyl, heterocycloalkyl, heterocycloalkenyl, aryl, or heteroaryl; R₂ is H, C₂-C₁₀ alkyl, alkenyl, alkynyl, cycloalkyl, cycloalkenyl, heterocycloalkyl, heterocycloalkenyl, heteroaryl, halo, nitro, cyano, amino, hydroxy, alkoxy, aryloxy, C(O)Rₐ, C(O)ORₐ, C(O)NRₐR_{b}, C(S)R_{b}, or C(NR_{b})Rₐ; or R₁ and R₂, together with the carbon atoms to which they are bonded, are cycloalkenyl, heterocycloalkenyl, aryl, or heteroaryl; or R₂ and R₃, together with the carbon atoms to which they are bonded, are cycloalkenyl or heterocycloalkenyl; or R₃ and R₄, together with the carbon atoms to which they are bonded, are cycloalkenyl, heterocycloalkenyl, aryl, or heteroaryl; R₅ is C(O)R_{c}, C(O)OR_{c}, C(O)NR_{d}Rₑ, C(S)R_{d}, or C(NRₑ)R_{d}, in which, R_{c} is aryl or heteroaryl, and each of R_{d} and Rₑ, independently, is H, alkyl, alkenyl, alkynyl, hydroxy, alkoxy, amino, cycloalkyl, cycloalkenyl, heterocycloalkyl, heterocycloalkenyl, aryl, or heteroaryl; R₆ is aryl or heteroaryl; and X is O, S, or N(R_{f}), in which R_{f} is H, alkyl, cycloalkyl, heterocycloalkyl, aryl, or heteroaryl.

One subset of the just-described coumarin compounds includes those in which X is O and R₂ is C₂-C₁₀ alkyl or C(O)H. In these compounds, each of R₁, R₃, and R₄, independently, can be H, alkyl, alkenyl, cycloalkyl, cycloalkenyl, heterocycloalkyl, aryl, hydroxy, alkoxy, halo, cyano, nitro, or C(O)H.

Another subset of these coumarin compounds includes those in which X is O and each of R₁, R₃, and R₄, independently, is H, alkyl, alkenyl, cycloalkyl, cycloalkenyl, heterocycloalkyl, aryl, hydroxy, alkoxy, halo, cyano, nitro, or C(O)H.

The coumarin compounds described above include the compounds themselves, as well as their salts, their solvates, and their prodrugs, if applicable. A salt, for example, can be formed between an anion and a positively charged group (e.g., amino) on a coumarin compound. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate. Likewise, a salt can also be formed between a cation and a negatively charged group (e.g., carboxylate) on a coumarin compound. Suitable cations include sodium ion, potassium ion, magnesium ion, calcium ion, and an ammonium cation such as tetramethylammonium ion. The coumarin compounds also include those salts containing quaternary nitrogen atoms. Examples of prodrugs include esters and other pharmaceutically acceptable derivatives, which, upon administration to a subject, are capable of providing active coumarin compounds.

Also within the scope of this invention is the therapeutic use of the above-described coumarin compounds and use of the compounds for the manufacture of a medicament for treating a disorder such as an infection with a virus.

8-Benzoyl-4-methyl-9-phenylcyclopenta[h]chromen-2(7H)-one and its analogs, as well as their therapeutic use as described above, are also contemplated.

The details of one or more embodiments of the invention are set forth in the description below. Other features, objects, and advantages of the invention will be apparent from the description and the claims.

### DETAILED DESCRIPTION

Shown below are exemplary compounds of this invention:

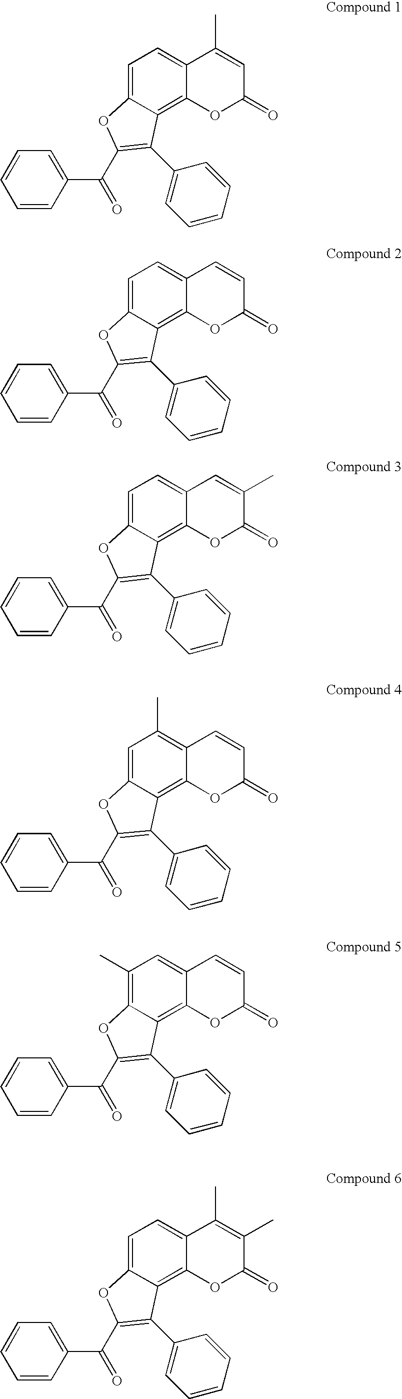

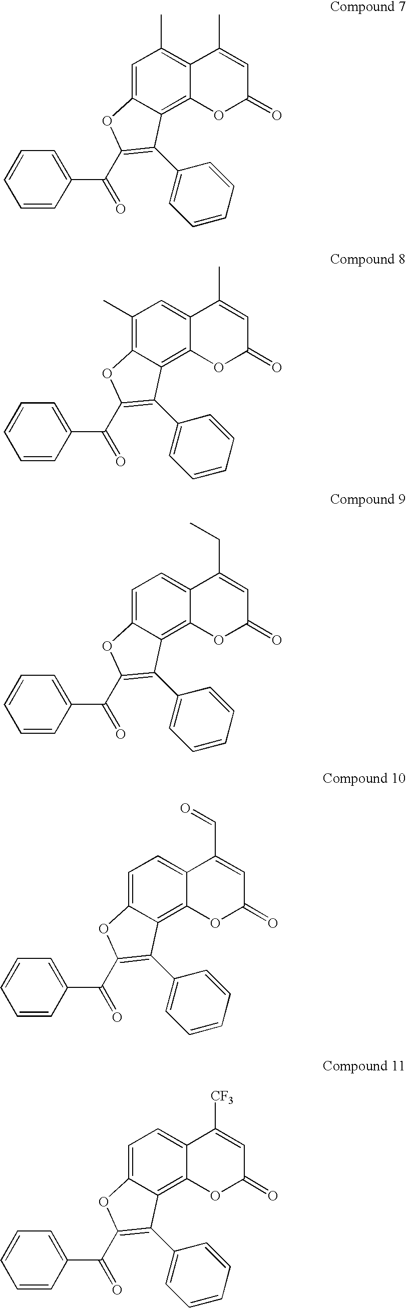

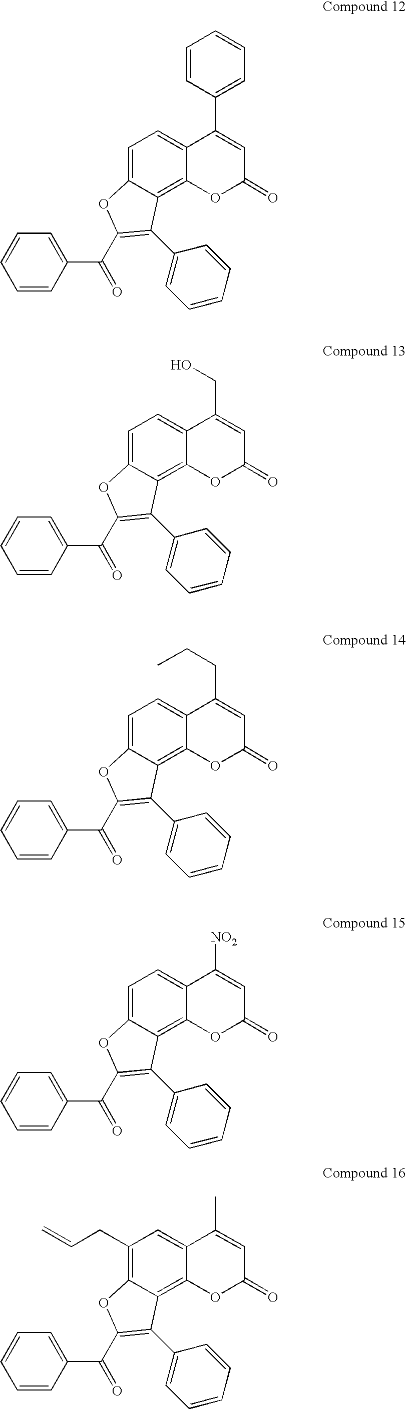

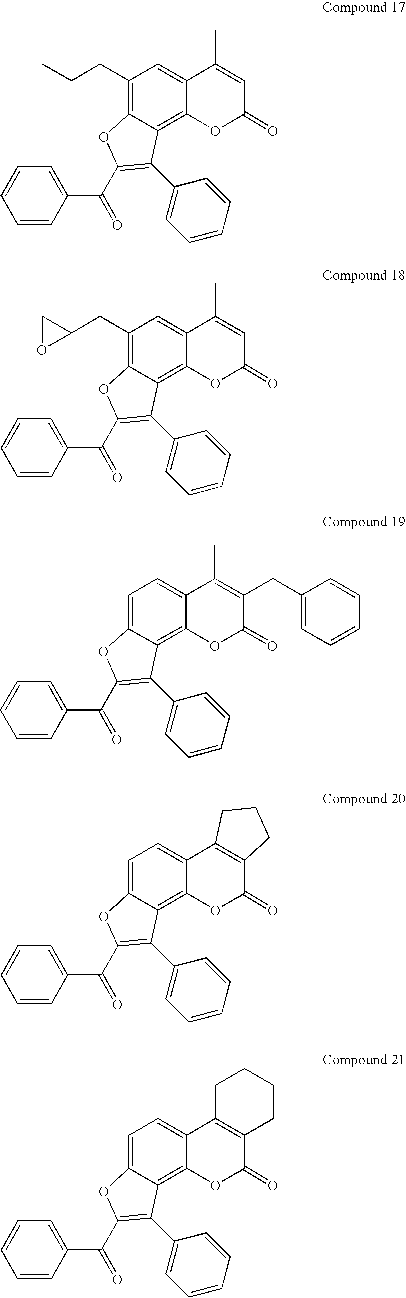

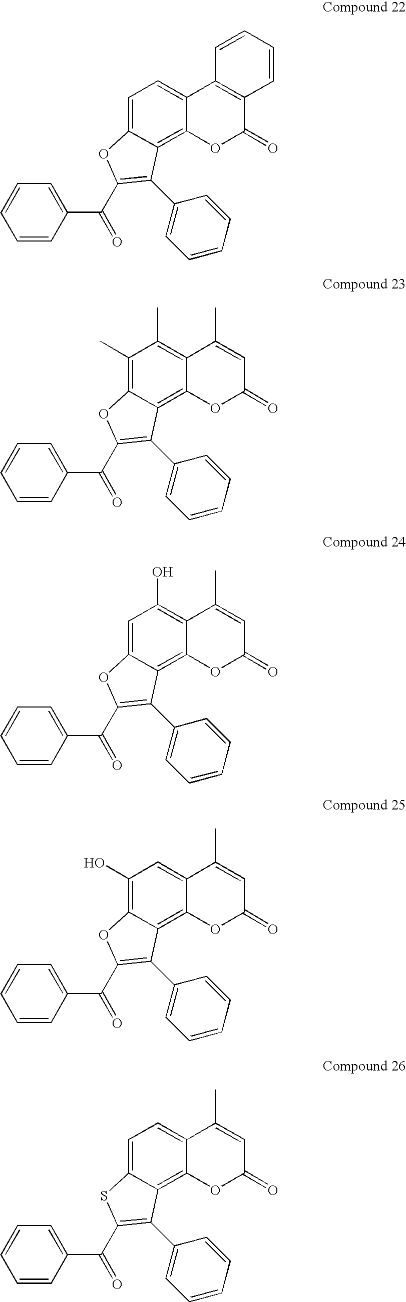

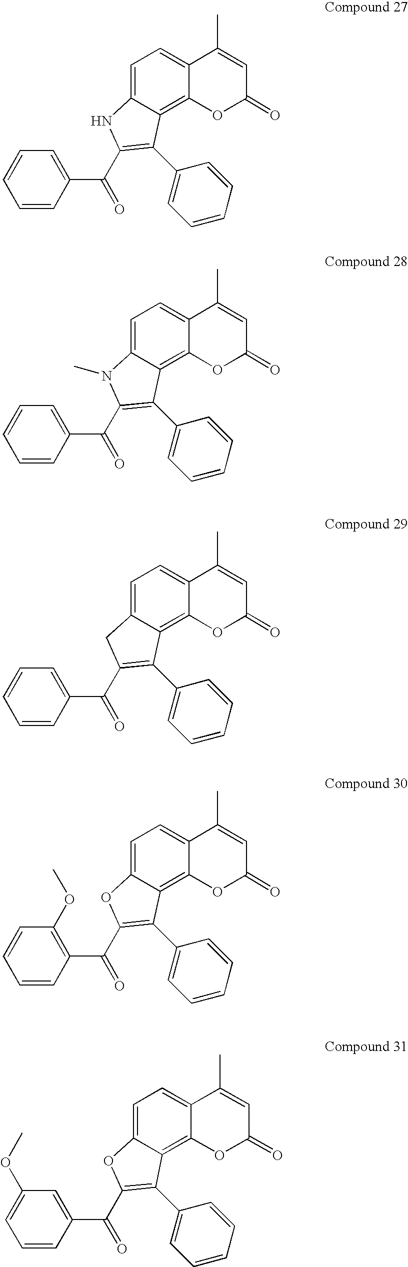

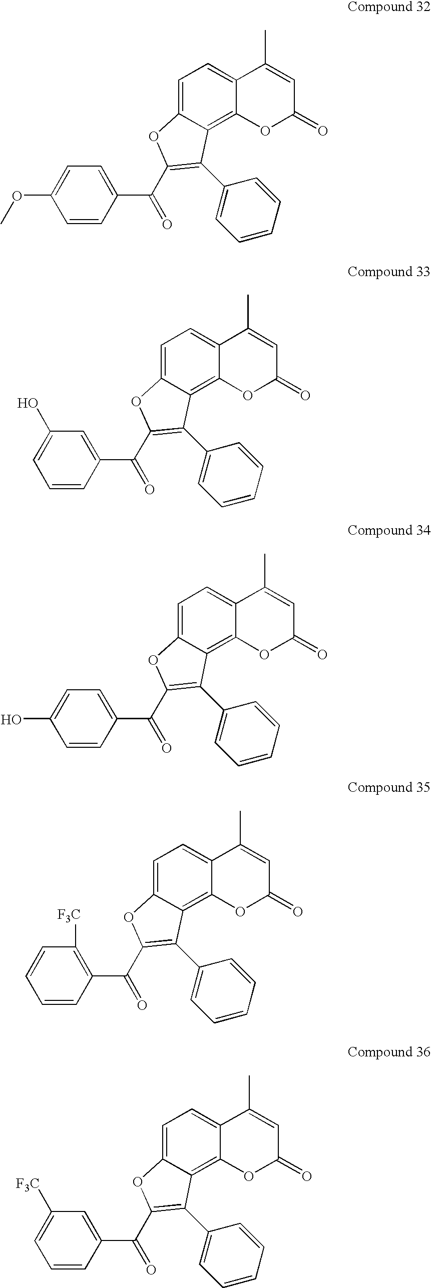

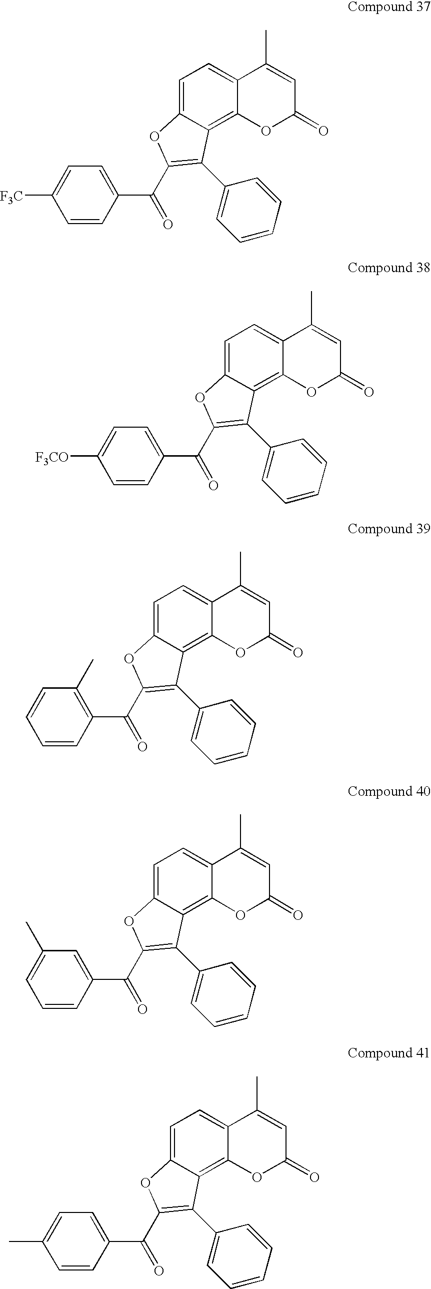

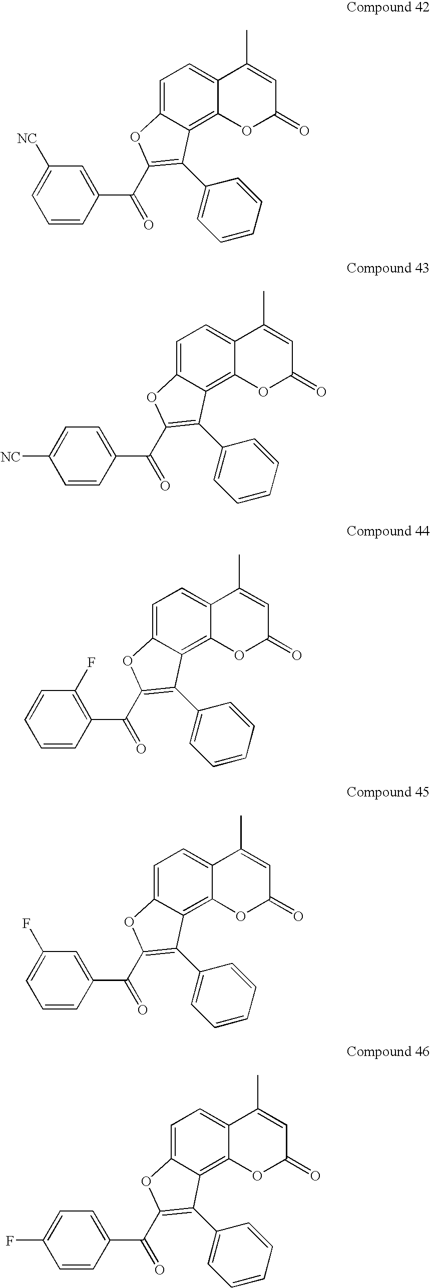

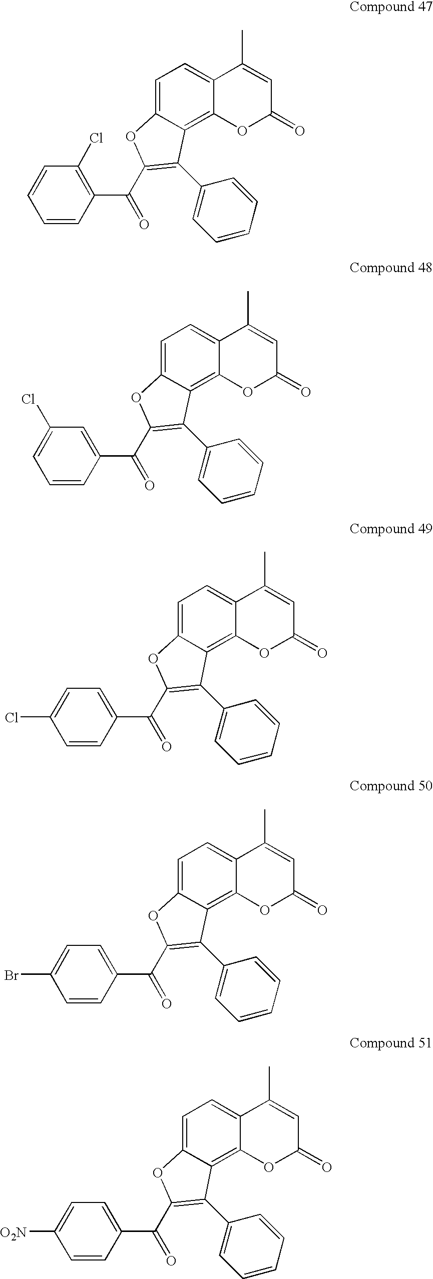

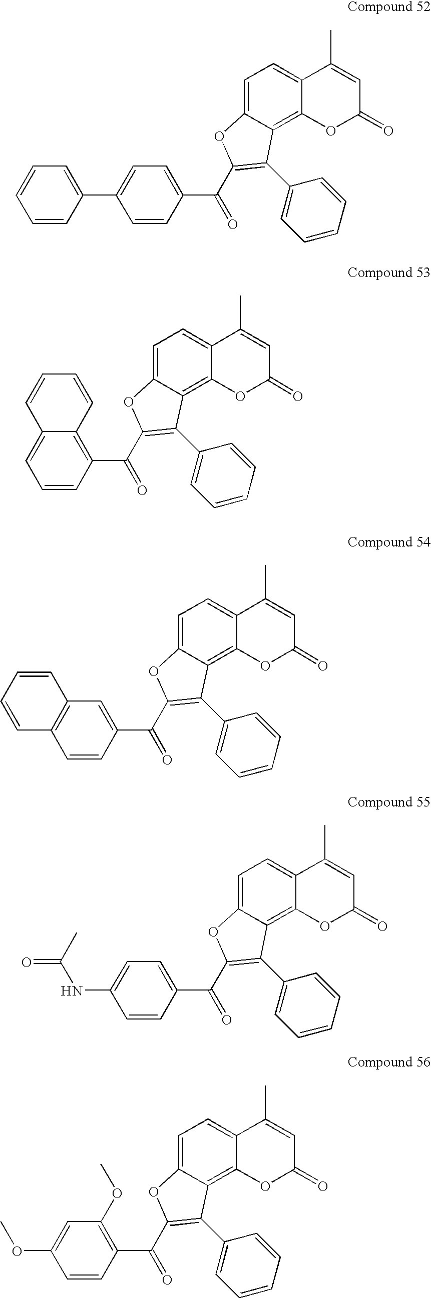

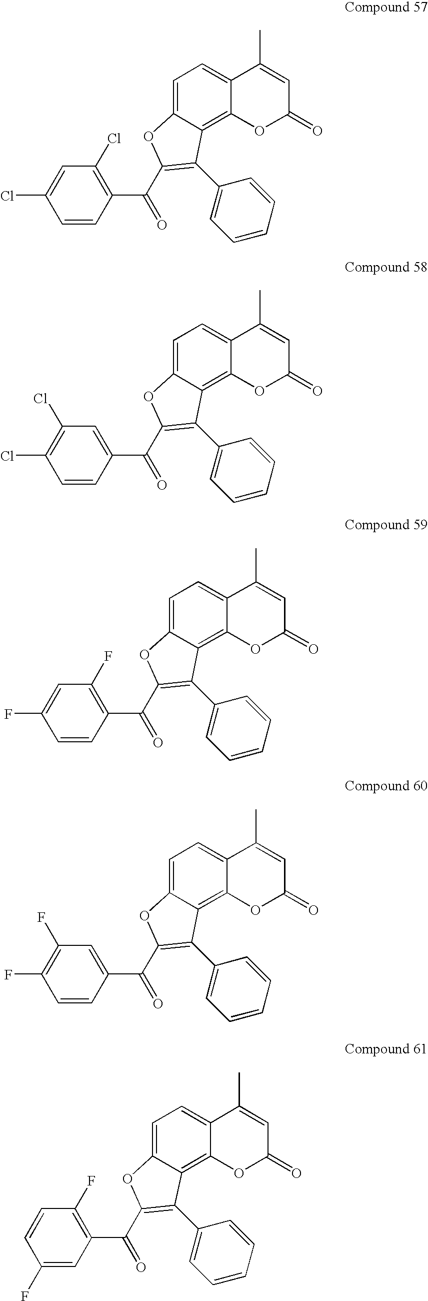

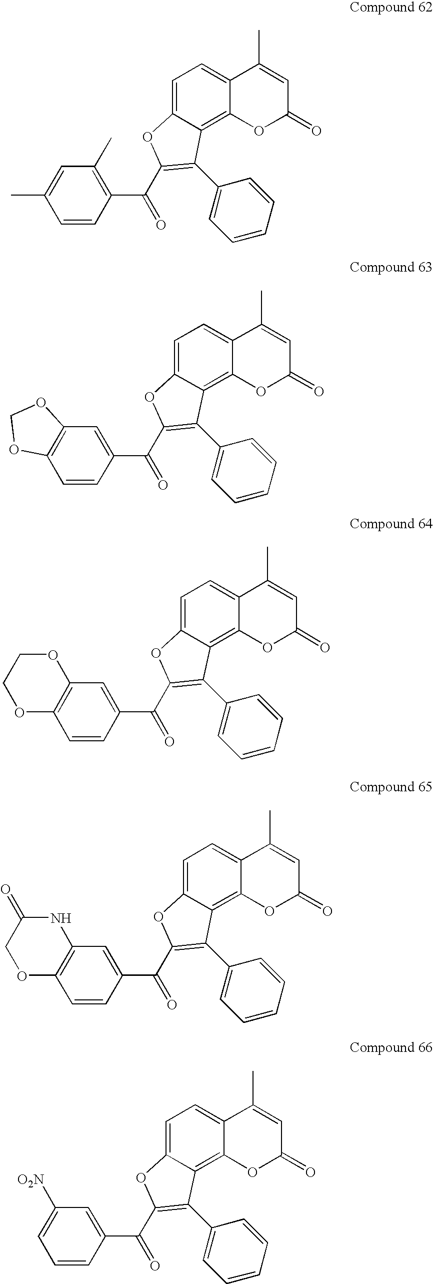

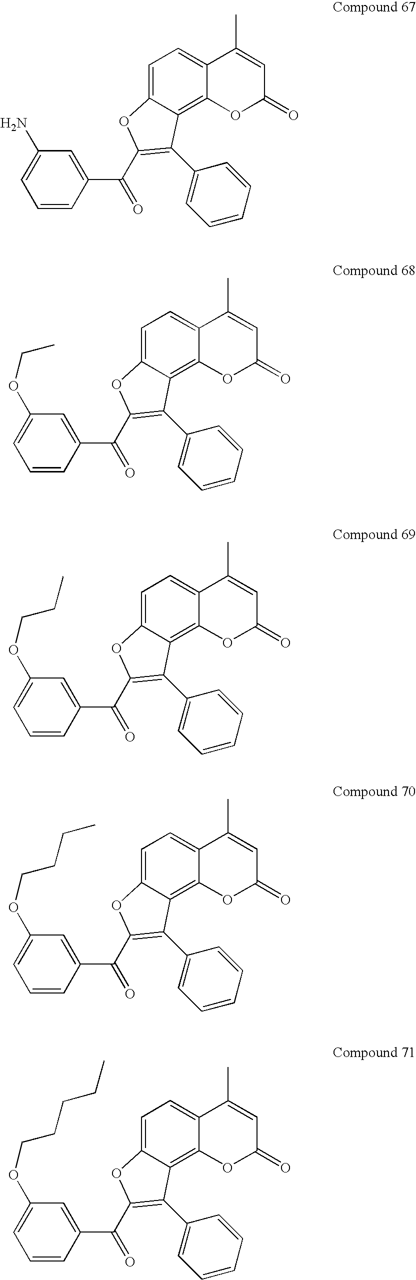

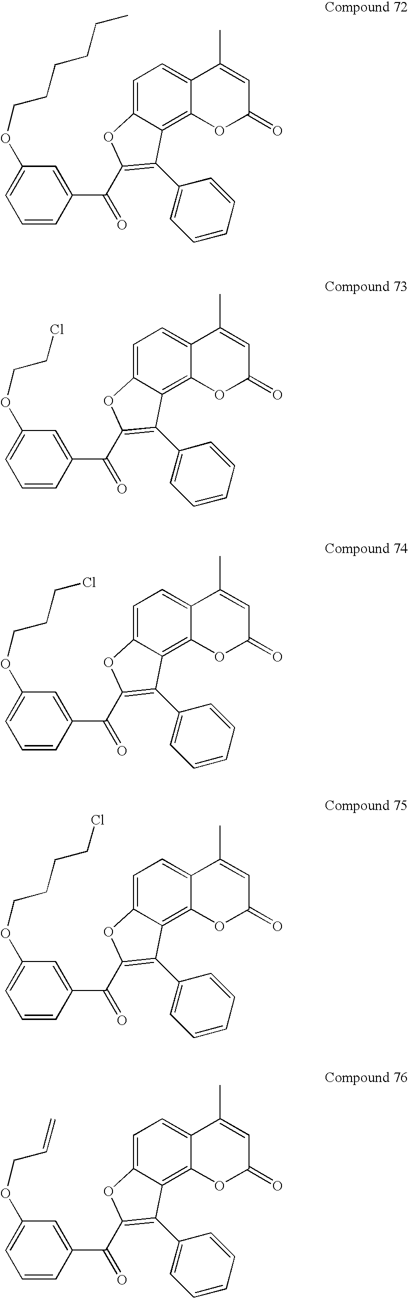

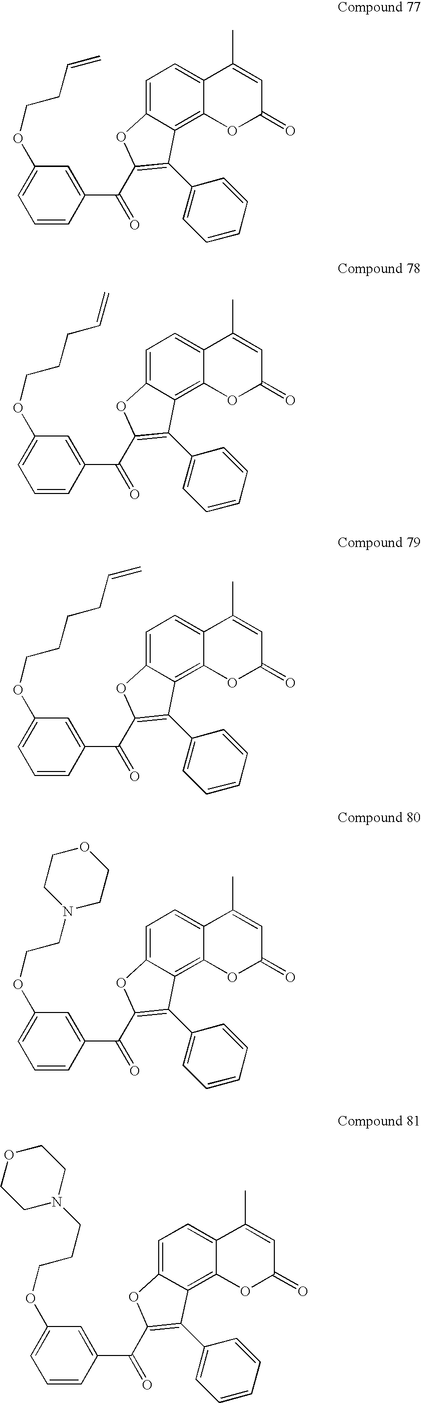

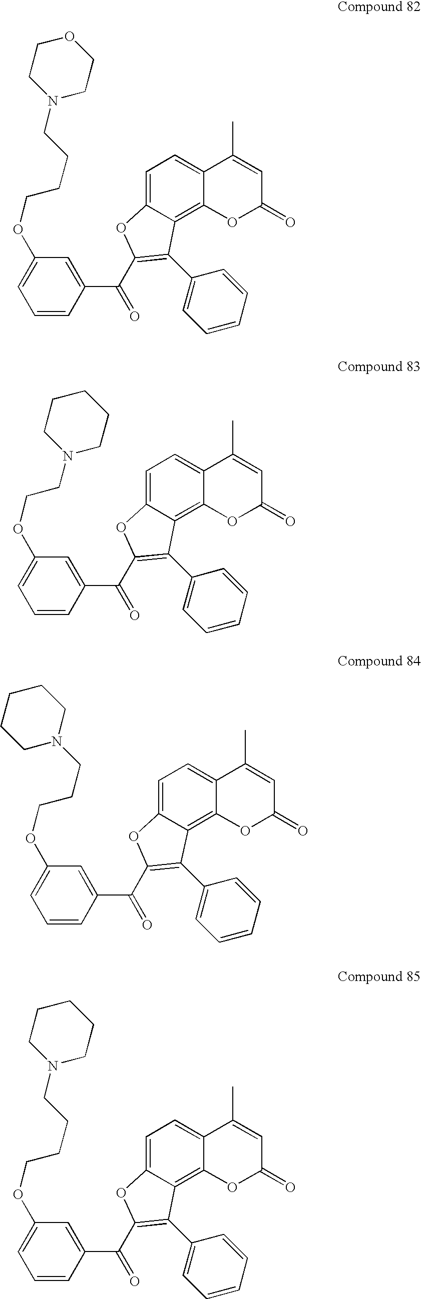

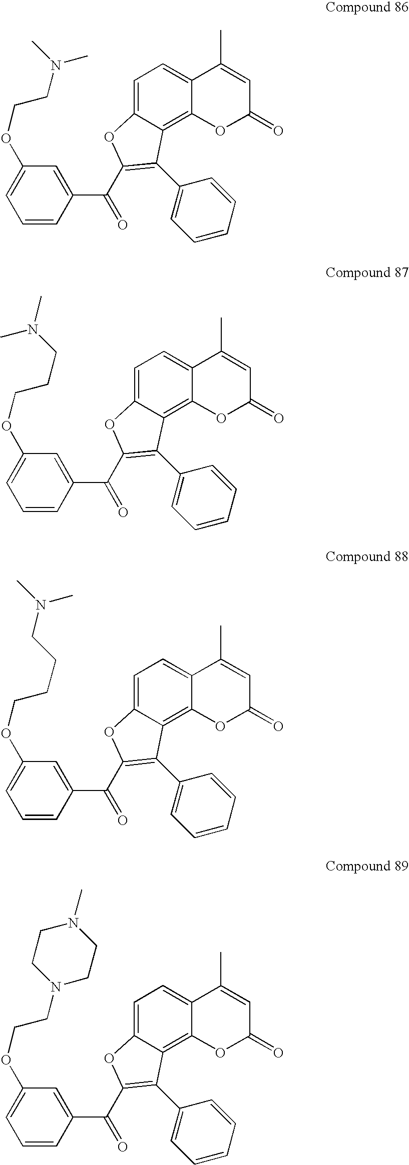

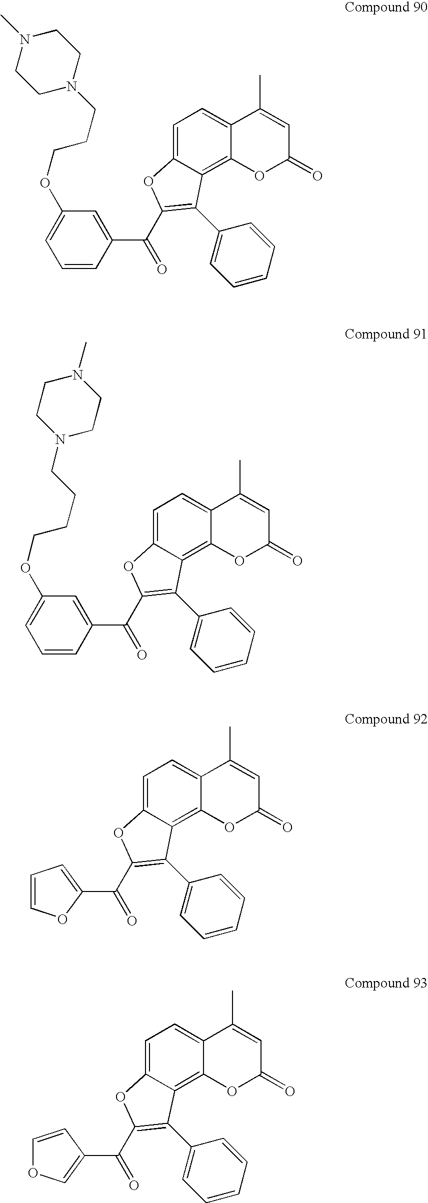

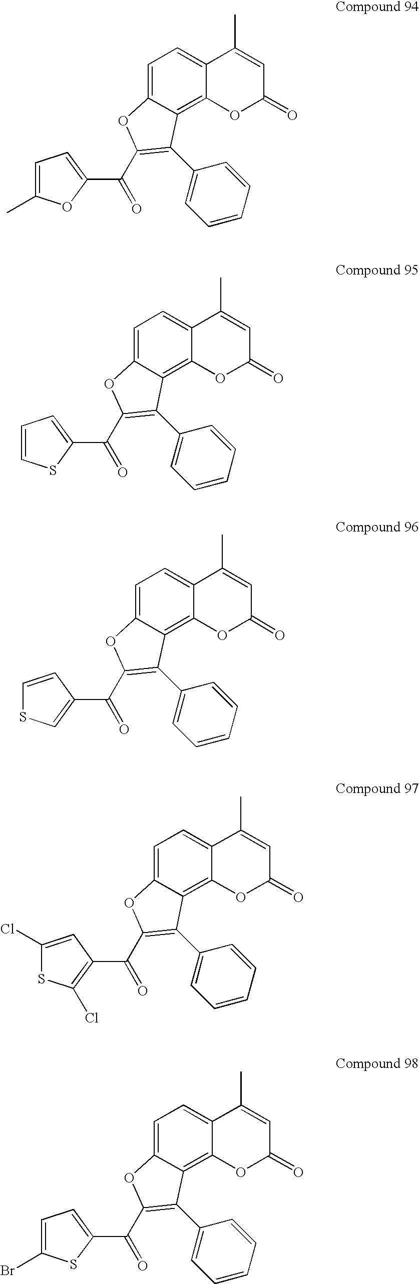

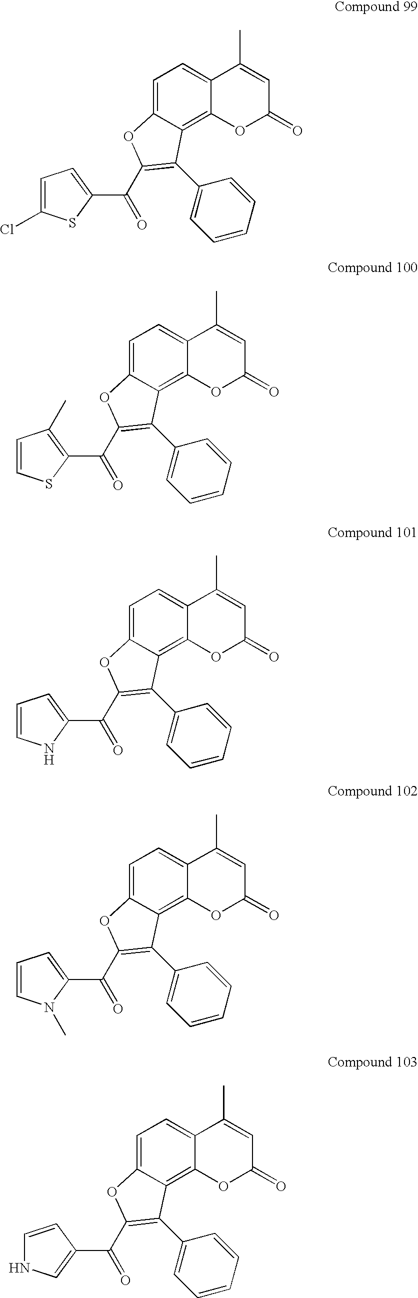

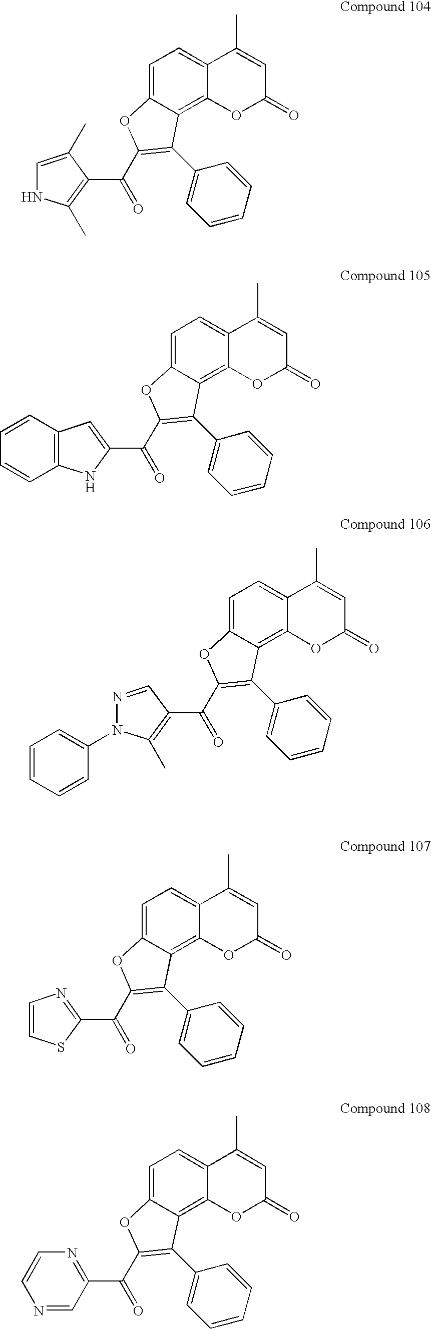

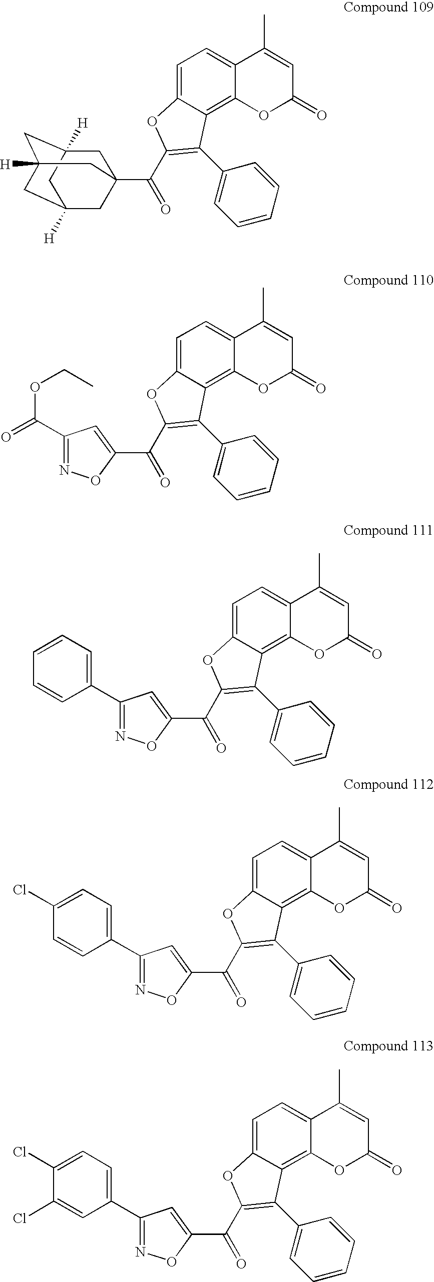

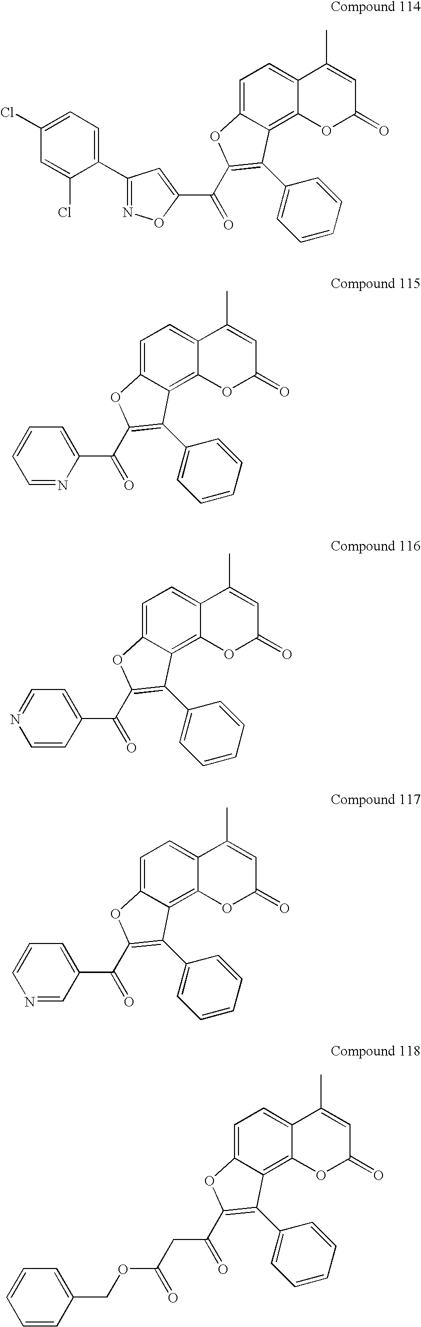

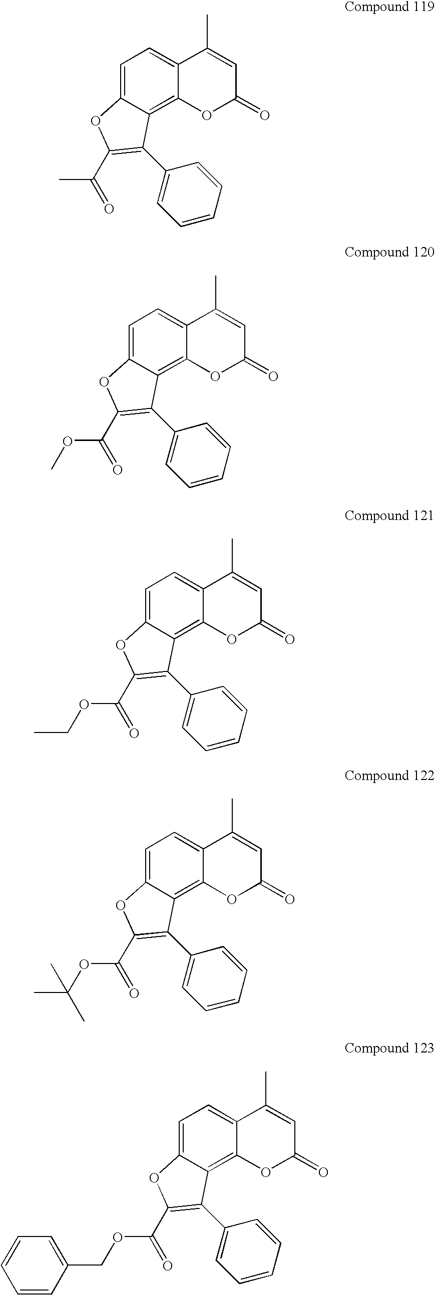

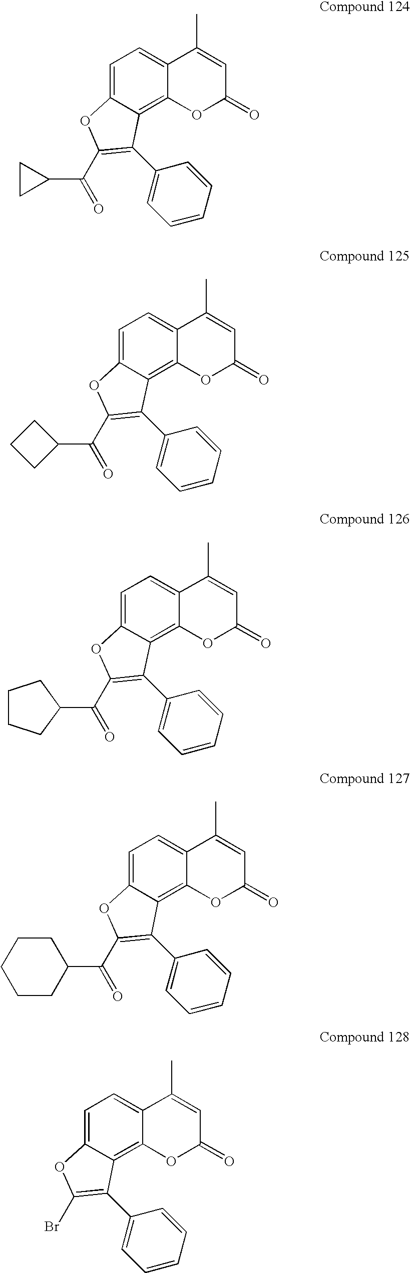

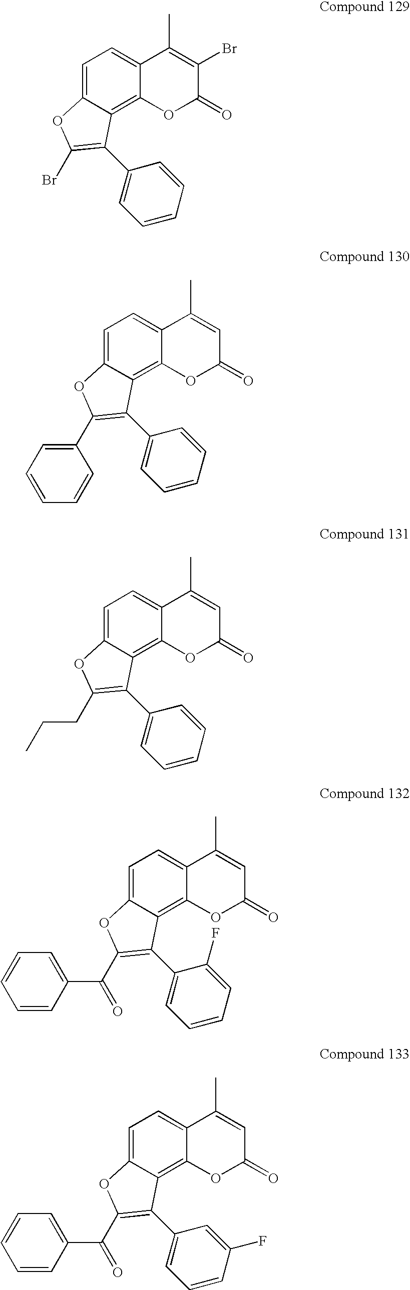

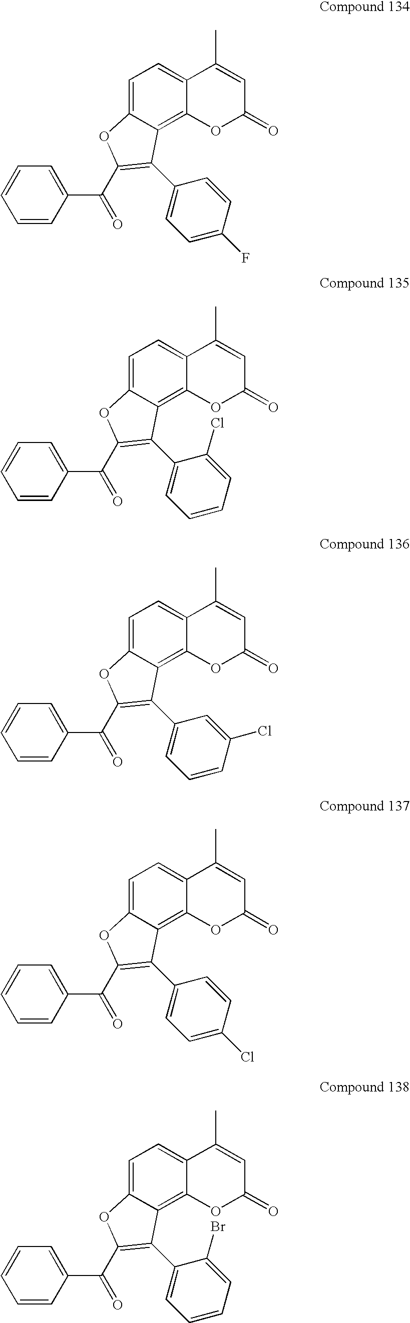

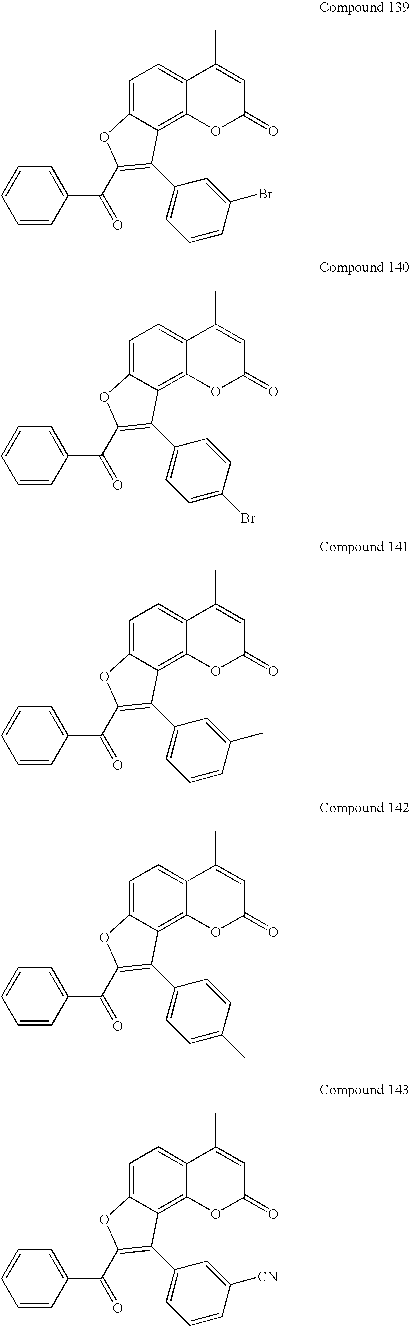

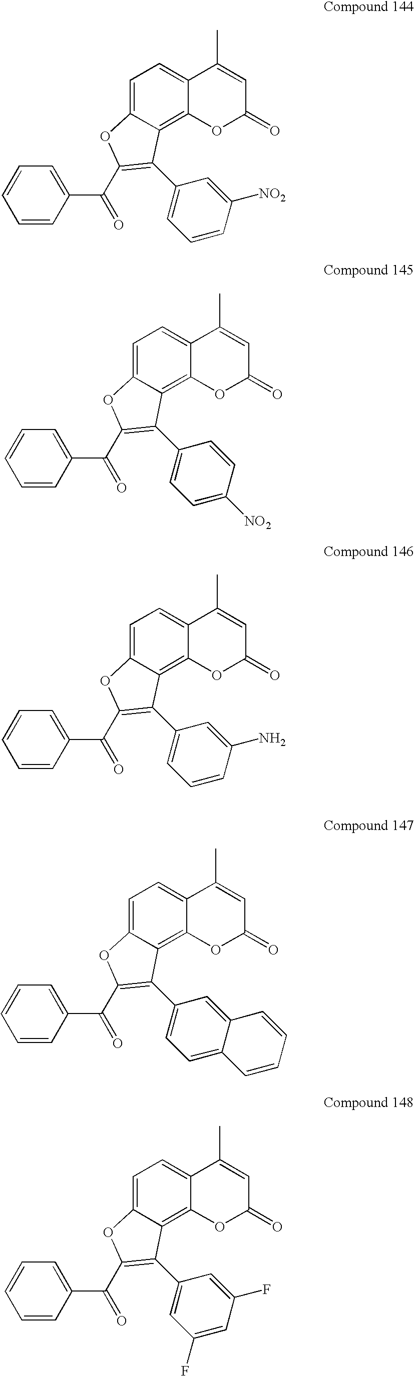

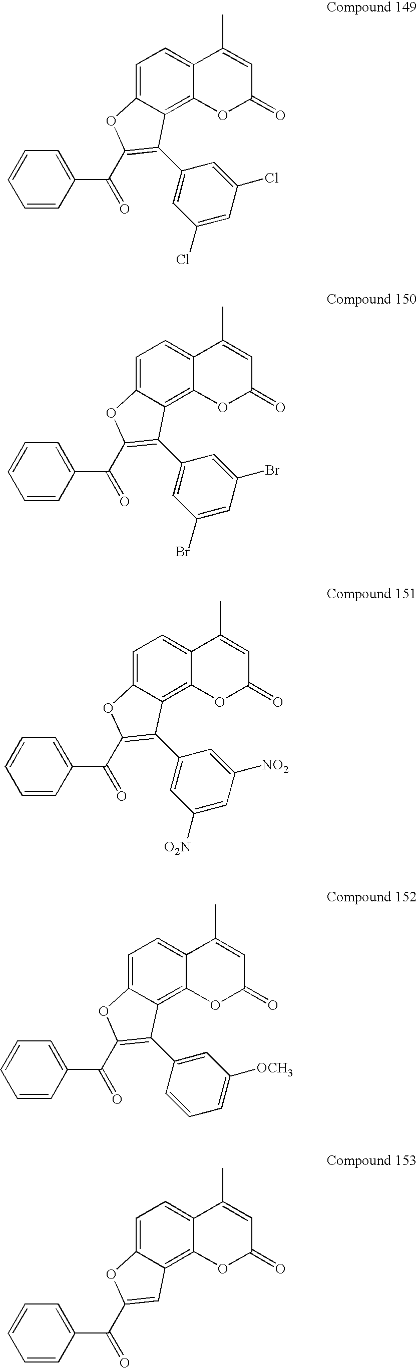

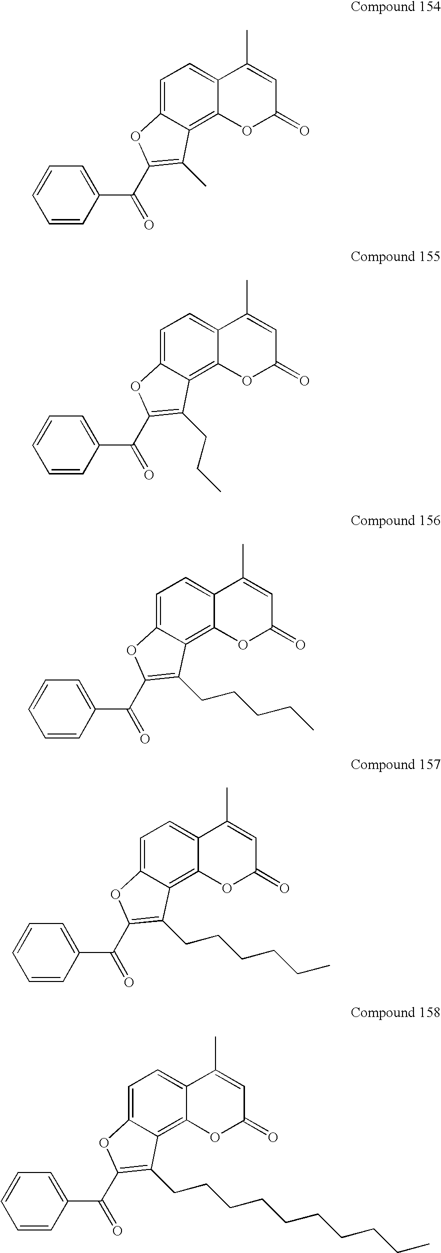

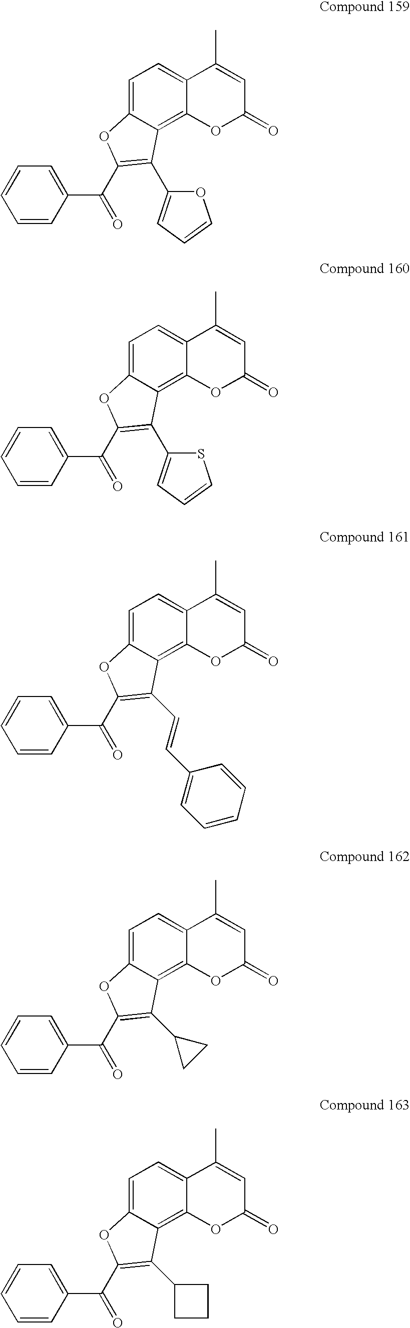

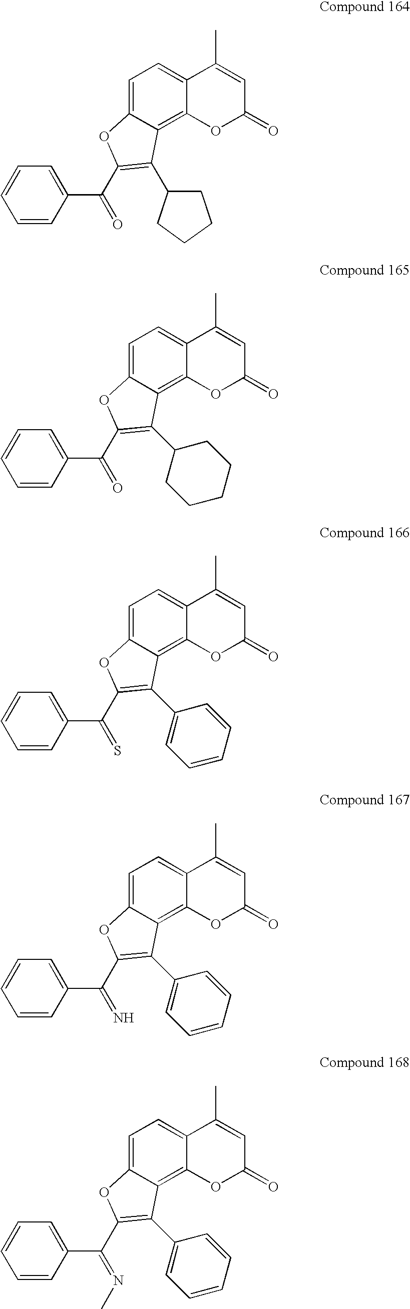

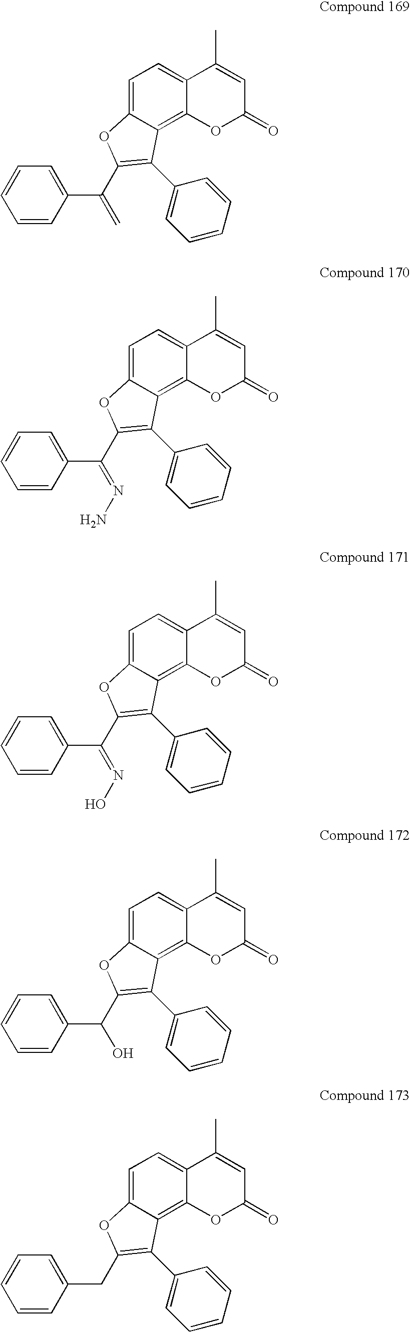

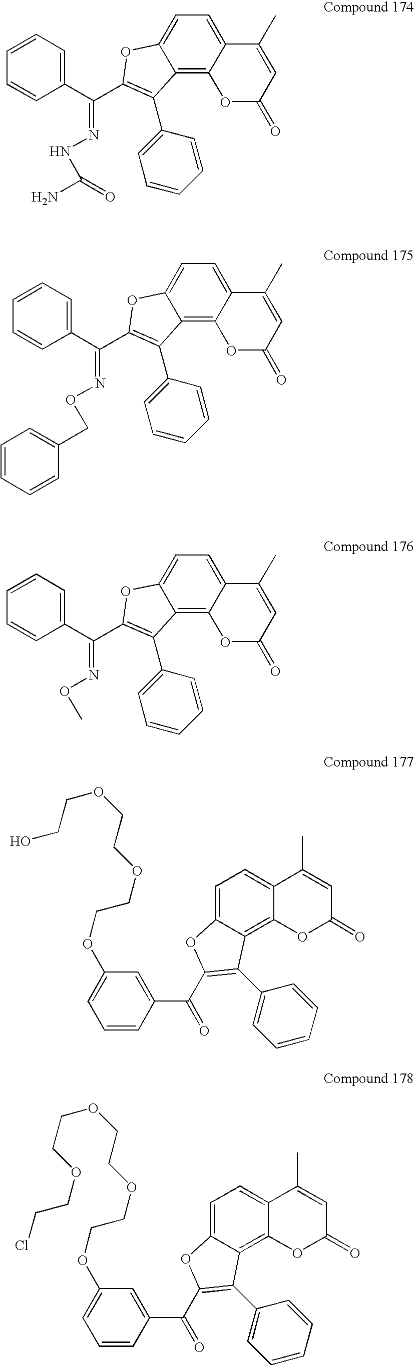

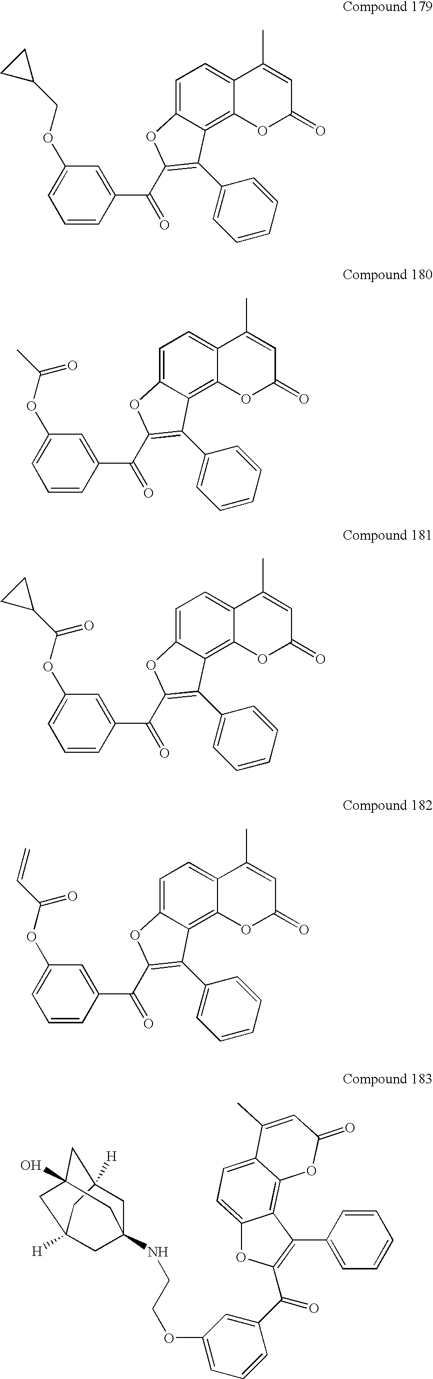

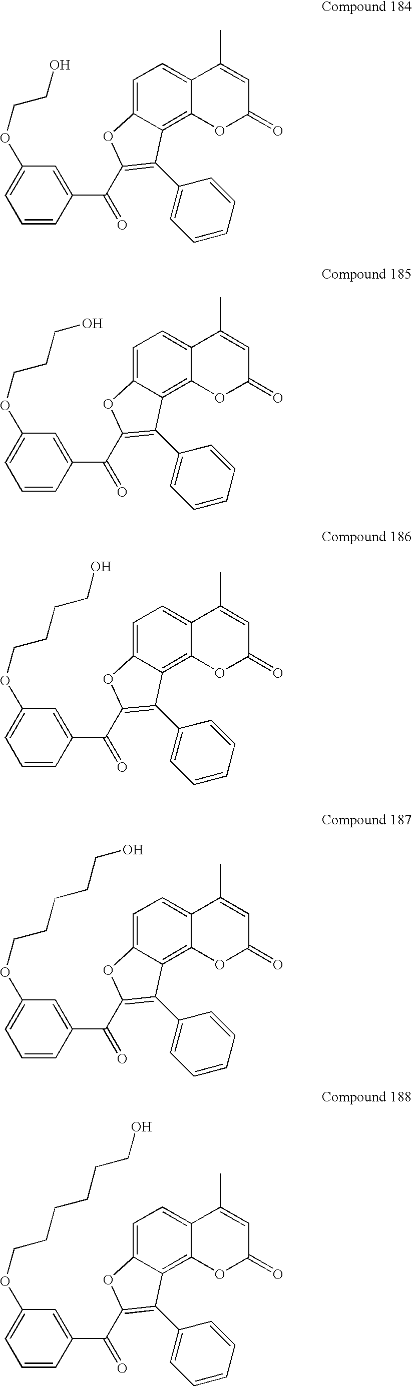

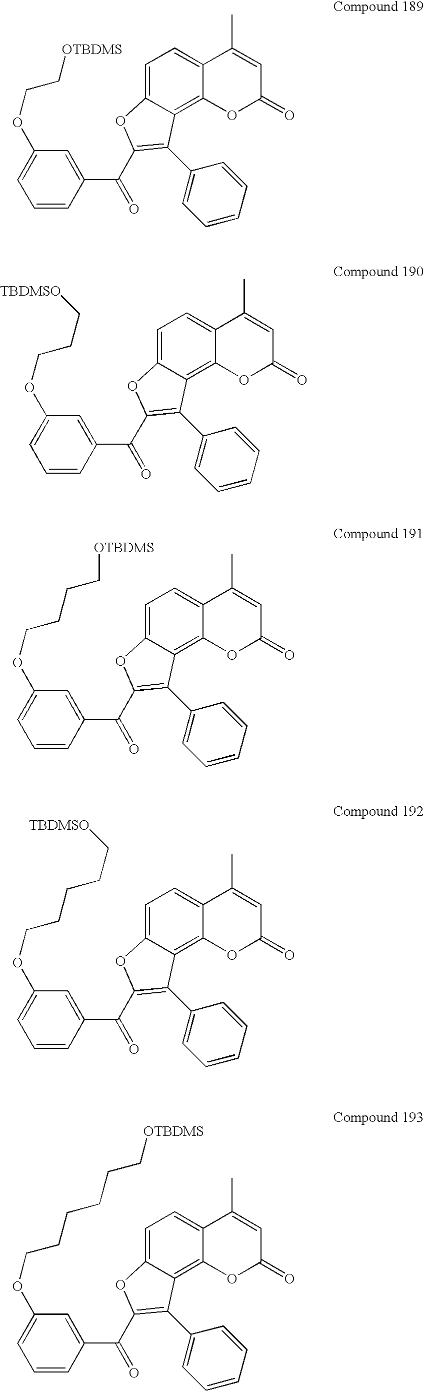

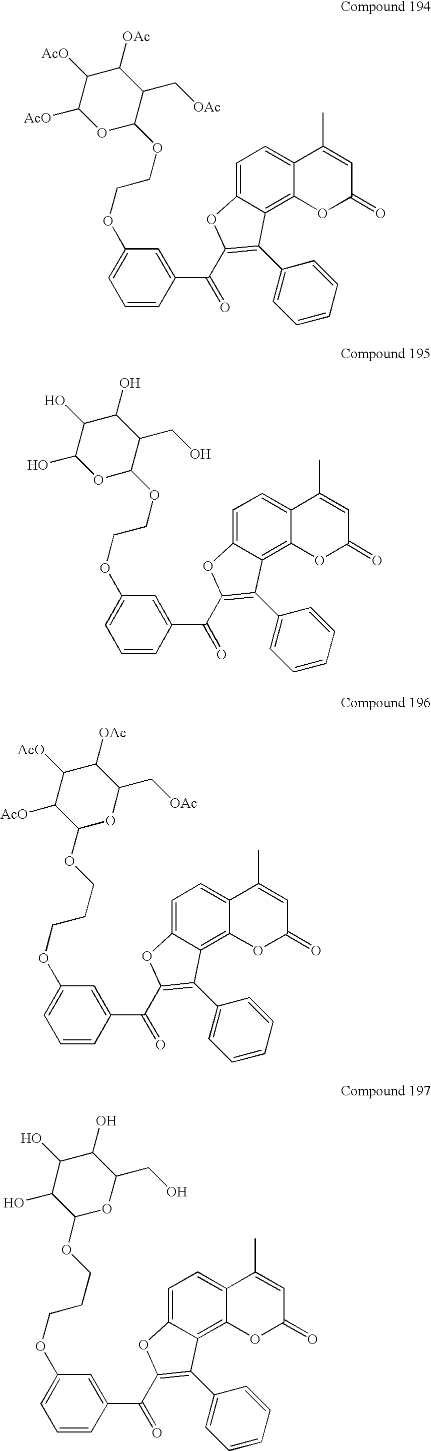

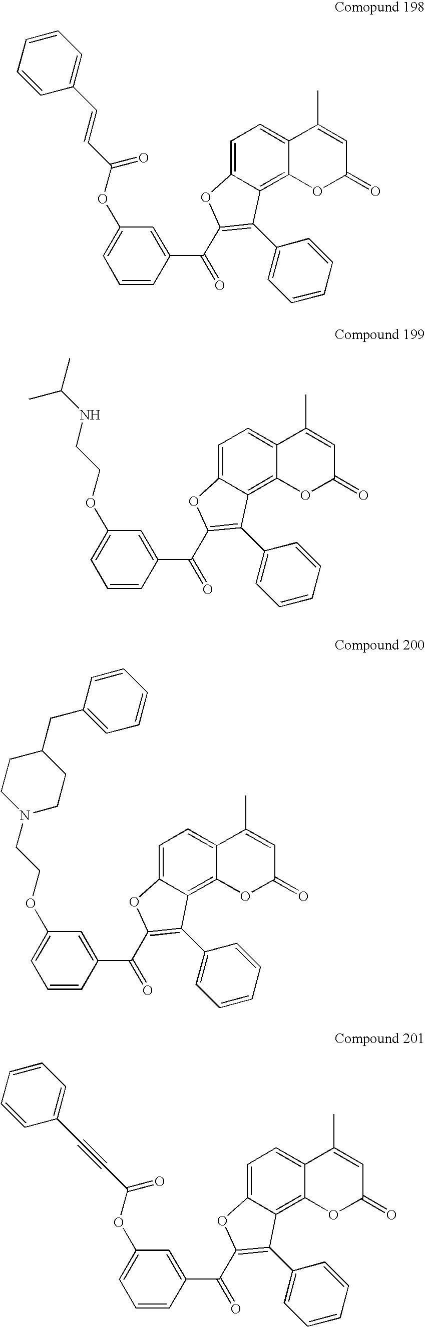

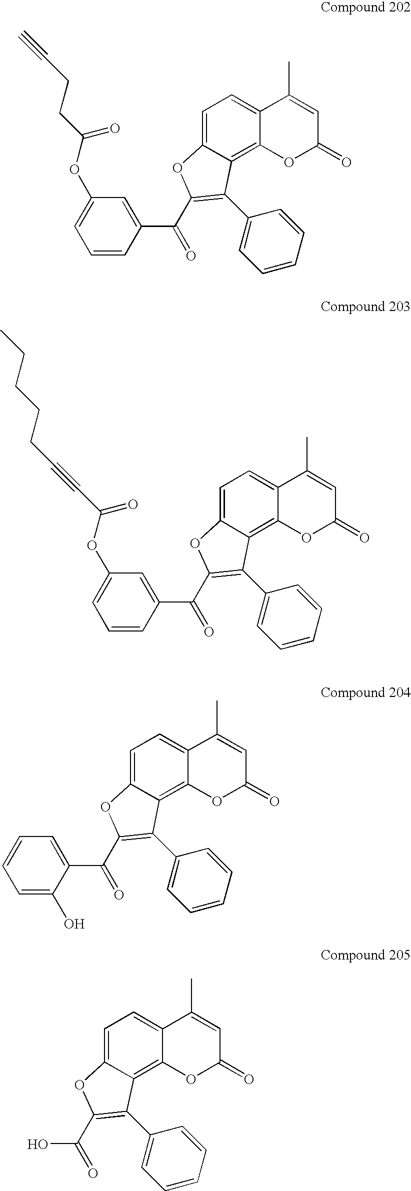

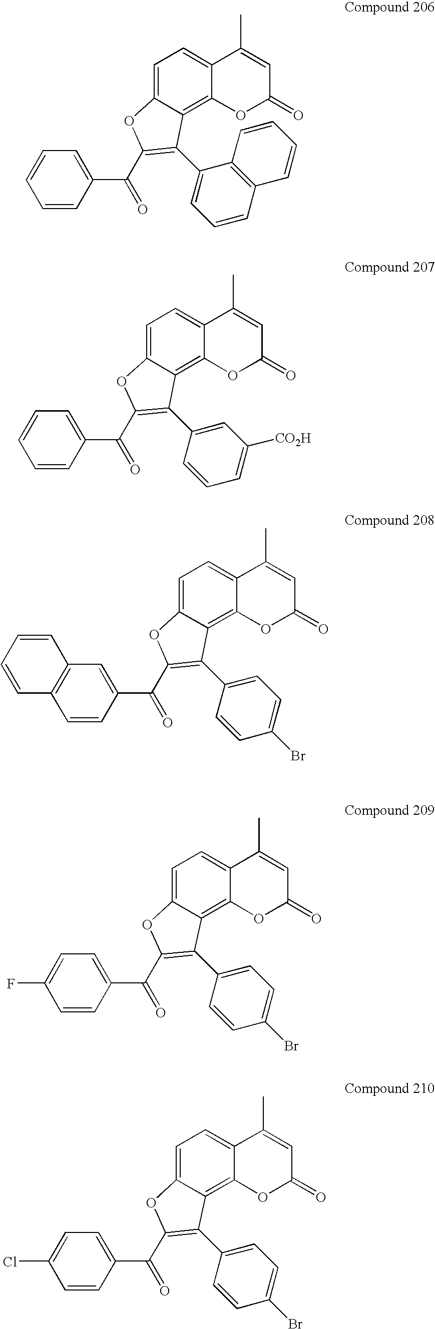

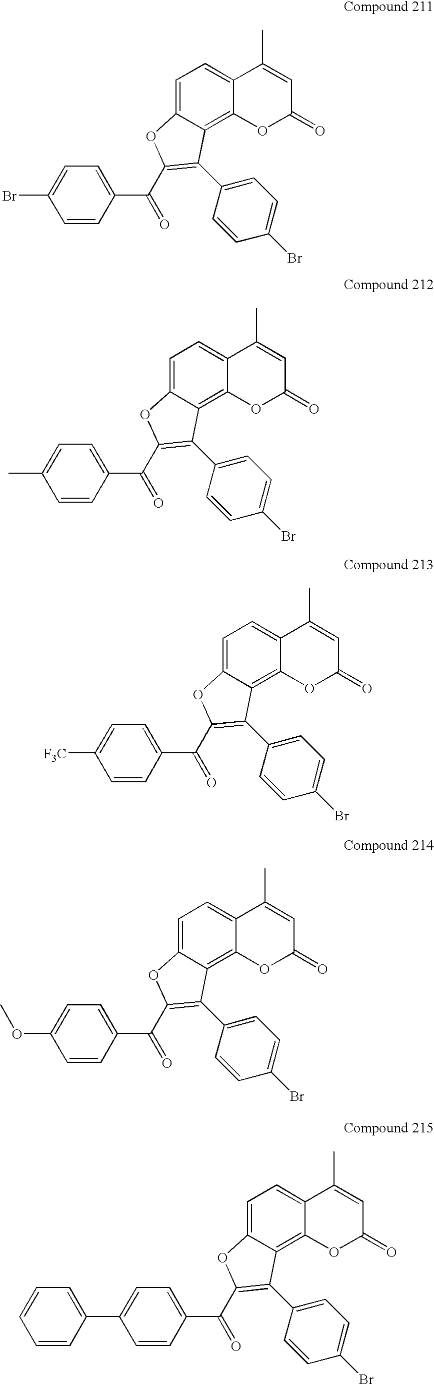

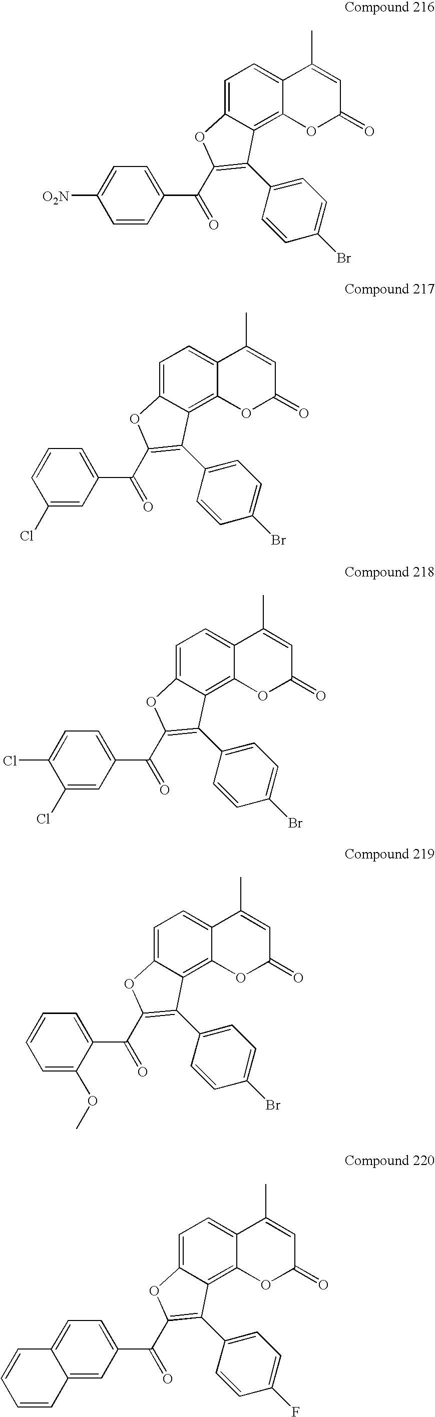

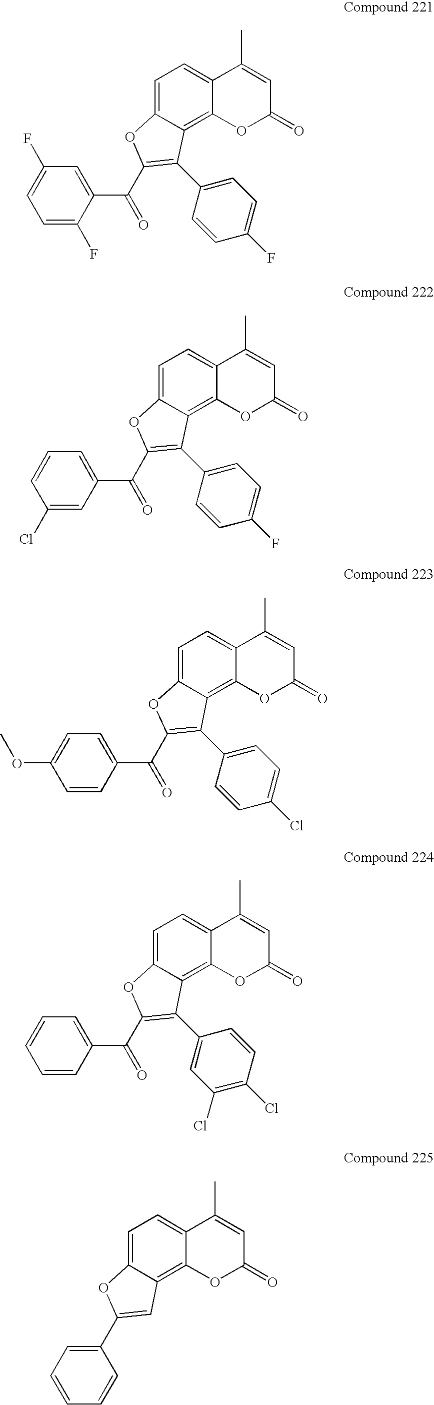

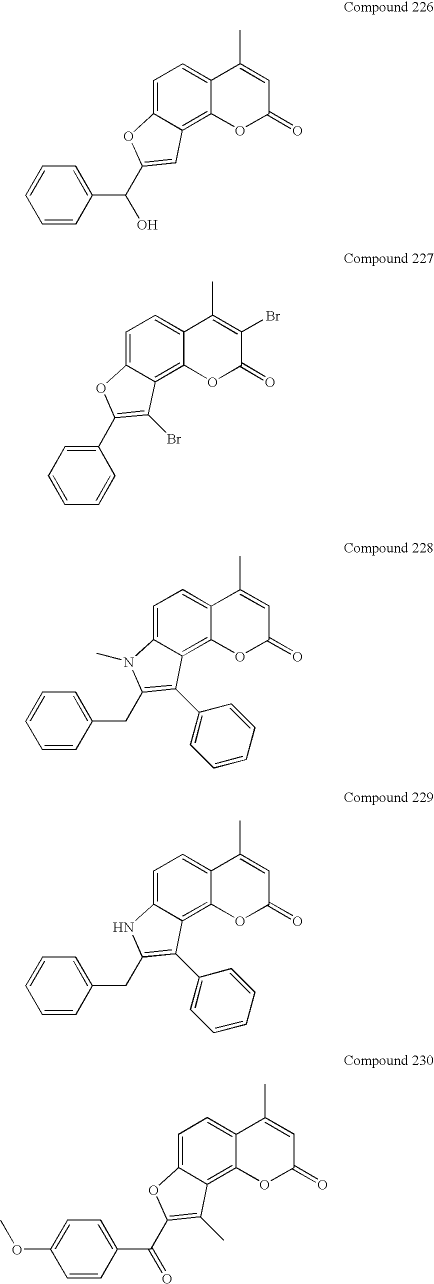

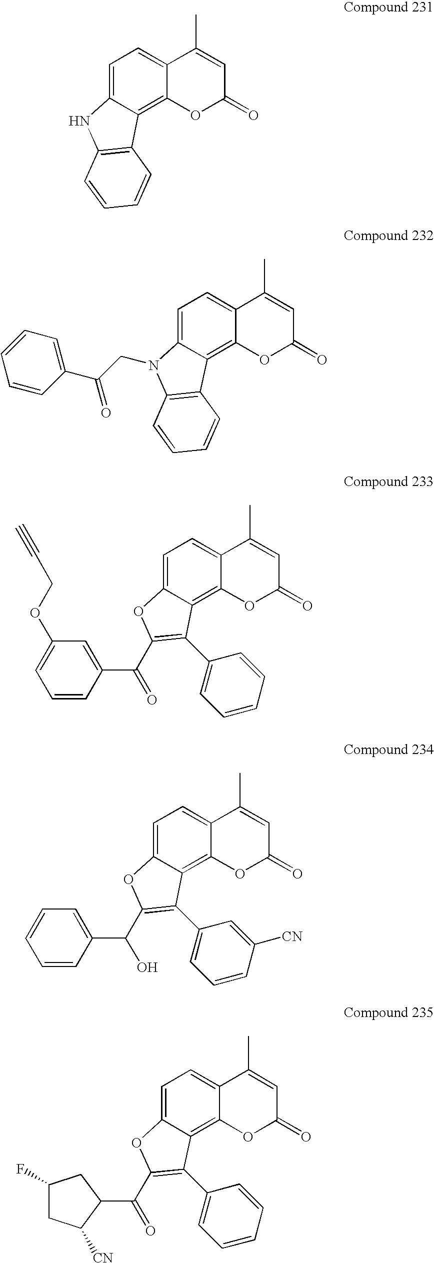

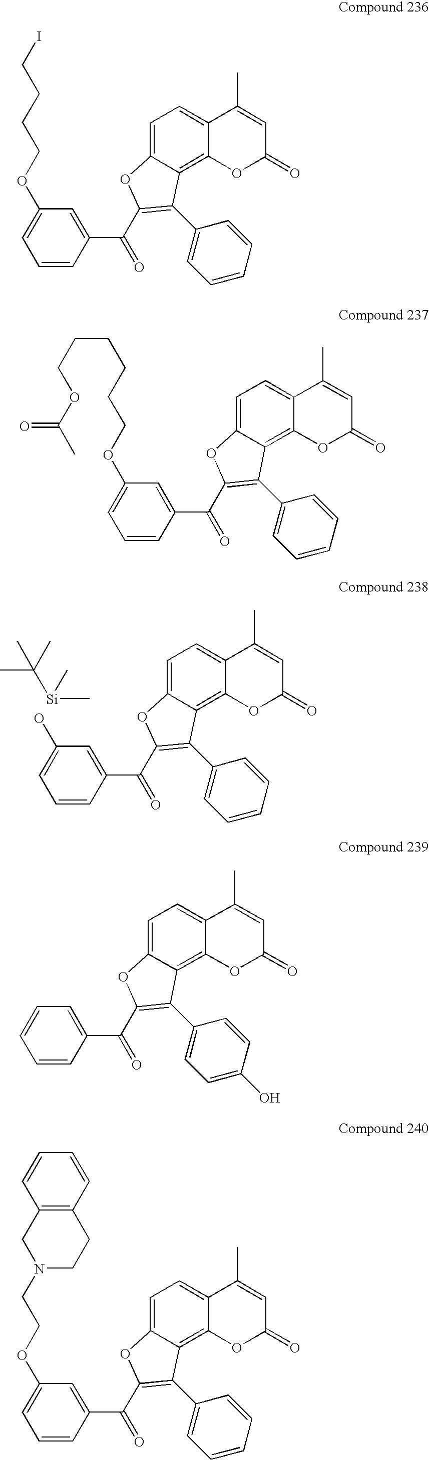

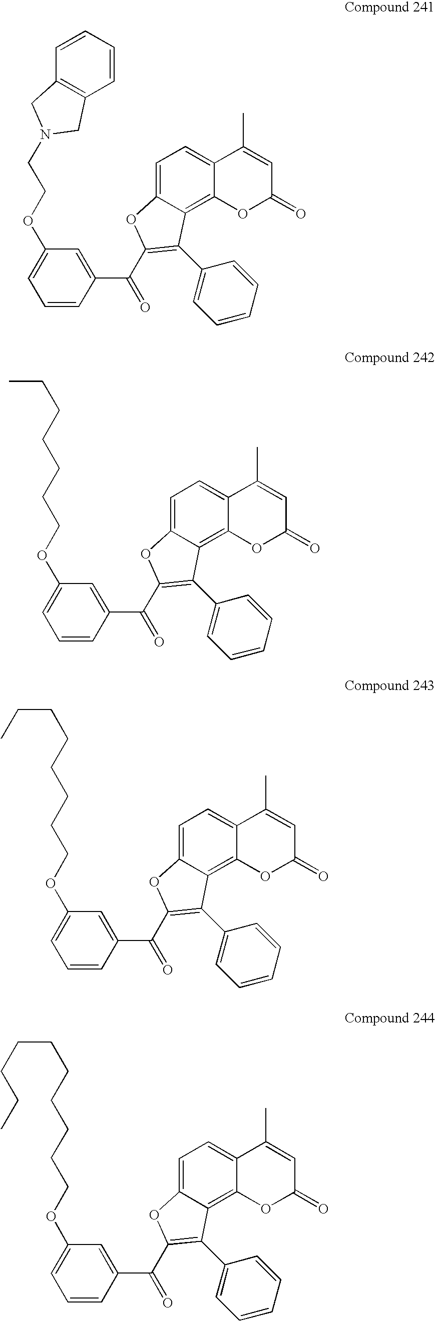

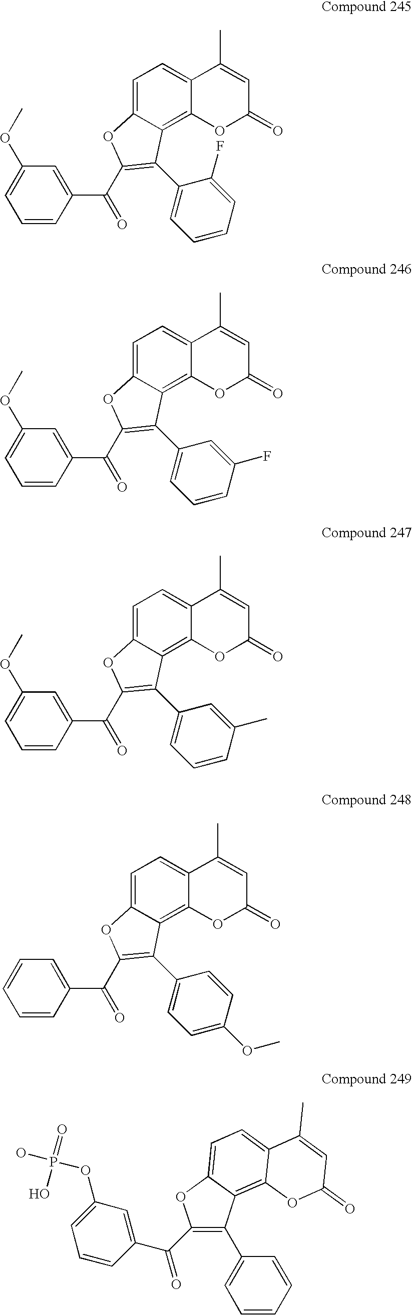

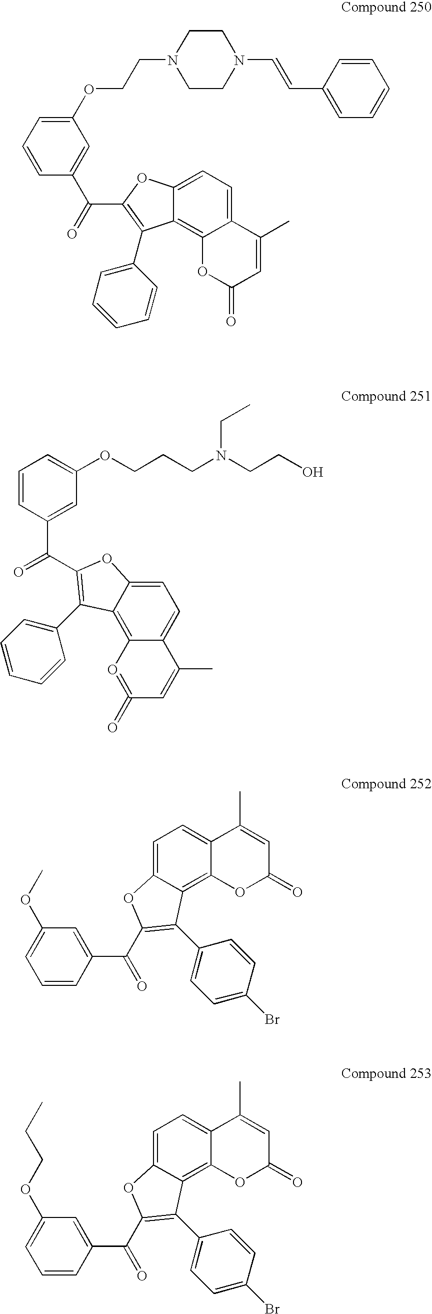

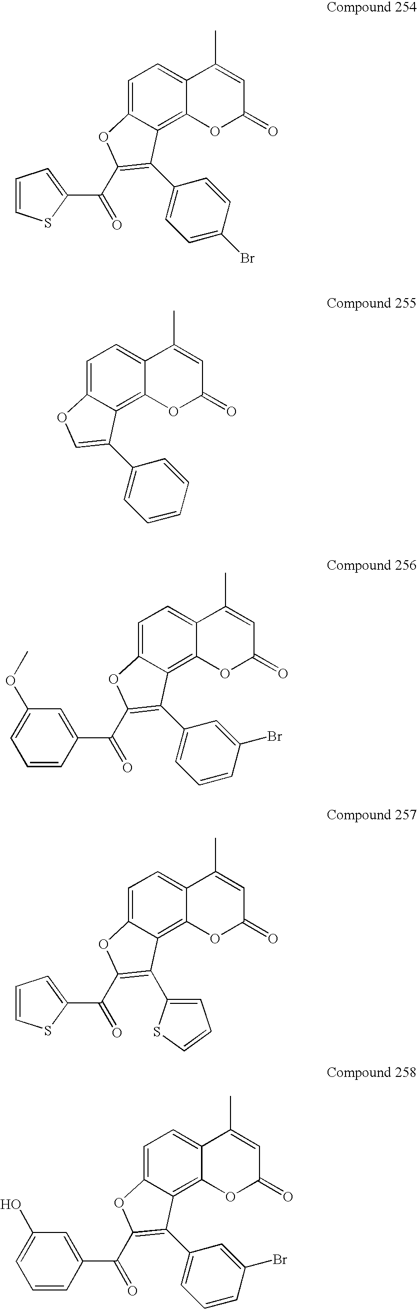

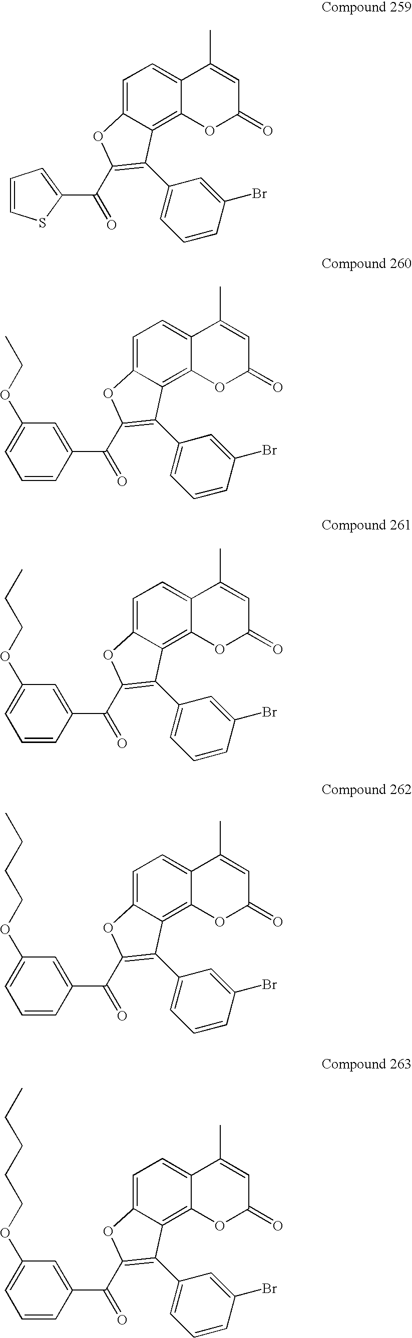

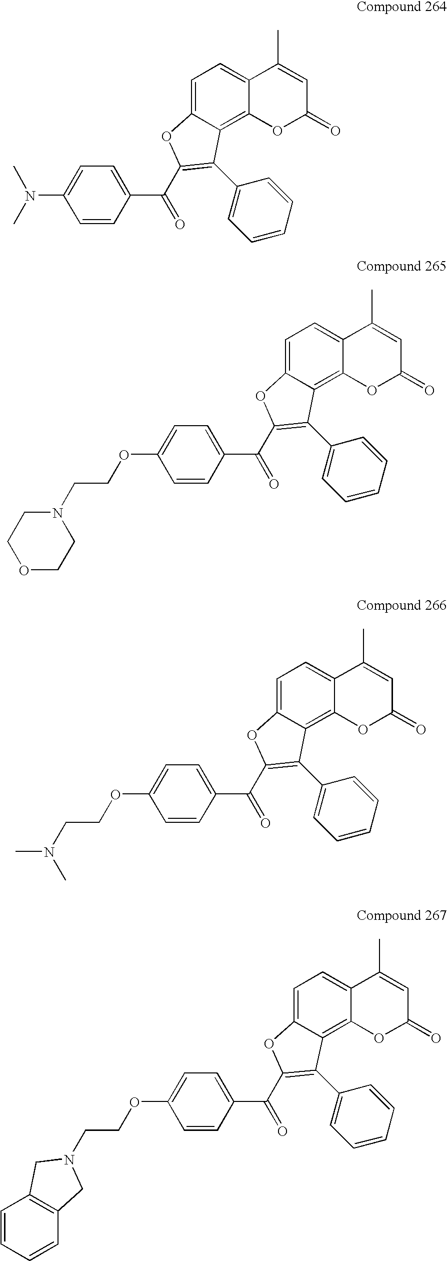

The coumarin compounds described herein can be prepared by conventional chemical transformations (including protecting group methodologies), e.g., those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof. The coumarin compounds can also be synthesized in manners similar to those_described, e.g., in Brubaker et al., J. Med. Chem., 1986, 29, 1094-1099, Limaye, Chem. Ber., 1934, 67, 12-14, and Geetanjali et al., Indian J. Chem. Sect. B, 1983, 22, 164-165, with necessary modifications as recognized by those skilled in the art.

The route shown in Scheme 1 exemplifies synthesis of the coumarin compounds of the present invention. Triethylamine is added to a solution of 7-hydroxy-4-methyl-chromen-2-one (i) and a benzoyl chloride (ii) in THF at room temperature. The reaction mixture is stirred at room temperature overnight and filtered. The filtrate is concentrated to afford a 7-benzoyloxy-4-methyl-coumarin (iii). A mixture of compound (iii) and finely powdered aluminum chloride is heated at 170° C. for 2 hours to afford an 8-benzoyl-7-hydroxy-4-methyl-chromen-2-one (iv). A mixture of compound (iv), 2-bromoacetophenone (v), and K₂CO₃ in CH₃CN is refluxed overnight. The reaction mixture is filtered and the filtrate is concentrated. The residue is purified by column chromatography to afford a pure 8-benzoyl-4-methyl-9-phenyl-furo[2,3-h]chromen-2-one (vi).

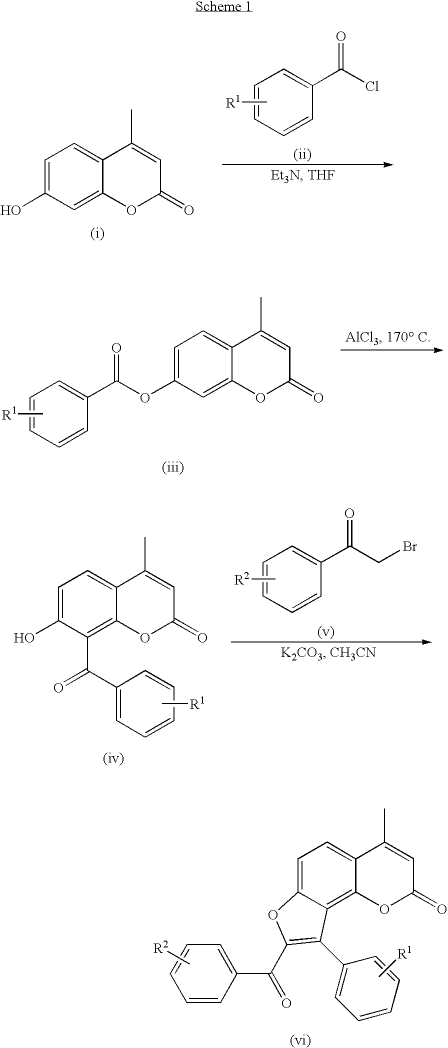

A coumarin compound thus synthesized can be further purified by flash column chromatography, high performance liquid chromatography, crystallization, or any other suitable methods.

The coumarin compounds mentioned herein may contain a non-aromatic double bond and one or more asymmetric centers. Thus, they can occur as racemates and racemic mixtures, single enantiomers, individual diastereomers, diastereomeric mixtures, and cis- or trans-isomeric forms. All such isomeric forms are contemplated.

The viral infection that can be treated by the method of the invention includes infections caused by various viruses such as DNA viruses (e.g., Adenoviridae, Herpesviridae, Poxyiridae, and Parvoviridae); RNA viruses (e.g., Enteroviruses, SARS, influenza, and hepatitis C); and reverse transcribing viruses (e.g., Human immunodeficiency virus).

The coumarin compounds described herein can be administered in conjunction with another therapeutic agent for treating a viral infection such as influenza and AIDS. Examples of the other therapeutic agents include but are not limited to protease inhibitors (e.g., nafamostat, camostat, gabexate, epsilon-aminocapronic acid and aprotinin), fusion inhibitors (e.g., BMY-27709, CL 61917, and CL 62554), M2 proton channel blockers (e.g., Amantadine and Rimantadine), polymerase inhibitors (e.g., 2-deoxy-2′fluoroguanosides (2′-fluoroGuo), 6-fluoro-3-hydroxy-2-pyrazinecarboxamide (T-705), T-705-4-ribofuranosyl-5′-triphosphate (T-705RTP)), endonuclease inhibitors (e.g., L-735,822 and flutimide), kinase inhibitors (e.g., U0126 (a MEK inhibitor), PD098059 (a MEK-specific inhibitor), PD-184352/CT-1040 (a MEK inhibitor), PD 0325901 (a MEK inhibitor), ARRY-142886/AZD-6244 (a MEK1 and MEK2 inhibitor)), neuraminidase inhibitors (e.g., Zanamivir (Relenza), Oseltamivir (Tamiflu), Peramivir and ABT-675 (A-315675)), all of which were described in Hsieh et al., Current Pharmaceutical Design, 2007, 13, 3531-3542. Other examples of antiviral drugs that can be administered in conjunction with the coumarin compounds described herein include, but are not limited to, reverse transcriptase inhibitor (e.g., Abacavir, Adefovir, Delavirdine, Didanosine, Efavirenz, Emtricitabine, Lamivudine, Nevirapine, Stavudine, Tenofovir, Tenofovir disoproxil, and Zalcitabine) Aciclovir, Acyclovir, protease inhibitor (e.g., Amprenavir, Indinavir, Nelfinavir, Ritonavir, and Saquinavir), Arbidol, Atazanavir, Atripla, Boceprevir, Cidofovir, Combivir, Darunavir, Docosanol, Edoxudine, entry inhibitors (e.g., Enfuvirtide and Maraviroc), Entecavir, Famciclovir, Fomivirsen, Fosamprenavir, Foscarnet, Fosfonet, Ganciclovir, Ibacitabine, Immunovir, Idoxuridine, Imiquimod, Inosine, integrase inhibitor (e.g., Raltegravir), interferons (e.g., types I, II, and III), Lopinavir, Loviride, Moroxydine, Nexavir, nucleoside analogues (e.g., Aciclovir), Penciclovir, Pleconaril, Podophyllotoxin, Ribavirin, Tipranavir, Trifluridine, Trizivir, Tromantadine, Truvada, Valaciclovir (Valtrex), Valganciclovir, Vicriviroc, Vidarabine, Viramidine, and Zidovudine.

To practice the method of this invention, the above-described pharmaceutical composition can be administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques.

A sterile injectable composition, e.g., a sterile injectable aqueous or oleaginous suspension, can be formulated according to techniques known in the art using suitable dispersing or wetting agents (such as Tween 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium (e.g., synthetic mono- or diglycerides). Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purposes of formulation.

A composition for oral administration can be any orally acceptable dosage form including, but not limited to, capsules, tablets, emulsions and aqueous suspensions, dispersions and solutions. In the case of tablets for oral use, carriers that are commonly used include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added. A nasal aerosol or inhalation composition can be prepared according to techniques well known in the art of pharmaceutical formulation. A coumarin compound-containing composition can also be administered in the form of suppositories for rectal administration.

The carrier in the pharmaceutical composition must be “acceptable” in the sense of being compatible with the active ingredient of the formulation (and preferably, capable of stabilizing it) and not deleterious to the subject to be treated. One or more solubilizing agents (e.g., cyclodextrins) which form more soluble complexes with the coumarin compounds can be utilized as pharmaceutical carriers for delivery of the active compounds. Examples of other carriers include colloidal silicon dioxide, magnesium stearate, sodium lauryl sulfate, and D&C Yellow # 10.

Suitable in vitro assays can be used to preliminarily evaluate the efficacy of the coumarin compounds in inhibiting the cytopathic effect induced by a virus. The compounds can further be examined for their efficacy in treating an infection with the virus. For example, a compound can be administered to an animal (e.g., a mouse model) having a viral infection and its therapeutic effects are then assessed. Based on the results, an appropriate dosage range and administration route can also be determined.

Without further elaboration, it is believed that the above description has adequately enabled the present invention. The following examples are, therefore, to be construed as merely illustrative, and not limitative of the remainder of the disclosure in any way whatsoever. All of the publications cited herein are hereby incorporated by reference in their entirety.

### Example 1

### Synthesis of 8-benzoyl-4-methyl-9-phenyl-furo[2,3-h]chromen-2-one (Compound 1)

7-benzoyloxy-4-methyl-coumarin: To a solution of 7-hydroxy-4-methyl-chromen-2-one (0.5210 g, 3.0 mmol) and benzoyl chloride (0.4844 g, 0.4 mL, d=1.211 g/mL, 3.4 mmol) in THF (40 mL) was added Et₃N (1 mL) at room temperature. The reaction mixture was stirred at room temperature overnight and filtered. The filtrate was concentrated to give the crude product 7-benzoyloxy-4-methyl-coumarin.

¹H NMR δ 8.230-7.210 (m, 8H), 6.297 (d, J=0.9 Hz, 1H), 2.466 (d, J=0.9 Hz, 3H).

8-benzoyloxy-7-hydroxy-4-methyl-chromen-2-one: A mixture of 7-benzoyloxy-4-methyl-coumarin (0.28 g, 1 mmol) and finely powdered aluminum chloride (0.40 g, 3 mmol) was heated at 170° C. for 2 hours. After the mixture was cooled to room temperature, ice and dilute hydrochloric acid were added. The mixture was extracted with ethyl acetate. The ethyl acetate solution was washed successively with dilute acid, water, and sat. NaHCO₃ (aq). The organic layer was concentrated to provide 8-benzoyloxy-7-hydroxy-4-methyl-chromen-2-one (0.21 g) as a grayish material.

¹H NMR (300 MHz, CDCl₃): δ 10.85 (br, OH), 7.717-7.657 (m, 3H), 7.637˜7.573 (m, 1H), 7.501-7.429 (m, 2H), 7.021 (d, J=9 Hz, 1H), 6.072 (s, 1H), 2.415 (d, J=0.6 Hz, 3H)

8-benzoyl-4-methyl-9-phenyl-furo[2,3-h]chromen-2-one: A mixture of 8-benzoyl-7-hydroxy-4-methyl-chromen-2-one (30 mg, 0.1 mmol), 2-bromoacetophenone (22 mg, 0.11 mmol), and K₂CO₃ (143 mg, 1.03 mmol) in CH₃CN (5 mL) was refluxed overnight. The reaction mixture was filtered and the filtrate was concentrated. The residue was purified by silica gel column chromatography (hexane/ethyl acetate=3/1 then hexane/ethyl acetate=1/1, R_{f}=0.33 hexane/ethyl acetate=1/1) to provide 8-benzoyl-4-methyl-9-phenyl-furo[2,3-h]chromen-2-one as a yellow solid (71% yield).

¹H NMR (300 MHz, CDCl₃): δ 7.78-7.32 (m, 12H), 6.24 (d, J=0.9 Hz, 1H), 2.49 (d, J=1.2 Hz, 3H). ¹³C NMR (100 MHz, CDCl₃): δ 185.5, 159.4, 156.4, 152.8, 149.9, 148.1, 136.5, 132.8, 130.6, 129.6, 128.7, 128.6, 128.0, 127.7, 124.2, 116.3, 115.3, 113.5, 108.9, 19.5.

### Example 2

### Example 2: Syntheses of Compounds 2-4, 6, 8-12, 14, 16-22, 26, 30-92, 94-98, 100-102, 105-107, 109-122, 127-151, 153-161, 165, 166, 170-191, and 193-267

Compounds 2-4, 6, 8-12, 16-22, 26, 30-92, 94-98, 100-102, 105-107, 109-122, 127-151, 153-161, 165, 166, 170-191, and 193-267 were prepared in a manner similar to that described in Example 1. ¹H NMR, ¹³C NMR, IR, or MS data of these compounds are listed in Table 1 below:

TABLE 1

| Cpd# | Analytical Data |
|---|---|
| 2 | ¹H NMR (400 MHz, CDCl₃): δ 7.78-7.74 (m, 3H), 7.58-7.41 (m, 5H), 7.32-7.26 (m, |
| | 5H), 6.35 (d, J = 9.6 Hz, 1H). ¹³C NMR (125 MHz, CDCl₃): δ 185.4, 159.4, 156.6, |
| | 150.5, 148.2, 144.0, 136.6, 132.9, 130.6, 129.7, 129.5, 128.8, 128.4, 128.1, 127.8, |
| | 127.5, 116.4, 114.9, 114.3, 109.4. HRMS (M⁺): Calcd. for C₂₄H₁₄O₄ 366.0892, found |
| | 366.0876. |
| 3 | ¹H NMR (600 MHz, CDCl₃): δ 7.76-7.75 (m, 2H), 7.57 (d, J = 1.1 Hz, 1H), |
| | 7.51-7.41 (m, 5H), 7.31-7.26 (m, 5H), 2.15 (s, 3H). ¹³C NMR (150 MHz, CDCl₃): δ 185.5, 160.9, |
| | 155.8, 149.1, 147.9, 139.7, 136.6, 132.8, 130.6, 129.6, 128.7, 128.4, 128.0, 127.7, |
| | 126.8, 124.1, 116.1, 114.9, 109.1, 17.7. HRMS (M⁺): Calcd. for C₂₅H₁₆O₄ 380.1049, |
| | found 380.1039. |
| 4 | ¹H NMR (400 MHz, CDCl₃): δ 7.97 (d, J = 10.0 Hz, 1H), 7.77-7.75 (m, 2H), |
| | 7.74-7.38 (m, 4H), 7.32-7.26 (m, 5H), 6.38 (d, J = 10.0 Hz, 1H), 2.66 (s, 3H). |
| 6 | ¹H NMR (600 MHz, CDCl₃): δ 7.76-7.72 (m, 1H), 7.73 (d, J = 9.0 Hz, 1H), 7.54 (d, J = 9.0 Hz, |
| | 1H), 7.46-7.41 (m, 2H), 7.30-7.26 (m, 5H), 2.46 (s, 3H), 2.17 (s, 3H). |
| | ¹³C NMR (150 MHz, CDCl₃): δ 185.6, 160.7, 155.7, 148.1, 148.0, 146.3, 136.7, 132.8, |
| | 130.7, 129.8, 129.7, 128.8, 128.7, 128.0, 127.7, 124.2, 120.8, 116.1, 115.9, 108.7, 15.9, |
| | 13.4. HRMS (M⁺): Calcd. for C₂₆H₁₈O₄ 394.1205, found 394.1194. |
| 8 | ¹H NMR (300 MHz, CDCl₃): δ 8.02-7.96 (m, 1H), 7.81-7.78 (m, 2H), 7.54-7.42 (m, |
| | 4H), 7.34-7.26 (m, 4H), 6.09 (s, 1H), 2.63 (s, 3H), 2.47 (s, 3H). |
| 9 | ¹H NMR (600 MHz, CDCl₃): δ 7.76-7.73 (m, 2H), 7.54 (d, J = 8.9 Hz, 1H), |
| | 7.45-7.42 (m, 3H), 7.30-7.27 (m, 5H), 6.23 (q, J = 2.0 Hz, 1H), 2.85 (qd, J = 7.4, 2.0 Hz, 2H), |
| | 1.33 (t, J = 7.4 Hz, 3H). ¹³C NMR (150 MHz, CDCl₃): δ 185.5, 159.8, 157.8, 156.3, |
| | 150.1, 148.1, 136.6, 132.8, 130.6, 129.6, 128.7, 128.0, 127.7, 123.8, 116.5, 114.6, |
| | 111.5, 108.9, 25.4, 12.2. HRMS (M⁺): Calcd. for C₂₆H₁₈O₄ 394.1205, found 394.1205. |
| 10 | ¹H NMR (600 MHz, CDCl₃): δ 10.09 (s, 1H), 8.77 (d, J = 9.1 Hz, 1H), 7.77 (d, J = 7.2 Hz, |
| | 1H), 7.63 (d, J = 9.1 Hz, 1H), 7.46-7.44 (m, 4H), 7.33-7.27 (m, 5H), 6.83 (s, 1H). |
| | ¹³C NMR (150 MHz, CDCl₃): δ 191.5 (CH), 185.3 (C), 159.0 (C), 156.8 (C), 144.1 (C), |
| | 136.4 (C), 133.1 (CH), 130.5 (CH), 129.7 (CH × 2, C), 129.4 (C), 128.9 (CH), 128.2 (C), |
| | 128.1 (CH × 2), 127.9 (CH × 2), 125.5 (CH), 124.2 (CH), 116.4 (C), 113.6 (C), |
| | 110.1 (CH), 109.0 (C). EIMS m/z (relative intensity): 394 (M⁺, 27), 380 (26), 379 (36), |
| | 235 (45), 221 (49), 133 (73), 119 (82), 105 (100), 97 (56), 85 (74). HRMS Calcd. for |
| | C₂₅H₁₄O₄ 394.3757, found 394.0847. IR (neat): 2920, 2851, 1734, 1709, 1653, 1600, |
| | 1446, 1356, 1239, 1078 cm⁻¹. |
| 11 | ¹H NMR (600 MHz, CDCl₃): δ 8.03-8.00 (m, 1H), 7.77-7.75 (m, 1H), 7.62 (d, J = 9.1 Hz, |
| | 1H), 7.45-7.43 (m, 3H), 7.32-7.28 (m, 5H), 6.86 (s, 1H), 6.72 (s, 1H). |
| | ¹³C NMR (150 MHz, CDCl₃): δ 185.3 (C), 158.7 (C), 156.5 (C), 156.5 (C), 150.9 (C), |
| | 150.2 (C), 148.4 (C), 136.5 (C), 134.2 (q, J = 90.2 Hz, C), 133.0 (CH), 130.6 (CH × 2), |
| | 129.7 (CH × 2), 129.4 (C), 128.9 (CH), 128.1 (CH × 2), 127.8 (CH × 2), 124.1 (CH), |
| | 116.9 (C), 112.8 (CH), 110.5 (C), 109.4 (CH). |
| 12 | ¹H NMR (600 MHz, CDCl₃): δ 7.77-7.75 (m, 2H), 7.58 (d, J = 9.0 Hz, 1H), |
| | 7.53-7.52 (m, 3H), 7.50-7.47 (m, 3H), 7.50-7.42 (m, 3H), 7.33-7.32 (m, 3H), 7.29-7.27 (m, 2H), |
| | 6.31 (s, 1H). ¹³C NMR (100 MHz, CDCl₃): δ 185.5, 159.4, 156.6, 156.3, 150.7, 148.2, |
| | 136.6, 135.7, 132.9, 130.7, 129.8, 129.7, 128.9, 128.8, 128.3, 128.1, 127.8, 126.7, |
| | 116.6, 114.5, 114.2, 113.6, 108.9. HRMS (M⁺): Calcd. for C₃₀H₁₈O₄ 442.1205, found |
| | 442.1206. |
| 14 | ¹H NMR (300 MHz, CDCl₃): δ 7.77-7.72 (m, 3H), 7.53-7.41 (m, 4H), 7.31-7.31 (m, |
| | 5H), 6.20 (s, 1H), 2.76 (t, J = 7.6 Hz, 2H), 1.76-1.71 (m, 2H), 1.05 (t, J = 7.6 Hz, 3H). |
| | ¹³C NMR (75 MHz, CDCl₃): δ 185.3, 159.5, 156.1, 150.0, 147.9, 136.4, 132.7, 130.5, |
| | 129.5, 128.6, 127.9, 127.6, 123.9, 116.3, 114.5, 112.2, 108.7, 34.4, 21.3, 13.7. |
| 16 | ¹H NMR (600 MHz, CDCl₃): δ 7.83~7.81 (m, 2H), 7.49-7.45 (m, 4H), 7.33-7.30 (m, |
| | 5H), 6.22 (s, 1H), 6.11-6.05 (m, 1H), 5.24-5.20 (m, 2H), 3.76-3.75 (m, 2H), 2.47 (s, |
| | 3H). ¹³C NMR (150 MHz, CDCl₃): δ 185.1 (C), 159.7 (C), 154.9 (C), 152.8 (C), |
| | 148.6 (C), 148.0 (C), 136.7 (C), 134.8 (CH), 132.9 (CH), 130.6 (CH × 2), 129.8 (CH × 2), |
| | 129.7 (C), 129.0 (C), 128.7 (CH), 128.0 (CH × 2), 127.7 (CH × 2), 123.3 (CH), 121.4 (C), |
| | 117.4 (CH₂), 116.1 (C), 115.4 (C), 113.5 (CH), 33.5 (CH₂), 19.6 (CH₃). EIMS m/z (relative |
| | intensity): 420 (M⁺, 100), 391 (10), 334 (53), 320 (70), 305 (11). HRMS Calcd. for |
| | C₂₈H₂₀O₄ 420.1362, found 420.1356. IR (neat): 2980, 2918, 1730, 1650, 1552, 1585, |
| | 1494, 1474, 1446, 1348, 1226, 1227, 1179, 1126 cm⁻¹. |
| 17 | ¹H NMR (400 MHz, CDCl₃): δ 7.81-7.80 (m, 2H), 7.49-7.43 (m, 4H), 7.35-7.28 (m, |
| | 5H), 6.21 (s, 1H), 2.97 (t, J = 7.6 Hz, 2H), 2.47 (s, 3H), 1.84 (tq, J = 7.2, 7.6 Hz, 2H), |
| | 1.03 (t, J = 7.2 Hz, 3H). ¹³C NMR (100 MHz, CDCl₃): δ 185.3 (C), 159.7 (C), |
| | 155.2 (C), 152.8 (C), 148.3 (C), 147.9 (C), 136.8 (C), 132.8 (CH), 130.6 (CH × 2), 129.8 (C), |
| | 129.7 (CH × 2), 128.9 (C), 128.7 (CH), 128.1 (CH × 2), 127.7 (CH × 2), 123.8 (C), |
| | 123.2 (CH), 116.0 (C), 115.3 (C), 113.5 (CH), 31.5 (CH₂), 23.0 (CH₂), 19.6 (CH₃), |
| | 13.9 (CH₃). EIMS m/z (relative intensity): 422 (M⁺, 5), 336 (26), 322 (100), 307 (40), |
| | 293 (95), 245 (41), 215 (93), 187 (68), 132 (15), 105 (63), 91 (42), 77 (54). HRMS Calcd. |
| | for C₂₈H₂₂O₄ 422.1518, found 422.1499. IR (neat): 2957, 2927, 2868, 1731, 1651, |
| | 1584, 1553, 1490, 1446, 1420, 1373, 1348, 1265, 1230, 1179, 1082 cm⁻¹. |
| 18 | ¹H NMR (600 MHz, CDCl₃): δ 7.78-7.76 (m, 2H), 7.60 (s, 1H), 7.47-7.39 (m, 3H), |
| | 7.32-7.24 (m, 5H), 6.22 (q, J = 1.0 Hz, 1H), 3.40 (dd, J = 4.5, 14.7 Hz, 1H), |
| | 3.37-3.35 (m, 1H), 3.11 (dd, J = 6.1, 14.7 Hz, 1H), 2.86 (dd, J = 3.3, 7.8 Hz, 1H), 2.64 (dd, J = 2.5, |
| | 4.8 Hz, 1H), 2.48 (d, J = 1.0 Hz, 3H). ¹³C NMR (150 MHz, CDCl₃): δ 185.4 (C), |
| | 159.6 (C), 155.1 (C), 152.8 (C), 148.9 (C), 148.0 (C), 136.6 (C), 132.9 (CH), 130.6 (CH × 2), |
| | 129.7 (CH × 2), 129.5 (C), 129.0 (C), 128.8 (CH), 128.0 (CH × 2), 127.8 (CH × 2), |
| | 124.3 (CH), 118.5 (C), 116.1 (C), 115.5 (C), 113.6 (CH), 51.1 (CH), 47.1 (CH₂), |
| | 32.4 (CH₂), 19.6 (CH₃). EIMS m/z (relative intensity): 436 (M⁺, 39), 395 (100), 380 (72), |
| | 208 (30), 204 (42), 191 (54), 172 (67), 144 (57), 105 (40), 77 (23). HRMS Calcd. for |
| | C₂₈H₂₀O₅ 436.1311, found 436.1294. IR (neat): 2985, 2952, 2918, 1731, 1651, 1553, |
| | 1492, 1474, 1446, 1367, 1349, 1267, 1227, 1181, 1124 cm⁻¹. |
| 19 | LCMS [M + 1]⁺: 471.1 |
| 20 | ¹H NMR (600 MHz, CDCl₃): δ 7.76~7.75 (m, 2H), 7.56-7.53 (m, 2H), 7.47-7.38 (m, |
| | 3H), 7.31-7.26 (m, 5H), 3.14 (t, J = 7.1 Hz, 2H), 2.89 (t, J = 7.5 Hz, 2H), 2.22-2.19 (tt, J = 7.1, |
| | 7.5 Hz, 2H). ¹³C NMR (150 MHz, CDCl₃): δ 185.6 (C), 158.8 (C), 156.3 (C), |
| | 156.1 (C), 150.2 (C), 148.0 (C), 142.2 (C), 136.7 (C), 132.8 (CH), 130.7 (CH × 2), |
| | 129.7 (CH × 2), 128.7 (CH), 128.2 (C), 128.1 (CH × 2), 127.8 (CH × 2), 126.2 (C), 124.5 (CH), |
| | 116.4 (C), 114.3 (C), 108.9 (CH), 32.7 (CH₂), 30.7 (CH₂), 22.4 (CH₂). EIMS m/z |
| | (relative intensity) 406 (M⁺, 100), 377 (16). HRMS Calcd. for C₂₇H₁₈O₄ 406.1205, |
| | found 406.1202. IR (neat): 2957, 2851, 1727, 1650, 1600, 1549, 1490, 1479, 1447, |
| | 1369, 1283, 1237, 1071 cm⁻¹. |
| 21 | ¹H NMR (600 MHz, CDCl₃): δ 7.76 (dd, J = 8.4, 1.2 Hz, 2H), 7.69 (d, J = 8.9 Hz, 1H), |
| | 7.53 (d, J = 8.9 Hz, 1H), 7.46-7.41 (m, 3H), 7.30-7.26 (m, 5H), 2.85-2.83 (m, 2H), |
| | 2.54-2.52 (m, 2H), 1.88-1.86 (m, 2H), 1.79-1.78 (m, 2H). ¹³C NMR (150 MHz, CDCl₃): |
| | δ 185.6, 160.4, 155.7, 147.9, 147.3, 136.7, 132.8, 130.1, 129.9, 129.8, 129.7, 128.8, |
| | 128.7, 128.0, 127.7, 123.1, 122.4, 116.2, 115.5, 108.6, 25.9, 24.0, 21.5. HRMS (M⁺): |
| | Calcd. for C₂₈H₂₀O₄ 420.1263, found 420.1265. |
| 22 | ¹H NMR (600 MHz, CDCl₃): δ 8.33 (d, J = 7.8 Hz, 1H), 8.18 (d, J = 8.8 Hz, 1H), |
| | 8.1 (d, J = 8.2 Hz, 1H), 7.84-7.81 (m, 1H), 7.78 (dd, J = 7.1, 1.3 Hz, 2H), 7.61 (dd, J = 8.7, |
| | 0.7 Hz, 1H), 7.54 (t, J = 7.5 Hz, 1H), 7.50-7.48 (m, 2H), 7.43 (t, J = 7.3 Hz, 1H), |
| | 7.33-7.27 (m, 5H). ¹³C NMR (150 MHz, CDCl₃): δ 185.6 (C), 159.9 (C), 155.8 (C), |
| | 148.1 (C), 147.0 (C), 136.7 (C), 135.0 (CH), 134.2 (C), 132.8 (CH), 132.5 (C), 130.7 (CH × 2), |
| | 130.6 (CH), 129.9 (C), 129.7 (CH), 128.7 (CH), 128.5 (CH), 128.1 (CH × 2), 127.7 (CH × 2), |
| | 122.7 (CH), 121.7 (CH), 120.4 (C), 117.0 (C), 113.1 (C), 109.2 (CH). EIMS m/z |
| | (relative intensity) 415 (M+, 4), 316 (75), 315 (100), 239 (28), 105 (23), 77 (13). |
| | HRMS Calcd. for C28H16O4 416.1049, found 416.1033. IR (neat): 2924, 2851, 1737, |
| | 1650, 1599, 1552, 1488, 1446, 1353, 1310, 1237, 1096 cm⁻¹. |
| 26 | ¹H NMR (600 MHz, CDCl₃): δ 7.78 (d, J = 8.6 Hz, 1H), 7.65 (d, J = 8.6 Hz, 1H), |
| | 7.58 (dd, J = 1.0, 7.2 Hz, 2H), 7.34-7.29 (m, 3H), 7.19-7.16 (m, 5H), 6.21 (q, J = 0.8 Hz, |
| | 1H), 2.47 (d, J = 0.8 Hz, 3H). ¹³C NMR (150 MHz, CDCl₃): δ 191.4 (C), 159.0 (C), |
| | 152.6 (C), 151.1 (C), 144.1 (C), 140.4 (C), 139.3 (C), 137.0 (C), 134.2 (C), 132.7 (CH), |
| | 130.6 (CH × 2), 129.5 (CH × 2), 128.3 (CH), 127.9 (CH × 2), 127.4 (CH × 2), 126.7 (C), |
| | 112.4 (CH), 118.5 (CH), 116.1 (C), 114.2 (CH), 19.5 (CH₃). EIMS m/z (relative |
| | intensity): 396 (M⁺, 8), 367 (4), 302 (4), 287 (6), 252 (5), 125 (7), 84 (100). |
| | HRMS Calcd. for C₂₅H₁₆O₃S 396.082, found 396.0815. IR (neat): 2924, 2854, 1737, |
| | 1653, 1590, 1508, 1450, 1378, 1330, 1264, 1175, 1108 cm⁻¹. |
| 30 | ¹H NMR (300 MHz, CDCl₃): δ 7.70 (d, J = 9.0 Hz, 1H), 7.55 (d, J = 8.7 Hz, 1H), |
| | 7.37 (dd, J = 7.2, 1.8 Hz, 1H), 7.33-7.29 (m, 2H), 7.26-7.17 (m, 4H), 6.86 (td, J = 7.4, 0.7 Hz, |
| | 1H), 6.52 (d, J = 8.4 Hz, 1H), 6.21 (d, J = 1.2 Hz, 1H), 3.57 (s, 3H), 2.48 (d, J = 1.2 Hz, |
| | 3H). ¹³C NMR (100 MHz, CDCl₃): δ 185.5, 159.5, 157.3, 156.4, 152.9, 150.1, |
| | 149.1, 132.8, 130.3, 123.0, 129.3, 128.5, 128.4, 127.9, 127.1, 124.2, 120.2, 116.6, |
| | 115.1, 113.3, 110.5, 109.0, 55.2, 19.5. |
| 31 | ¹H NMR (300 MHz, CDCl₃): δ 7.73 (d, J = 8.7 Hz, 1H), 7.57 (d, J = 9.0 Hz, 1H), |
| | 7.50-7.47 (m, 2H), 7.40-7.28 (m, 5H), 7.20 (t, J = 8.4 Hz, 1H), 7.01-6.97 (m, 1H), 6.24 (d, J = 1.2 Hz, |
| | 1H), 3.76 (s, 3H), 2.49 (d, J = 1.2 Hz, 3H). ¹³C NMR (75 MHz, CDCl₃): |
| | δ 185.2, 159.5, 159.2, 156.4, 152.8, 149.9, 148.1, 137.8, 130.6, 129.7, 129.1, 128.8, |
| | 127.7, 124.2, 122.5, 119.7, 116.3, 115.3, 113.7, 113.5, 108.9, 55.3, 19.5. |
| 32 | ¹H NMR (300 MHz, CDCl₃): δ 7.82 (dd, J = 10.2, 2.4 Hz, 2H), 7.70 (dd, J = 9.0, 1.5 Hz, |
| | 1H), 7.56-7.47 (m, 3H), 7.37-7.32 (m, 3H), 6.79 (dd, J = 8.7, 2.7 Hz, 2H), 6.30 (d, J = 1.5, |
| | 0.9 Hz, 1H), 3.81 (s, 3H), 2.48 (d, J = 0.9 Hz, 3H). |
| 33 | ¹H NMR (400 MHz, CDCl₃): δ 7.77 (d, J = 8.4 Hz, 1H), 7.58 (dd, J = 8.4, 0.4 Hz, 1H), |
| | 7.48-7.46 (m, 2H), 7.34 (t, J = 2.4 Hz, 4H), 7.24-7.23 (m, 1H), 7.16 (t, J = 8.0 Hz, 1H), |
| | 6.94 (ddd, J = 8.0, 2.4, 0.8 Hz, 1H), 6.26 (s, 1H), 2.51 (s, 3H). LCMS [M + 1]⁺: 397.1. |
| 34 | ¹H NMR (300 MHz, CDCl₃): δ 7.71-7.58 (m, 3H), 7.51 (d, J = 8.7 Hz, 1H), |
| | 7.44-7.41 (m, 2H), 7.28-7.25 (m, 3H), 6.69-6.66 (m, 2H), 6.15 (d, J = 1.2 Hz, 1H), 2.43 (d, J = 1.2 Hz, |
| | 3H). ¹³C NMR (75 MHz, CDCl₃): δ 182.6, 161.7, 158.6, 155.2, 152.6, 148.5, |
| | 147.6, 131.5, 129.7, 129.2, 127.4, 126.7, 125.7, 123.1, 115.0, 114.3, 112.1, 108.0, 18.6. |
| 35 | ¹H NMR (300 MHz, CDCl₃): δ 7.77-7.21 (m, 11H), 6.23 (d, J = 0.9 Hz, 1H), 2.49 (d, J = 0.9 Hz, |
| | 3H). |
| 36 | ¹H NMR (300 MHz, CDCl₃): δ 7.95-7.29 (m, 11H), 6.26 (d, J = 1.2 Hz, 1H), 2.51 (d, J = 1.2 Hz, |
| | 3H). ¹³C NMR (75 MHz, CDCl₃): δ 184.2, 159.3, 156.6, 152.8, 150.1, 147.5, |
| | 137.2, 132.5, 130.8, 130.7, 130.4, 129.8, 129.2, 129.14, 129.06, 128.8, 127.9, 126.45, |
| | 126.40, 124.8, 116.3, 115.5, 113.7, 109.0, 19.5. |
| 37 | ¹H NMR (300 MHz, CDCl₃): δ 7.84-7.30 (m, 11H), 6.25 (s, 1H), 2.49 (s, 3H). |
| | ¹³C NMR (75 MHz, CDCl₃): δ 184.3, 159.3, 156.5, 152.8, 150.1, 147.6, 139.6, 134.0, |
| | 133.5, 130.6, 129.9, 129.7, 129.3, 129.1, 127.8, 125.2, 125.01, 124.95, 124.9, 124.8, |
| | 116.3, 115.5, 113.7, 108.9, 19.5. |
| 38 | ¹H NMR (300 MHz, CDCl₃): δ 7.80-7.08 (m, 11H), 6.26 (d, J = 0.9 Hz, 1H), 2.50 (d, J = 0.6 Hz, |
| | 3H). |
| 39 | ¹H NMR (300 MHz, CDCl₃): δ 7.75-6.93 (m, 11H), 6.24 (d, J = 1.2 Hz, 1H), 2.42 (s, |
| | 3H), 2.18 (s, 3H). |
| 40 | ¹H NMR (300 MHz, CDCl₃): δ 7.75-7.18 (m, 11H), 6.26 (s, 1H), 2.51 (d, J = 0.6 Hz, |
| | 3H), 2.25 (s, 3H). |
| 41 | ¹H NMR (300 MHz, CDCl₃): δ 7.73-7.69 (m, 3H), 7.55 (d, J = 8.7 Hz, 1H), |
| | 7.51-7.47 (m, 2H), 7.36-7.32 (m, 3H), 7.11 (d, J = 8.1 Hz, 2H), 6.24 (d, J = 0.9 Hz, 1H), 2.49 (d, J = 0.6 Hz, |
| | 3H), 2.35 (s, 3H). ¹³C NMR (100 MHz, CDCl₃): δ 185.1, 159.5, 156.3, |
| | 152.8, 149.9, 148.3, 143.9, 133.9, 130.6, 129.9, 129.7, 128.8, 128.7, 128.6, 128.2, |
| | 127.7, 124.0, 116.3, 115.3, 113.5, 108.9, 21.6, 19.5. |
| 42 | ¹H NMR (300 MHz, CDCl₃): δ 7.98-7.95 (m, 2H), 7.78 (d, J = 8.7 Hz, 1H), |
| | 7.70-7.67 (m, 1H), 7.60 (d, J = 9.0 Hz, 1H), 7.46-7.40 (m, 3H), 7.36-7.32 (m, 3H), 6.26 (d, J = 1.2 Hz, |
| | 1H), 2.51 (d, J = 0.9 Hz, 3H). |
| 43 | ¹H NMR (300 MHz, CDCl₃): δ 7.79-7.74 (m, 3H), 7.58-7.53 (m, 3H), 7.42-7.39 (m, |
| | 2H), 7.37-7.29 (m, 3H), 6.25 (d, J = 1.2H, 1H), 2.49 (s, 3H). |
| 44 | ¹H NMR (300 MHz, CDCl₃): δ 7.74 (d, J = 9.0 Hz, 1H), 7.56 (d, J = 9.0 Hz, 1H), |
| | 7.51-6.78 (m, 9H), 6.24 (d, J = 1.2 Hz, 1H), 2.49 (d, J = 1.2 Hz, 3H). |
| 45 | ¹H NMR (300 MHz, CDCl₃): δ 7.77 (d, J = 8.7 Hz, 1H), 7.57 (d, J = 8.7 Hz, 1H), |
| | 7.57-7.11 (m, 9H), 6.26 (d, J = 1.2 Hz, 1H), 2.51 (d, J = 1.2 Hz, 3H). LCMS [M + 1]⁺: 415.0. |
| 46 | ¹H NMR (300 MHz, CDCl₃): δ 7.84-7.79 (m, 2H), 7.74 (d, J = 9.0 Hz, 1H), 7.58 (d, J = 8.7 Hz, |
| | 1H), 7.48-7.45 (m, 2H), 7.36-7.32 (m, 3H), 6.96-6.94 (m, 2H), 6.26 (d, J = 0.9 Hz, |
| | 1H), 2.50 (d, J = 0.6 Hz, 3H). ¹³C NMR (100 MHz, CDCl₃): δ 183.9, 166.7, 164.2, |
| | 159.4, 156.5, 152.8, 150.0, 147.9, 132.9, 132.8, 132.4, 132.3, 130.6, 129.5, 128.9, |
| | 128.8, 127.8, 124.3, 116.3, 115.41, 115.39, 115.2, 113.6, 108.9, 19.6. |
| 47 | ¹H NMR (600 MHz, CDCl₃): δ 7.71 (d, J = 8.8 Hz, 1H), 7.53 (dd, J = 1.8, 8.8 Hz, 1H), |
| | 7.36-7.24 (m, 2H), 7.27-7.07 (m, 7H), 6.20 (s, 1H), 2.45 (s, 3H). ¹³C NMR (150 MHz, |
| | CDCl₃): δ 184.5 (C), 159.3 (C), 156.6 (C), 152.8 (C), 150.2 (C), 147.9 (C), 137.4 (C), |
| | 131.7 (CH), 130.3 (C), 130.2 (CH × 2), 129.72 (CH), 129.65 (CH), 128.9 (C), |
| | 128.7 (CH), 127.4 (CH × 2), 126.4 (CH), 125.0 (CH), 116.7 (C), 115.3 (C), 113.5 (CH), |
| | 109.0 (CH), 29.6 (C), 19.5 (CH₃). EIMS m/z (relative intensity) 416 (14), 414 (M⁺, 34), |
| | 84 (100). HRMS Calcd. for C₂₅H₁₅ClO₄ 414.0659, found 414.0666. IR (neat): 2923, |
| | 2853, 1734, 1657, 1628, 1603, 1555, 1493, 1471, 1434, 1378, 1357, 1272, 1180, 1152, |
| | 1080 cm⁻¹. |
| 48 | ¹H NMR (300 MHz, CDCl₃): δ 7.64-7.37 (m, 11H), 6.23 (d, J = 1.2 Hz, 1H), 2.50 (d, J = 1.2 Hz, |
| | 3H). ¹³C NMR (75 MHz, CDCl₃): δ 184.0, 159.3, 156.5, 152.8, 150.0, 147.6, |
| | 138.1, 134.2, 132.7, 130.5, 129.6, 129.5, 129.4, 129.0, 127.8, 127.6, 124.6, 116.3, |
| | 115.4, 113.6, 109.0, 19.5. |
| 49 | ¹H NMR (300 MHz, CDCl₃): δ 7.76-7.71 (m, 3H), 7.57 (d, J = 8.7 Hz, 1H), |
| | 7.48-7.44 (m, 2H), 7.38-7.32 (m, 3H), 7.29-7.24 (m, 2H), 6.25 (d, J = 1.2 Hz, 1H), 2.50 (d, J = 1.2 Hz, |
| | 3H). ¹³C NMR (100 MHz, CDCl₃): δ 184.1, 159.4, 156.4, 152.8, 150.0, 147.8, |
| | 139.2, 134.9, 131.0, 130.6, 129.4, 129.1, 128.9, 128.4, 127.9, 124.4, 116.3, 115.4, |
| | 113.6, 108.9, 19.5. |
| 50 | ¹H NMR (300 MHz, CDCl₃): δ 7.75-7.34 (m, 11H), 6.25 (d, J = 0.6 Hz, 1H), 2.50 (d, J = 0.6 Hz, |
| | 3H). ¹³C NMR (75 MHz, CDCl₃): δ 184.2, 159.4, 156.4, 152.8, 150.0, 147.8, |
| | 135.3, 131.4, 131.1, 130.6, 129.4, 129.2, 128.9, 128.0, 127.9, 124.5, 116.3, 115.4, |
| | 113.6, 108.9, 19.5. |
| 51 | ¹H NMR (300 MHz, CDCl₃): δ 8.14-8.09 (m, 2H), 7.89-7.85 (m, 2H), 7.79 (d, J = 9.0 Hz, |
| | 1H), 7.59 (d, J = 8.7 Hz, 1H), 7.46-7.43 (m, 2H), 7.36-7.30 (m, 3H), 6.27 (d, J = 0.9 Hz, |
| | 1H), 2.52 (d, J = 1.2 Hz, 3H). |
| 52 | ¹H NMR (300 MHz, CDCl₃): δ 7.87-7.83 (m, 2H), 7.73 (d, J = 8.7 Hz, 1H), |
| | 7.59-7.30 (m, 13H), 6.25 (d, J = 1.2 Hz, 1H), 2.49 (d, J = 1.2 Hz, 3H). ¹³C NMR (100 MHz, |
| | CDCl₃): δ 185.5, 159.5, 157.3, 156.4, 152.7, 150.1, 149.1, 132.8, 130.3, 130.0, 129.3, |
| | 128.5, 128.4, 127.9, 127.1, 124.2, 120.2, 116.6, 115.1, 113.3, 110.5, 109.0, 55.2, 19.5. |
| 53 | ¹H NMR (300 MHz, CDCl₃): δ 8.23 (dd, J = 7.8, 1.8 Hz, 1H), 7.81-7.73 (m, 3H), |
| | 7.61-7.48 (m, 4H), 7.23-6.98 (m, 7H), 6.23 (d, J = 0.9 Hz, 1H), 2.50 (d, J = 0.9 Hz, 3H). |
| 54 | ¹H NMR (300 MHz, CDCl₃): δ 8.31 (d, J = 0.6 Hz, 1H), 7.88-7.25 (m, 13H), 6.25 (d, J = 0.6 Hz, |
| | 1H), 2.49 (d, J = 1.2 Hz, 3H). ¹³C NMR (75 MHz, CDCl₃): δ 185.3, 159.5, |
| | 156.5, 152.8, 149.9, 148.4, 135.3, 133.7, 132.1, 132.0, 130.6, 129.7, 129.4, 128.6, |
| | 128.5, 128.0, 127.8, 127.6, 126.6, 124.9, 124.1, 116.4, 115.3, 113.5, 109.0, 19.5. |
| 55 | ¹H NMR (300 MHz, CDCl₃): δ 7.81-7.44 (m, 4H), 7.72-7.47 (m, 5H), 7.32-7.29 (m, |
| | 3H), 6.25 (d, J = 1.2 Hz, 1H), 2.51 (d, J = 1.2 Hz, 3H), 2.14 (s, 3H). |
| | ¹³C NMR (75 MHz, CDCl₃): δ 184.0, 168.7, 159.7, 156.4, 153.2, 149.8, 148.3, 142.6, |
| | 131.8, 131.3, 130.5, 129.7, 128.7, 128.1, 127.8, 124.1, 118.3, 116.3, 115.4, 113.4, |
| | 109.0, 24.7, 19.6. |
| 56 | ¹H NMR (300 MHz, CDCl₃): δ 7.67 (d, J = 9.0 Hz, 1H), 7.52 (d, J = 8.7 Hz, 1H), |
| | 7.40 (d, J = 8.4 Hz, 1H), 7.37-7.33 (m, 2H), 7.26-7.21 (m, 3H), 6.38 (dd, J = 2.1, 8.4 Hz, |
| | 1H), 6.20 (d, J = 1.2 Hz, 1H), 6.03 (d, J = 2.4 Hz, 1H), 3.76 (s, 3H), 3.52 (s, 3H), |
| | 2.46 (s, 3H). |
| 57 | ¹H NMR (300 MHz, CDCl₃): δ 7.76 (d, J = 9.0 Hz, 1H), 7.56 (d, J = 9.0 Hz, 1H), |
| | 7.37-7.08 (m, 8H), 6.23 (d, J = 1.2 Hz, 1H), 2.49 (d, J = 1.2 Hz, 3H). |
| | ¹³C NMR (75 MHz, CDCl₃): δ 159.3, 156.7, 152.7, 137.3, 135.9, 132.8, 130.6, 130.2, |
| | 129.7, 128.9, 128.8, 127.6, 126.8, 125.1, 115.4, 113.7, 109.0, 19.5. |
| 58 | ¹H NMR (600 MHz, CDCl₃): δ 7.73 (d, J = 8.9 Hz, 1H), 7.59-7.54 (m, 2H), |
| | 7.44-7.43 (m, 3H), 7.35-7.33 (m, 4H), 6.23 (q, J = 1.0 Hz, 1H), 2.48 (d, J = 1.0 Hz, 3H). |
| | ¹³C NMR (150 MHz, CDCl₃): δ 182.9 (C), 159.3 (C), 156.5 (C), 152.7 (C), 150.0 (C), |
| | 147.4 (C), 137.3 (C), 136.1 (C), 132.6 (C), 131.6 (CH), 130.5 (CH × 2), 130.2 (CH), |
| | 129.8 (C), 129.3 (C), 129.1 (CH), 128.5 (CH), 127.9 (CH × 2), 124.8 (CH), 116.3 (C), |
| | 115.5 (C), 113.7 (CH), 108.9 (CH), 19.5 (CH₃). EIMS m/z (relative intensity) 448 (M⁺, |
| | 100), 450 (59), 452 (12), 419 (31), 269 (56), 195 (80), 189 (51), 175 (47), 145 (67), |
| | 75 (62). HRMS Calcd. for C₂₅H₁₄Cl₂O₄ 448.0269, found 448.0269. IR (neat): 1736, |
| | 1649, 1627, 1602, 1553, 1490, 1467, 1443, 1381, 1354, 1267, 1238, 1176, 1152, 1130, |
| | 1079, 1030, 1001 cm⁻¹. |
| 59 | ¹H NMR (300 MHz, CDCl₃): δ 7.72 (d, J = 8.7 Hz, 1H), 7.54 (d, J = 8.7 Hz, 1H), |
| | 7.49 (dd, J = 8.7, 1.8 Hz, 1H), 7.41-7.38 (m, 2H), 7.31-7.27 (m, 3H), 6.79 (td, J = 8.4, 2.4 Hz, |
| | 1H), 6.56-6.49 (m, 1H), 6.22 (s, 1H), 2.47 (s, 3H). |
| 60 | ¹H NMR (600 MHz, CDCl₃): δ 7.74 (d, J = 8.9 Hz, 1H), 7.65-7.62 (m, 1H), |
| | 7.57-7.54 (m, 2H), 7.46-7.44 (m, 2H), 7.36-7.33 (m, 3H), 7.06 (dd, J = 8.4, 17.2 Hz, 1H), 6.24 (q, |
| | J = 1.0 Hz, 1H), 2.49 (d, J = 1.0 Hz, 3H). ¹³C NMR (150 MHz, CDCl₃): δ 182.6 (C), |
| | 159.3 (C), 156.5 (C), 153.3 (dd, J = 256.2, 12.8 Hz, C), 152.7 (C), 150.0 (C), 149.9 (dd, |
| | J = 249.5, 13.1 Hz, C), 147.4 (C), 133.5 (C), 130.5 (CH × 2), 129.5 (C), 129.4 (C), |
| | 129.1 (CH), 127.9 (CH × 2), 126.8 (d, J = 4.1 Hz, CH), 124.69 (CH), 119.0 (d, J = 18.3 Hz, |
| | CH), 117.1 (d, J = 17.7 Hz, CH), 116.3 (C), 115.5 (C), 113.7 (CH), 108.9 (CH), |
| | 19.5 (CH₃). EIMS m/z (relative intensity) 416 (M⁺, 100), 387 (33), 141 (70), 113 (60), |
| | 84 (52), 77 (17). HRMS Calcd. for C₂₅H₁₄F₂O₄ 416.086, found 416.0857. IR (neat): |
| | 1737, 1656, 1650, 1604, 1555, 1514, 1493, 1473, 1430, 1379, 1355, 1289, 1236, 1203, |
| | 1161, 1111, 1079 cm⁻¹. |
| 61 | ¹H NMR (300 MHz, CDCl₃): δ 7.75 (d, J = 8.7 Hz, 1H), 7.56 (d, J = 8.7 Hz, 1H), |
| | 7.44-7.41 (m, 2H), 7.32-7.28 (m, 3H), 7.19-7.13 (m, 1H), 7.04-6.96 (m, 1H), 6.78 (td, J = 8.9, |
| | 4.4 Hz, 1H), 6.24 (d, J = 1.2 Hz, 1H), 2.490 (d, J = 1.2 Hz, 3H). |
| | ¹³C NMR (100 MHz, CDCl₃): δ 180.9, 159.42, 159.38, 159.3, 157.11, 157.08, 156.97, |
| | 156.92, 156.6, 154.62, 154.59, 152.7, 150.2, 147.8, 130.5, 130.4, 129.0, 128.8, 127.55, |
| | 127.46, 127.37, 127.30, 125.1, 120.24, 120.15, 120.0, 119.9, 117.4, 117.3, 117.2, 117.1, |
| | 116.72, 116.68, 116.53, 116.47, 116.43, 115.4, 113.6, 109.0, 19.5. |
| 62 | ¹H NMR (300 MHz, CDCl₃): δ 7.71 (d, J = 9.0 Hz, 1H), 7.53 (d, J = 8.7 Hz, 1H), |
| | 7.40-7.36 (m, 2H), 7.28-7.20 (m, 3H), 7.19 (d, J = 7.5 Hz, 1H), 6.92 (s, 1H), 6.77 (d, J = 8.1 Hz, |
| | 1H), 6.23 (d, J = 1.2 Hz, 1H), 2.48 (d, J = 0.6 Hz, 3H), 2.38 (s, 3H), 2.25 (s, 3H). |
| | ¹³C NMR (100 MHz, CDCl₃): δ 187.4, 159.5, 156.4, 152.8, 150.0, 148.8, 141.6, 137.7, |
| | 134.1, 131.7, 130.3, 129.9, 129.5, 128.7, 128.4, 127.5, 125.7, 124.3, 116.5, 115.2, |
| | 113.5, 109.0, 21.3, 19.9, 19.5. |
| 63 | ¹H NMR (400 MHz, CDCl₃): δ 7.77 (d, J = 6.6 Hz, 1H), 7.85-7.21 (m, 8H), 6.74 (d, J = 6.3 Hz, |
| | 1H), 6.29 (d, J = 0.9 Hz, 1H), 6.02 (s, 2H), 2.50 (d, J = 0.9 Hz, 3H). |
| 64 | ¹H NMR (300 MHz, CDCl₃): δ 7.85-7.32 (m, 8H), 7.76 (d, J = 8.7 Hz, 1H), 6.79 (d, J = 8.7 Hz, |
| | 1H), 6.29 (d, J = 1.2 Hz, 1H), 4.30-4.28 (m, 2H), 4.25-4.22 (m, 2H), 2.50 (d, J = 1.2 Hz, |
| | 3H). |
| 65 | ¹H NMR (300 MHz, CDCl₃): δ 7.89 (br, NH), 7.73 (d, J = 8.7 Hz, 1H), 7.57 (d, J = 8.7 Hz, |
| | 1H), 7.52-7.45 (m, 3H), 7.36-7.34 (m, 3H), 7.24 (d, J = 1.8 Hz, 1H), 6.87 (d, J = 8.4 Hz, |
| | 1H), 6.26 (d, J = 1.2 Hz, 1H), 4.65 (s, 2H), 2.51 (d, J = 0.9 Hz, 3H). |
| 66 | ¹H NMR (300 MHz, CDCl₃): δ 8.50 (t, J = 2.0 Hz, 1H), 8.25 (dd, J = 8.1, 1.2 Hz, 1H), |
| | 8.08 (d, J = 7.8 Hz, 1H), 7.79 (d, J = 8.7 Hz, 1H), 7.62 (d, J = 9.0 Hz, 1H), 7.51 (t, J = 8.0 Hz, |
| | 1H), 7.465-7.42 (m, 2H), 7.31-7.26 (m, 3H), 6.27 (s, 1H), 2.52 (d, J = 0.6 Hz, |
| | 3H). |
| 67 | LCMS [M + 1]+: 396.1. |
| 68 | ¹H NMR (300 MHz, CDCl₃): δ 7.73 (d, J = 8.7 Hz, 1H), 7.78 (d, J = 9.0 Hz, 1H), |
| | 7.50-7.46 (m, 2H), 7.37-7.27 (m, 5H), 7.18 (dd, J = 8.4, 8.4 Hz, 1H), 7.00-6.96 (m, 1H), |
| | 6.25 (d, J = 0.9 Hz, 1H), 3.98 (q, J = 6.9 Hz, 2H), 2.50 (d, J = 1.2 Hz, 3H), 1.40 (t, J = 6.9 Hz, |
| | 3H). |
| | ¹³C NMR (75 MHz, CDCl₃): δ 185.3, 159.5, 158.6, 156.4, 152.9, 149.9, 148.1, 137.7, |
| | 130.6, 129.7, 129.1, 128.8. 128.7, 127.7, 124.2, 122.4, 120.1, 116.3, 115.3, 114.3, |
| | 113.5, 109.0, 63.6, 19.6, 14.7. |
| 69 | ¹H NMR (300 MHz, CDCl₃): δ 7.73 (d, J = 8.7 Hz, 1H), 7.77 (d, J = 8.7 Hz, 1H), |
| | 7.50-7.46 (m, 2H), 7.37-7.26 (m, 5H), 7.21-7.15 (m, 1H), 7.00-6.96 (m, 1H), 6.26 (d, J = 1.2 Hz, |
| | 1H), 3.86 (d, J = 6.6 Hz, 2H), 2.50 (d, J = 1.2 Hz, 3H), 1.81-1.72 (m, 2H), 1.02 (t, J = 7.5 Hz, |
| | 1H). ¹³C NMR (75 MHz, CDCl₃): δ 185.4, 159.5, 158.8, 156.4, 152.9, 149.9, |
| | 148.1, 137.7, 132.3, 130.6, 130.1, 129.7, 129.2, 129.1, 128.7, 128.6, 128.2, 127.9, |
| | 127.7, 124.2, 122.3, 120.1, 120.0, 116.3, 115.3, 114.5, 114.4, 113.502, 109.0, 69.6, |
| | 22.4, 19.6, 10.5. |
| 70 | ¹H NMR (300 MHz, CDCl₃): δ 7.73 (d, J = 9.0 Hz, 1H), 7.58 (d, J = 8.7 Hz, 1H), |
| | 7.49-7.46 (m, 2H), 7.37-7.26 (m, 5H), 7.18 (dd, J = 7.8, 7.8 Hz, 1H), 7.00-6.96 (m, 1H), |
| | 6.25 (d, J = 1.2 Hz, 1H), 3.90 (t, J = 6.9 Hz, 2H), 2.60 (d, J = 1.2 Hz, 3H), 1.78-1.70 (m, 2H), |
| | 1.51-1.43 (m, 2H), 0.97 (t, J = 7.2 Hz, 3H). ¹³C NMR (75 MHz, CDCl₃): δ 185.4, |
| | 159.5, 158.8, 156.4, 152.9, 149.9, 148.1, 137.7, 130.6, 129.7, 129.1, 128.7, 128.6, |
| | 127.7, 124.2, 122.3, 120.1, 116.3, 115.3, 114.4, 113.5, 109.0, 67.8, 31.1, 19.6, 19.2, |
| | 13.8. |
| 71 | ¹H NMR (300 MHz, CDCl₃): δ 7.73 (d, J = 9.0 Hz, 1H), 7.77 (d, J = 9.0 Hz, 1H), |
| | 7.49-7.46 (m, 2H), 7.37-7.26 (m, 5H), 7.18 (dd, J = 7.8, 7.8 Hz, 1H), 7.00-6.96 (m, 1H), |
| | 6.25 (d, J = 0.9 Hz, 1H), 3.89 (t, J = 6.6 Hz, 2H), 2.50 (s, 3H), 1.78-1.73 (m, 2H), |
| | 1.44-1.36 (m, 4H), 0.93 (d, J = 6.9 Hz, 3H). ¹³C NMR (75 MHz, CDCl₃): δ 185.4, 159.5, 158.8, |
| | 156.4, 152.9, 149.9, 148.1, 137.7, 130.6, 129.6, 129.0, 128.7, 128.6, 127.7, 124.2, |
| | 122.3, 120.1, 116.3, 115.3, 114.3, 113.5, 109.0, 68.1, 28.8, 28.1, 22.4, 19.6, 14.0. |
| 72 | ¹H NMR (300 MHz, CDCl₃): δ 7.69 (d, J = 9.0 Hz, 1H), 7.52 (d, J = 9.0 Hz, 1H), |
| | 7.49-7.46 (m, 2H), 7.37-7.26 (m, 5H), 7.19-7.14 (m, 1H), 6.99-6.95 (m, 1H), 6.21 (d, J = 0.9 Hz, |
| | 1H), 3.88 (t, J = 6.6 Hz, 2H), 2.45 (d, J = 0.9 Hz, 1H), 1.77-1.72 (m, 2H), |
| | 1.44-1.25 (m, 6H), 0.90 (d, J = 6.6 Hz, 1H). ¹³C NMR (75 MHz, CDCl₃): δ 185.1, 159.3, 158.7, |
| | 156.3, 152.8, 149.7, 148.0, 137.6, 130.5, 129.6, 129.0, 128.6, 128.4, 127.6, 124.1, |
| | 122.1, 119.9, 116.1, 115.2, 114.3, 113.3, 108.8, 68.0, 31.4, 28.9, 25.5, 22.4, 19.4, 13.9. |
| 73 | ¹H NMR (300 MHz, CDCl₃): δ 7.75 (d, J = 9.0 Hz, 1H), 7.59 (d, J = 9.0 Hz, 1H), |
| | 7.50-7.45 (m, 2H), 7.42 (dt, J = 7.8, 1.2 Hz, 1H), 7.36-7.27 (m, 5H), 7.21 (t, J = 8.1 Hz, 1H), |
| | 7.02 (ddd, J = 8.1, 2.4, 0.9 Hz, 1H), 6.27 (d, J = 1.2 Hz, 1H), 4.18 (t, J = 6.0 Hz, 2H), |
| | 3.79 (t, J = 6.0 Hz, 2H), 2.51 (d, J = 1.2 Hz, 3H). |
| 74 | ¹H NMR (300 MHz, CDCl₃): δ 7.74 (d, J = 9.0 Hz, 1H), 7.59 (d, J = 9.0 Hz, 1H), |
| | 7.50-7.46 (m, 2H), 7.39 (d, J = 7.5 Hz, 1H), 7.34~7.32 (m, 3H), 7.28-7.26 (m, 1H), 7.20 (t, J = 8.1 Hz, |
| | 1H), 6.99 (dd, J = 7.2, 2.1 Hz, 1H), 6.26 (d, J = 1.2 Hz, 1H), 4.06 (t, J = 6.0 Hz, |
| | 2H), 3.73 (t, J = 6.0 Hz, 2H), 2.52 (d, J = 1.2 Hz, 3H), 2.21 (quin, J = 6.0 Hz, 2H). |
| 75 | ¹H NMR (300 MHz, CDCl₃): δ 7.75 (d, J = 8.7 Hz, 1H), 7.59 (d, J = 8.7 Hz, 1H), |
| | 7.50-7.47 (m, 2H), 7.37 (dt, J = 7.8, 1.2 Hz, 1H), 7.35-7.30 (m, 3H), 7.28-7.24 (m, 1H), |
| | 7.19 (t, J = 7.8 Hz, 1H), 6.98 (dd, J = 7.2, 2.7 Hz, 1H), 6.26 (s, 1H), 3.94 (t, J = 6.0 Hz, 2H), |
| | 3.62 (t, J = 6.0 Hz, 2H), 2.15 (d, J = 1.2 Hz, 3H), 1.98~1.90 (m, 4H). |
| 76 | ¹H NMR (300 MHz, CDCl₃): δ 7.74 (d, J = 8.7 Hz, 1H), 7.58 (d, J = 8.7 Hz, 1H), |
| | 7.50-7.43 (m, 2H), 7.38 (dt, J = 7.8, 1.2 Hz, 1H), 7.34-7.30 (m, 4H), 7.22 (t, J = 8.4 Hz, 1H), |
| | 7.01 (dd, J = 8.4, 2.7 Hz, 1H), 6.26 (d, J = 1.2 Hz, 1H), 6.02 (dddd, J = 17.4, 10.5, 5.4, |
| | 5.4 Hz, 1H), 5.39 (ddd, J = 17.1, 3.0, 1.8 Hz, 1H), 5.30 (ddd, J = 10.5, 3.0, 1.5 Hz, 1H), |
| | 4.49 (ddd, J = 5.7, 1.5, 1.5 Hz, 2H), 2.51 (s, 3H). |
| 77 | ¹H NMR (300 MHz, CDCl₃): δ 7.74 (d, J = 9.0 Hz, 1H), 7.58 (d, J = 9.0 Hz, 1H), |
| | 7.49-7.46 (m, 2H), 7.38-7.27 (m, 5H), 7.18 (t, J = 7.8 Hz, 1H), 6.99 (dd, J = 8.1, 2.7 Hz, 1H), |
| | 6.26 (d, J = 0.9 Hz, 1H), 5.93-5.81 (m, 1H), 5.16 (dd, J = 17.1, 1.5 Hz, 1H), 5.11 (dd, J = 10.5, |
| | 1.2 Hz, 1H), 3.95 (t, J = 6.9 Hz, 2H), 2.51 (t, J = 6.9 Hz, 2H), 2.51 (s, 3H). |
| 78 | ¹H NMR (300 MHz, CDCl₃): δ 7.73 (d, J = 9.0 Hz, 1H), 7.57 (d, J = 9.0 Hz, 1H), |
| | 7.50-7.46 (m, 2H), 7.37-7.26 (m, 5H), 7.18 (t, J = 8.1 Hz, 1H), 7.00-6.97 (dd, J = 8.1, 2.7 Hz, |
| | 1H), 6.25 (s, 1H), 5.91-5.78 (m, 1H), 5.10-4.99 (m, 1H), 3.91 (t, J = 6.0 Hz, 2H), |
| | 2.50 (s, 3H), 2.22 (q, J = 6.6 Hz, 2H), 1.86 (quin, J = 6.6 Hz, 2H). |
| 79 | ¹H NMR (300 MHz, CDCl₃): δ 7.73 (d, J = 9.0 Hz, 1H), 7.58 (d, J = 9.0 Hz, 1H), |
| | 7.50-7.45 (m, 2H), 7.37-7.27 (m, 5H), 7.18 (t, J = 8.1 Hz, 1H), 6.98 (ddd, J = 8.1, 1.8, 0.9 Hz, |
| | 1H), 6.25 (s, 1H), 5.90-5.76 (m, 1H), 5.08-4.96 (m, 2H), 4.90 (t, J = 6.6 Hz, 2H), |
| | 2.50 (s, 3H), 2.12 (q, J = 7.5 Hz, 2H), 1.78 (quin, J = 6.6 Hz, 2H), 1.60 (quin, J = 7.5 Hz, |
| | 2H). |
| 80 | ¹H NMR (300 MHz, CDCl₃): δ 7.74 (d, J = 8.7 Hz, 1H) 7.56 (d, J = 8.7 Hz, 1H), |
| | 7.50-7.47 (m, 2H), 7.38 (dt, J = 8.1, 1.2 Hz, 1H), 7.35-7.29 (m, 4H), 7.20 (t, J = 8.1 Hz, 1H), |
| | 7.00 (ddd, J = 8.1, 2.7, 1.2 Hz, 1H), 6.27 (d, J = 1.2 Hz, 1H), 4.06 (t, J = 5.7 Hz, 2H), |
| | 3.74 (t, J = 4.8 Hz, 2H), 2.79 (t, J = 5.7 Hz, 2H), 2.57 (t, J = 4.8 Hz, 2H), 2.51 (d, J = 1.2 Hz, |
| | 3H). |
| 81 | ¹H NMR (300 MHz, CDCl₃): δ 7.74 (d, J = 8.7 Hz, 1H), 7.58 (d, J = 8.7 Hz, 1H), |
| | 7.50-7.46 (m, 2H), 7.38~7.24 (m, 5H), 7.18 (t, J = 8.4 Hz, 1H), 6.89 (dd, J = 8.4, 1.8 Hz, |
| | 1H), 6.26 (d, J = 1.2 Hz, 1H), 3.97 (t, J = 6.0 Hz, 2H), 3.72 (t, J = 4.8 Hz, 4H), 2.51 (d, |
| | J = 1.2 Hz, 3H), 2.58~2.40 (m, 6H), 1.97 (quin, J = 7.8 Hz, 2H). |
| 82 | ¹H NMR (300 MHz, CDCl₃): δ 7.74 (d, J = 9 Hz, 1H), 7.58 (d, J = 9.0 Hz, 1H), |
| | 7.50-7.47 (m, 2H), 7.38-7.32 (m, 4H), 7.28-7.26 (m, 1H), 7.18 (t, J = 8.1 Hz, 1H), 6.98 (ddd, |
| | J = 8.1, 2.7, 0.9 Hz, 1H), 6.26 (d, J = 1.2 Hz, 1H), 3.93 (t, J = 6.0 Hz, 2H), 3.72 (t, J = 4.5 Hz, |
| | 4H), 2.51 (d, J = 1.2 Hz, 3H), 2.46 (t, J = 7.5 Hz, 4H), 2.40 (t, J = 7.5 Hz, 2H), |
| | 1.82-1.63 (m, 4H). |
| 83 | ¹H NMR (300 MHz, CDCl₃): δ 7.74 (d, J = 9.0 Hz, 1H), 7.59 (d, J = 9.0 Hz, 1H), |
| | 7.50-7.46 (m, 2H), 7.38-7.29 (m, 5H), 7.19 (t, J = 8.1 Hz, 1H), 7.00 (dt, J = 8.1, 2.7 Hz, 1H), |
| | 6.26 (d, J = 1.2 Hz, 1H), 4.06 (t, J = 6.3 Hz, 2H), 3.77 (t, J = 6.3 Hz, 2H), 2.51 (d, J = 1.2 Hz, |
| | 3H). |
| 84 | ¹H NMR (300 MHz, CDCl₃): δ 7.74 (d, J = 8.7 Hz, 1H), 7.59 (d, J = 8.7 Hz, 1H), |
| | 7.49-7.46 (m, 2H), 7.37~7.25 (m, 5H), 7.18 (t, J = 8.4 Hz, 1H), 6.89 (dd, J = 8.4, 2.4 Hz, |
| | 1H), 6.26 (s, 1H), 3.95 (t, J = 6.0 Hz, 2H), 2.51 (s, 3H), 2.60~2.36 (m, 6H), |
| | 1.74-1.40 (m, 6H). |
| 85 | ¹H NMR (300 MHz, CDCl₃): δ 7.75 (d, J = 8.7 Hz, 1H), 7.59 (d, J = 8.7 Hz, 1H), |
| | 7.50-7.47 (m, 2H), 7.38-7.32 (m, 5H), 7.19 (t, J = 8.4 Hz, 1H), 6.98 (dd, J = 8.4, 2.7 Hz, 1H), |
| | 6.26 (d, J = 1.2 Hz, 1H), 3.93 (s, 2H), 2.69-2.55 (m, 6H), 2.51 (s, 3H), 1.79-1.50 (m, |
| | 12H). |
| 86 | ¹H NMR (300 MHz, CDCl₃): δ 7.62 (d, J = 9 Hz, 1H), 7.50~7.21 (m, 9H), 7.11 (dd, J = 8.4, |
| | 2.1 Hz, 1H), 6.26 (s, 1H), 4.47 (t, J = 4.2 Hz, 2H), 3.43 (t, J = 4.2 Hz, 2H), 2.91 (s, |
| | 6H), 2.51 (s, 3H). |
| 87 | ¹H NMR (300 MHz, CDCl₃): δ 7.75 (d, J = 9.0 Hz, 1H), 7.59 (d, J = 9.0 Hz, 1H), |
| | 7.50-7.45 (m, 2H), 7.37-7.24 (m, 5H), 7.18 (t, J = 8.1 Hz, 1H), 6.99 (ddd, J = 8.4, 2.7, 0.9 Hz, |
| | 1H), 6.27 (d, J = 1.2 Hz, 1H), 3.96 (t, J = 6.6 Hz, 2H), 2.52 (d, J = 1.2 Hz, 3H), |
| | 2.50 (t, J = 7.8 Hz, 2H), 2.30 (s, 6H), 1.97 (quin, J = 7.8 Hz, 2H). |
| 88 | ¹H NMR (300 MHz, CDCl₃): δ 7.75 (d, J = 8.7 Hz, 1H), 7.59 (d, J = 8.7 Hz, 1H), |
| | 7.49-7.47 (m, 2H), 7.38-7.32 (m, 5H), 6.99 (t, J = 8.7 Hz, 1H), 6.26 (s, 1H), 3.93 (t, J = 6.0 Hz, |
| | 2H), 2.51 (s, 3H), 2.45 (t, J = 7.2 Hz, 2H), 2.33 (s, 6H), 1.85-1.71 (m, 4H). |
| 89 | ¹H NMR (300 MHz, CDCl₃): δ 7.74 (d, J = 9.0 Hz, 1H), 7.53 (d, J = 9.0 Hz, 1H), |
| | 7.49-7.28 (m, 7H), 7.19 (t, J = 7.5 Hz, 1H), 7.00 (dd, J = 7.5, 2.7 Hz, 1H), 6.26 (d, J = 1.2 Hz, |
| | 1H), 4.05 (t, J = 5.7 Hz, 2H), 2.81 (t, J = 5.7 Hz, 2H), 2.66 (bs, 4H), 2.57 (bs, 4H), |
| | 2.51 (d, J = 1.2 Hz, 3H), 2.35 (s, 3H). |
| 90 | ¹H NMR (300 MHz, CDCl₃): δ 7.75 (d, J = 8.7 Hz, 1H), 7.59 (d, J = 8.7 Hz, 1H), |
| | 7.50~7.47 (m, 2H), 7.38-7.32 (m, 4H), 7.27 (dd, J = 6.9, 2.4 Hz, 1H), 7.18 (t, J = 8.4 Hz, |
| | 1H), 6.99 (dd, J = 8.1, 2.4 Hz, 1H), 6.26 (d, J = 1.2 Hz, 1H), 3.96 (t, J = 6.6 Hz, |
| | 2H), 2.51 (s, 3H), 2.53-2.33 (m, 10H), 2.92 (s, 3H), 1.95 (quin, J = 7.5 Hz, 2H). |
| 91 | ¹H NMR (300 MHz, CDCl₃): δ 7.74 (d, J = 8.7 Hz, 1H), 7.58 (d, J = 8.7 Hz, 1H), |
| | 7.49-7.45 (m, 2H), 7.37-7.27 (m, 5H), 7.18 (t, J = 8.4 Hz, 1H), 6.27 (ddd, J = 8.4, 2.4, 0.6 Hz, |
| | 1H), 6.26 (d, J = 1.2 Hz, 1H), 3.89 (t, J = 6.6 Hz, 2H), 2.50 (d, J = 1.2 Hz, 3H), |
| | 2.60-2.38 (m, 10H), 2.30 (d, J = 1.2 Hz, 3H), 1.75 (quin, J = 6.6 Hz, 4H). |
| 92 | ¹H NMR (300 MHz, CDCl₃): δ 7.74 (d, J = 8.7 Hz, 1H), 7.62-7.56 (m, 4H), |
| | 7.50-7.44 (m, 4H), 6.55 (dd, J = 1.8, 1.8 Hz, 1H), 6.25 (d, J = 1.2 Hz, 1H), 2.50 (d, J = 1.2 Hz, |
| | 3H). |
| | ¹³C NMR (75 MHz, CDCl₃): δ 171.2, 170.7, 159.4, 156.2, 152.7, 151.0, 149.9, 147.6, |
| | 147.1, 130.4, 129.5, 128.8, 127.8, 124.3, 121.2, 116.5, 115.3, 113.6, 112.4, 108.8, 19.6. |
| 94 | ¹H NMR (400 MHz, CDCl₃): δ 7.77-7.72 (m, 1H), 7.61-7.58 (m, 2H), 7.56-7.44 (m, |
| | 3H), 7.22-7.21 (m, 1H), 6.26-6.21 (m, 3H), 2.51 (s, 3H), 2.40 (s, 3H). |
| 95 | ¹H NMR (300 MHz, CDCl₃): δ 8.11-8.10 (m, 1H), 7.76-7.71 (m, 2H), 7.63-7.60 (m, |
| | 3H), 7.58-7.26 (m, 3H), 7.16-7.14 (m, 1H), 6.25 (d, J = 1.2 Hz, 1H), 2.50 (d, J = 1.2 Hz, |
| | 3H). ¹³C NMR (100 MHz, CDCl₃): δ 175.2, 161.69, 160.37, 155.09, 149.83, 149.60, |
| | 142.78, 135.99, 135.26, 134.98, 134.85, 129.59, 129.17, 128.66, 128.55, 126.48, |
| | 126.02, 113.56, 113.46, 113.01, 108.77, 107.84, 25.46. |
| 96 | ¹H NMR (400 MHz, CDCl₃): δ 8.30-8.29 (m, 1H), 7.73 (d, J = 8.8 Hz, 1H), |
| | 7.63-7.62 (m, 1H), 7.58-7.55 (m, 3H), 7.44-7.42 (m, 3H), 7.27-7.25 (m, 1H), 6.23 (d, J = 1.2 Hz, |
| | 1H), 2.48 (s, 3H). ¹³C NMR (100 MHz, CDCl₃): δ 177.3, 159.4, 156.0, 153.0, 152.8, |
| | 149.8, 148.2, 140.3, 134.8, 133.3, 130.4, 129.7, 129.2, 128.8, 128.5, 128.2, 127.9, |
| | 127.8, 127.2, 126.8, 126.6, 126.5, 126.4, 125.6, 124.2, 116.5, 115.3, 113.5, 113.1, |
| | 108.8, 19.5. |
| 97 | ¹H NMR (300 MHz, CDCl₃): δ 7.76 (d, J = 8.7 Hz, 1H), 7.59-7.40 (m, 13H), 6.80 (s, |
| | 1H), 6.26 (d, J = 1.2 Hz, 1H), 2.50 (d, J = 1.2 Hz, 3H). |
| 98 | ¹H NMR (300 MHz, CDCl₃): δ 7.89 (d, J = 3.9 Hz, 1H), 7.76 (d, J = 9.0 Hz, 1H), |
| | 7.62-7.48 (m, 7H), 7.13 (d, J = 3.9 Hz, 1H), 6.26 (d, J = 0.8 Hz, 1H), 2.51 (d, J = 0.8 Hz, |
| | 1H). |
| 100 | ¹H NMR (300 MHz, CDCl₃): δ 7.72 (d, J = 9.0 Hz, 1H), 7.62-7.39 (m, 7H), 6.91 (d, J = 5.1 Hz, |
| | 1H), 6.25 (d, J = 1.2 Hz, 1H), 2.49 (s, 3H), 2.50 (s, 3H). |
| 101 | ¹H NMR (300 MHz, CDCl₃): δ 9.67 (brs, 1H), 7.98-7.93 (m, 1H), 7.75-7.72 (m, 1H), |
| | 7.65-7.42 (m, 6H), 7.10-7.07 (m, 1H), 6.39-6.36 (m, 1H), 6.24 (s, 1H), 2.50 (s, 3H). |
| 102 | LCMS [M + 1]⁺: 384.1. |
| 105 | ¹H NMR (400 MHz, CDCl₃): δ 8.46-7.32 (m, 8H), 6.26 (d, J = 0.6 Hz, 1H), 2.52 (d, J = 0.9 Hz, |
| | 3H). |
| 106 | ¹H NMR (400 MHz, CDCl₃): δ 8.36 (s, 1H), 7.75 (d, J = 8.8 Hz, 1H), 7.64-7.61 (m, |
| | 2H), 7.59-7.42 (m, 9H), 6.26 (d, J = 1.2 Hz, 1H), 2.59 (s, 3H), 2.51 (d, J = 0.8 Hz, 3H). |
| 107 | ¹H NMR (400 MHz, CDCl₃): δ 7.94 (d, J = 2.1 Hz, 1H), 7.78-7.42 (m, 8H), 6.26 (d, J = 0.6 Hz, |
| | 1H), 2.51 (d, J = 0.6 Hz, 3H). |
| 109 | ¹H NMR (300 MHz, CDCl₃): δ 7.69 (d, J = 9.0 Hz, 1H), 7.54-7.46 (m, 6H), 6.21 (d, J = 0.9 Hz, |
| | 1H), 2.48 (d, J = 1.2 Hz, 3H), 2.11 (s, 8H), 1.80 (s, 7H). ¹³C NMR (75 MHz, |
| | CDCl₃): δ 197.2, 159.8, 155.5, 153.0, 150.0, 148.7, 130.6, 130.2, 128.8, 128.0, 124.0, |
| | 116.7, 115.4, 113.8, 108.9, 47.3, 37.8, 36.9, 28.3, 19.8. |
| 110 | ¹H NMR (300 MHz, CDCl₃): δ 7.84-7.49 (m, 8H), 6.27 (d, J = 0.6 Hz, 1H), 4.50 (q, J = 7.2 Hz, |
| | 2H), 2.51 (d, J = 0.6 Hz, 3H), 1.26 (t, J = 7.2 Hz, 3H). ¹³C NMR (75 MHz, |
| | CDCl₃): δ 169.1, 165.9, 159.1, 159.0, 156.7, 156.6, 152.6, 150.2, 145.8, 132.4, 130.3, |
| | 129.6, 128.6, 128.0, 125.8, 116.6, 115.7, 114.0, 110.4, 109.0, 62.7, 29.7, 19.5, 14.1. |
| 111 | ¹H NMR (600 MHz, CDCl₃): δ 7.80-7.79 (m, 2H), 7.63-7.59 (m, 2H), 7.48-7.43 (m, |
| | 8H), 7.33 (s, 1H), 6.25 (s, 1H), 2.49 (s, 3H). ¹³C NMR (150 MHz, CDCl₃): δ 170.2 (C), |
| | 165.4 (C), 162.8 (C), 159.3 (C), 156.6 (C), 152.8 (C), 150.1 (C), 146.3 (C), 131.8 (C), |
| | 130.6 (CH), 130.3 (CH × 2), 129.5 (CH), 129.1 (CH × 2), 128.9 (C), 128.0 (CH × 2), |
| | 126.9 (CH × 2, C), 125.5 (CH), 116.6 (C), 115.6 (C), 113.9 (CH), 109.1 (CH), |
| | 108.2 (CH), 19.6 (CH₃). EIMS m/z (relative intensity): 447 (M⁺, 56), 176 (93), 148 (100), |
| | 91 (31), 84 (53), 77 (46), 71 (22), 57 (38), 51 (43). HRMS Calcd. for C₂₈H₁₇NO₅ |
| | 447.1107, found 447.1106. IR (neat): 2917, 2849, 1736, 1657, 1649, 1599, 1572, 1552, |
| | 1492, 1468, 1421, 1352, 1288, 1250, 1228, 1204, 1167, 1080, 1052 cm⁻¹. |
| 112 | ¹H NMR (300 MHz, CDCl₃): δ 7.81 (d, J = 8.7 Hz, 1H), 7.75 (d, J = 8.4 Hz, 2H), |
| | 7.64-7.60 (m, 3H), 7.52-7.45 (m, 5H), 7.31 (s, 1H), 7.27 (s, J = 0.9 Hz, 1H), 2.59 (s, 3H). |
| | ¹³C NMR (100 MHz, CDCl₃): δ 183.3, 159.2, 156.7, 152.7, 150.2, 147.7, 137.2, 135.8, |
| | 132.7, 130.6, 130.6, 130.2, 129.6, 128.82, 128.80, 127.6, 126.8, 125.2, 116.6, 115.4, |
| | 113.6, 109.0, 19.5. |
| 113 | ¹H NMR (400 MHz, CDCl₃): δ 7.89 (d, J = 2.0 Hz, 1H), 7.80 (d, J = 8.4 Hz, 1H), |
| | 7.66-7.59 (m, 4H), 7.56 (d, J = 8.4 Hz, 1H), 7.49-7.46 (m, 3H), 7.27 (s, 1H), 6.26 (s, 1H), |
| | 2.50 (s, 3H). ¹³C NMR (100 MHz, CDCl₃): δ 169.8 (C), 165.8 (C), 160.9 (C), |
| | 159.1 (C), 156.6 (C), 152.6 (C), 150.2 (C), 146.2 (C), 134.9 (C), 133.5 (C), 132.0 (C), |
| | 131.2 (CH), 130.3 (CH × 2), 129.6 (CH), 128.8 (C), 128.7 (CH), 128.0 (CH × 2), 127.9 (C), |
| | 126.0 (CH), 125.6 (CH), 116.5 (C), 115.7 (C), 113.9 (CH), 109.0 (CH), 107.7 (CH), |
| | 19.5 (CH₃). EIMS m/z (relative intensity): 519 (9), 517 (37), 515 (M⁺, 52), 269 (34), |
| | 195 (52), 176 (54), 148 (62), 86 (61), 84 (100), 75 (53). HRMS Calcd. for |
| | C₂₈H₁₅Cl₂NO₅ 515.0237, found 515.0329. IR (neat): 1736, 1657, 1602, 1555, 1493, |
| | 1468, 1425, 1363, 1287, 1173, 1080, 1031 cm⁻¹. |
| 114 | ¹H NMR (600 MHz, CDCl₃): δ 7.79 (d, J = 8.9 Hz, 1H), 7.67 (d, J = 8.4 Hz, 1H), |
| | 7.60-7.58 (m, 3H), 7.53-7.52 (m, 2H), 7.48-7.46 (m, 3H), 7.36 (dd, J = 1.8, 3.0 Hz, 1H), |
| | 6.25 (s, 1H), 2.49 (s, 3H). ¹³C NMR (150 MHz, CDCl₃): δ 170.1 (C), 165.0 (C), 160.5 (C), |
| | 159.1 (C), 156.6 (C), 152.6 (C), 146.2 (C), 150.2 (C), 137.0 (C), 133.6 (C), 132.0 (C), |
| | 131.8 (CH), 130.4 (CH), 130.3 (CH × 2), 129.5 (CH), 128.8 (C), 128.0 (CH × 2), |
| | 127.8 (C), 125.8 (C), 125.5 (CH), 116.6 (C), 115.6 (C), 113.9 (CH), 111.1 (CH), 109.0 (CH), |
| | 19.6 (CH₃). EIMS m/z (relative intensity): 515 (73), 195 (91), 117 (78), 85 (48), |
| | 71 (70), 57 (100). HRMS Calcd. for C₂₈H₁₅Cl₂NO₅ 515.0327, found 515.0328. IR (neat): |
| | 2923, 2851, 1736, 1656, 1603, 1569, 1551, 1493, 1437, 1384, 1355, 1288, 1230, 1208, |
| | 1171, 1080, 1029 cm⁻¹. |
| 115 | ¹H NMR (300 MHz, CDCl₃): δ 8.25-8.24 (m, 1H), 7.80-7.77 (m, 1H), 7.74-7.67 (m, |
| | 2H), 7.58 (d, J = 9.0 Hz, 1H), 7.42-7.38 (m, 2H), 7.25-7.20 (m, 4H), 6.24 (d, J = 1.2 Hz, |
| | 1H), 2.49 (d, J = 1.2 Hz, 3H). |
| 116 | ¹H NMR (400 MHz, CDCl₃): δ 9.00 (s, 1H), 6.65 (d, J = 4.0 Hz, 1H), 8.05-8.02 (m, |
| | 1H), 7.77 (d, J = 9.0 Hz, 1H), 7.59 (d, J = 9.0 Hz, 1H), 7.49-7.47 (m, 2H), 7.37-7.34 (m, |
| | 3H), 7.27-7.23 (m, 1H), 6.26 (q, J = 1.2 Hz, 1H), 2.50 (d, J = 1.2 Hz, 3H). |
| | ¹³C NMR (100 MHz, CDCl₃): δ 183.5 (C), 159.3 (C), 156.6 (C), 152.9 (CH), 152.7 (C), |
| | 150.6 (CH), 150.1 (C), 147.5 (C), 136.6 (CH), 132.5 (C), 130.7 (CH × 2), 130.0 (C), |
| | 129.2 (C), 129.18 (CH), 128.0 (CH × 2), 124.9 (CH), 123.0 (CH), 116.4 (C), 115.5 (C), |
| | 113.8 (CH), 109.0 (CH), 19.5 (CH₃). EIMS m/z (relative intensity) 381 (M⁺, 7), |
| | 279 (10), 88 (10), 86 (63), 84 (100), 71 (15), 57 (22), 51 (34). HRMS Calcd. for |
| | C₂₄H₁₅ClO₄ 381.1001, found 381.1006. IR (neat): 2924, 2854, 1731, 1650, 1626, 1602, |
| | 1585, 1553, 1492, 1471, 1446, 1416, 1380, 1366, 1263, 1178, 1153, 1080, 1063 cm⁻¹. |
| 117 | ¹H NMR (300 MHz, CDCl₃): δ 8.62-8.60 (m, 1H), 7.78 (d, J = 9.3 Hz, 1H), |
| | 7.59-7.52 (m, 2H), 7.47-7.44 (m, 1H), 7.37-7.27 (m, 2H), 6.25 (d, J = 1.2 Hz, 1H), 2.50 (d, J = 1.2 Hz, |
| | 3H). ¹³C NMR (75 MHz, CDCl₃): δ 184.0, 159.2, 156.5, 152.7, 150.1, 150.0, |
| | 147.1, 143.3, 130.6, 130.5, 129.3, 129.0, 127.8, 125.1, 122.3, 116.2, 115.5, 113.7, |
| | 108.9, 19.5. |
| 118 | ¹H NMR (300 MHz, CDCl₃): δ 7.86-7.83 (m, 2H), 7.61-7.58 (m, 2H), 7.46-7.41 (m, |
| | 2H), 7.26-7.08 (m, H), 6.95 (d, J = 8.7 Hz, 1H), 6.12 (s, 1H), 5.12 (s, 2H), 2.39 (s, 3H). |
| 119 | ¹H NMR (300 MHz, CDCl₃): δ 7.73-7.51 (m, 7H), 6.21 (s, 1H), 2.48 (s, 3H), 2.34 (d, J = 0.8 Hz, |
| | 3H). ¹³C NMR (75 MHz, CDCl₃): δ 188.7, 159.3, 155.9, 152.7, 150.1, 148.2, |
| | 130.3, 130.0, 129.2, 128.4, 128.2, 127.9, 124.6, 117.0, 115.2, 113.5, 108.8, 28.5, 19.4. |
| 120 | ¹H NMR (400 MHz, CDCl₃): δ 7.78-7.76 (m 2H), 7.54-7.50 (m, 2H), 7.40-7.36 (m, |
| | 2H), 7.22-7.19 (m, 1H), 6.11 (d, J = 1.2 Hz, 1H), 3.66 (s, 3H), 2.35 (d, J = 0.8 Hz, 1H). |
| 121 | ¹H NMR (300 MHz, CDCl₃): δ 7.70 (d, J = 8.7 Hz, 1H), 7.57-7.52 (m, 3H), |
| | 7.51-7.47 (m, 3H), 6.22 (d, J = 1.2 Hz, 1H), 4.30 (q, J = 7.1 Hz, 2H), 2.48 (d, J = 1.2 Hz, 3H), |
| | 1.22 (t, J = 7.2 Hz, 3H). |
| 122 | ¹H NMR (300 MHz, CDCl₃): δ 7.70 (d, J = 9.0 Hz, 1H), 7.54-7.46 (m, 6H), 6.22 (d, J = 1.2 Hz, |
| | 1H), 2.48 (d, J = 1.2 Hz, 3H), 1.40 (s, 9H). |
| 127 | ¹H NMR (300 MHz, CDCl₃): δ 7.71 (d, J = 9.0 Hz, 1H), 7.68-7.42 (m, 6H), 6.22 (d, J = 1.2 Hz, |
| | 1H), 3.70 (m, 1H), 2.93-1.07 (m, 10H), 2.59 (s, 3H). |
| 128 | ¹H NMR (300 MHz, CDCl₃): δ 7.65-7.61 (m, 2H), 7.56-7.42 (m, 5H), 6.23 (d, J = 0.9 Hz, |
| | 1H), 2.48 (d, J = 0.9 Hz, 3H). |
| 129 | ¹H NMR (300 MHz, CDCl₃): δ 7.62-7.57 (m, 3H), 7.54-7.43 (m, 4H), 2.67 (s, 3H). |
| 130 | ¹H NMR (300 MHz, CDCl₃): δ 7.65-7.61 (m, 3H), 7.56-7.45 (m, 6H), 7.39-7.29 (m, |
| | 3H), 6.25 (d, J = 0.9 Hz, 1H), 2.44 (d, J = 1.2 Hz, 3H). LCMS [M + 1]⁺: 353.1. |
| 131 | ¹H NMR (300 MHz, CDCl₃): δ 7.53-7.36 (m, 7H), 6.17 (d, J = 1.2 Hz, 1H), 2.77 (t, J = 7.5 Hz, |
| | 2H), 2.44 (d, J = 0.9 Hz, 3H), 1.79 (h, J = 7.5 Hz, 2H), 0.96 (t, J = 7.5 Hz, 3H). |
| 132 | ¹H NMR (300 MHz, CDCl₃): δ 8.05 (d, J = 7.5 Hz, 2H), 7.56 (d, J = 8.7 Hz, 1H), |
| | 7.60-7.39 (m, 6H), 7.06-7.01 (m, 1H), 6.85-6.79 (m, 1H), 6.28 (d, J = 1.2 Hz, 1H), 2.51 (d, J = 1.2 Hz, |
| | 3H). |
| 133 | ¹H NMR (300 MHz, CDCl₃): δ 7.81 (d, J = 7.2 Hz, 2H), 7.76 (d, J = 8.7 Hz, 1H), |
| | 7.59 (d, J = 8.7 Hz, 1H), 7.50 (dd, J = 7.5, 7.5 Hz, 2H), 7.37 (d, J = 8.1 Hz, 1H), |
| | 7.34-7.26 (m, 2H), 7.18 (d, J = 9.9 Hz, 1H), 7.04 (t, J = 8.7 Hz, 1H), 6.27 (s, 1H), 2.51 (s, 3H). |
| 134 | ¹H NMR (300 MHz, CDCl₃): δ 7.80-7.72 (m, 3H), 7.55 (d, J = 9.0 Hz, 1H), |
| | 7.52-7.44 (m, 3H), 7.36-7.31 (m, 2H), 7.04-6.99 (m, 2H), 6.25 (d, J = 1.2 Hz, 1H), 2.49 (d, J = 1.2 Hz, |
| | 3H). ¹³C NMR (75 MHz, CDCl₃): δ 185.2, 164.5, 161.2, 159.4, 156.3, 152.9, |
| | 149.8, 148.1, 136.4, 133.0, 132.5, 132.4, 129.6, 128.1, 127.6, 125.6, 125.5, 124.3, |
| | 115.3, 115.0, 114.7, 113.5, 108.9, 19.5. |
| 135 | ¹H NMR (400 MHz, CDCl₃): δ 7.85 (d, J = 6.8 Hz, 2H), 7.74 (d, J = 8.8 Hz, 1H), |
| | 7.59 (d, J = 8.8 Hz, 1H), 7.49 (t, J = 7.6 Hz, 1H), 7.40 (d, J = 7.6 Hz, 1H), 7.38-7.32 (m, |
| | 3H), 7.13-7.08 (m, 4H), 6.25 (d, J = 1.2 Hz, 1H), 2.45 (d, J = 1.2 Hz, 3H). |
| 136 | ¹H NMR (300 MHz, CDCl₃): δ 7.83 (d, J = 7.2 Hz, 2H), 7.76 (d, J = 9.0 Hz, 1H), |
| | 7.58 (d, J = 9.0 Hz, 1H), 7.56-7.53 (m, 3H), 7.48-7.37 (m, 4H), 6.28 (s, 1H), 2.52 (s, 3H). |
| 137 | ¹H NMR (300 MHz, CDCl₃): δ 7.82-7.78 (m, 2H), 7.75 (d, J = 9.3 Hz, 1H), 7.57 (d, J = 8.7 Hz, |
| | 1H), 7.54-7.49 (m, 1H), 7.45-7.42 (m, 2H), 7.38-7.30 (m, 4H), 6.27 (d, J = 1.2 Hz, |
| | 1H), 2.51 (d, J = 0.9 Hz, 3H). |
| 138 | ¹H NMR (300 MHz, CDCl₃): δ 7.81-7.74 (m, 3H), 7.59-7.44 (m, 5H), 7.38-7.33 (m, |
| | 2H), 7.28-7.22 (m, 1H), 6.27 (d, J = 1.2 Hz, 1H), 2.51 (d, J = 1.2 Hz, 3H). |
| | ¹³C NMR (75 MHz, CDCl₃): δ 185.1, 159.2, 156.3, 152.8, 149.7, 148.3, 136.4, 133.6, |
| | 133.1, 131.7, 129.5, 129.23, 129.15, 128.2, 127.1, 124.4, 121.6, 116.0, 115.4, 113.7, |
| | 108.9, 19.5. |
| 139 | ¹H NMR (300 MHz, CDCl₃): δ 7.81-7.74 (m, 3H), 7.60-7.44 (m, 5H), 7.38-7.33 (m, |
| | 2H), 7.28-7.22 (m, 1H), 6.23 (d, J = 1.2 Hz, 1H), 2.46 (d, J = 1.2 Hz, 3H). |
| | ¹³C NMR (75 MHz, CDCl₃): δ 185.1, 159.2, 156.3, 152.8, 149.7, 148.3, 136.4, 133.6, |
| | 133.1, 131.7, 129.5, 129.23, 129.15, 128.2, 127.1, 124.4, 121.6, 116.0, 115.4, 113.7, |
| | 108.9, 19.5. |
| 140 | ¹H NMR (300 MHz, CDCl₃): δ 7.81-7.73 (m, 3H), 7.58-7.45 (m, 4H), 7.39-7.26 (m, |
| | 4H), 6.27 (s, 1H), 2.50 (s, 3H). ¹³C NMR (75 MHz, CDCl₃): δ 185.2, 159.4, 156.3, |
| | 152.9, 152.8, 149.8, 148.2, 136.5, 133.6, 133.1, 132.2, 131.7, 131.0, 129.7, 129.5, |
| | 129.24, 129.17, 128.6, 128.2, 127.5, 124.4, 124.4, 123.2, 121.6. 116.0, 115.4, 113.7, |
| | 113.6, 109.0, 19.6. |
| 141 | ¹H NMR (300 MHz, CDCl₃): δ 7.76-7.72 (m, 3H), 7.57 (d, J = 9.0 Hz, 1H), |
| | 7.46-7.41 (m, 1H), 7.33-7.20 (m, 5H), 7.10 (d, J = 7.5 Hz, 1H), 6.25 (d, J = 0.9 Hz, 1H), 2.50 (d, J = 0.9 Hz, |
| | 3H), 2.25 (s, 3H). |
| 142 | ¹H NMR (300 MHz, CDCl₃): δ 7.78-7.68 (m, 3H), 7.53-7.45 (m, 2H), 7.42-7.27 (m, |
| | 4H), 7.13-7.10 (m, 2H), 6.22 (s, 1H), 2.46 (s, 3H), 2.32 (s, 3H). ¹³C NMR (75 MHz, |
| | CDCl₃): δ 185.4, 159.5, 156.3, 152.9, 149.8, 147.9, 138.4, 136.5, 132.7, 130.5, 129.6, |
| | 128.7, 128.4, 128.0, 127.8, 126.4, 124.1, 116.2, 115.2, 113.3, 108.8, 21.3, 19.4. |
| 143 | ¹H NMR (300 MHz, CDCl₃): δ 7.88-7.76 (m, 5H), 7.75-7.51 (m, 4H), 7.43-7.38 (m, |
| | 2H), 6.27 (d, J = 1.2 Hz, 1H), 2.51 (d, J = 1.2 Hz, 3H). ¹³C NMR (75 MHz, CDCl₃): δ |
| | 184.6, 159.0, 156.1, 152.7, 149.6, 148.5, 136.3, 134.8, 134.1, 133.3, 132.2, 131.3, |
| | 129.7, 128.7, 128.4, 126.3, 124.7, 118.4, 115.9, 115.6, 113.8, 112.1, 109.0, 19.5. |
| 144 | ¹H NMR (300 MHz, CDCl₃): δ 8.25-7.61 (m, 11H), 6.21 (s, 1H), 2.51 (d, J = 1.2 Hz, |
| | 3H). LCMS [M + 1]⁺: 426.0. |
| 145 | ¹H NMR (300 MHz, CDCl₃): δ 8.27-8.24 (m, 2H), 7.90-7.80 (m, 2H), 7.77-7.70 (m, |
| | 3H), 7.70-7.53 (m, 2H), 7.44-7.27 (m, 2H), 6.28 (d, J = 0.9 Hz, 1H), 2.52 (d, J = 0.9 Hz, |
| | 3H). ¹³C NMR (75 MHz, CDCl₃): δ 184.6, 159.1, 156.2, 152.8, 147.9, 136.9, 136.3, |
| | 133.5, 131.5, 130.2, 129.8, 129.5, 128.4, 128.2, 126.4, 124.7, 123.0, 115.7, 113.9, |
| | 109.1, 19.5. |
| 146 | ¹H NMR (300 MHz, CDCl₃): δ 7.75-7.30 (m, 11H), 6.21 (s, 1H), 3.82 (s, 2H), 2.33 (s, |
| | 3H). |
| 147 | ¹H NMR (300 MHz, CDCl₃): δ 7.89-7.21 (m, 14H), 6.22 (s, 1H), 2.47 (s, 3H). |
| 148 | ¹H NMR (400 MHz, CDCl₃): δ 7.85 (d, J = 7.2 Hz, 2H), 7.74 (d, J = 8.8 Hz, 1H), |
| | 7.59 (d, J = 8.7 Hz, 1H), 7.47 (t, J = 7.2 Hz, 1H), 7.40-7.33 (m, 4H), 7.13-7.12 (m, 2H), |
| | 6.25 (d, J = 1.2 Hz, 1H), 2.50 (s, 3H). |
| 149 | ¹H NMR (300 MHz, CDCl₃): δ 7.85 (dd, J = 8.7, 1.8 Hz, 2H), 7.58 (d, J = 8.7 Hz, 2H), |
| | 7.62 (d, J = 8.7 Hz, 1H), 7.54 (d, J = 7.5 Hz, 1H), 7.43 (d, J = 7.5 Hz, 1H), 7.39 (d, J = 1.8 Hz, |
| | 2H), 7.35 (dd, J = 1.8, 1.8 Hz, 1H), 6.28 (d, J = 1.2 Hz, 1H), 2.51 (d, J = 1.2 Hz, |
| | 3H). |
| 150 | ¹H NMR (300 MHz, CDCl₃): δ 7.84 (d, J = 6.9 Hz, 2H), 7.76 (d, J = 9.0 Hz, 1H), |
| | 7.64 (t, J = 1.8 Hz, 1H), 7.59-7.53 (m, 4H), 7.43 (t, J = 6.0 Hz, 2H), 6.29 (d, J = 1.5 Hz, 1H), |
| | 2.52 (d, J = 1.5 Hz, 3H). LCMS [M + 1]⁺: 539.9. |
| 151 | ¹H NMR (300 MHz, CDCl₃): δ 9.13 (t, J = 2.1 Hz, 1H), 8.81 (t, J = 2.1 Hz, 2H), |
| | 7.92 (dd, J = 8.4, 1.5 Hz, 2H), 7.83 (d, J = 9.0 Hz, 1H), 7.65-7.61 (m, 2H), 7.50 (t, J = 7.2 Hz, |
| | 2H), 6.28 (d, J = 0.9 Hz, 1H), 2.51 (d, J = 0.9 Hz, 3H). |
| 153 | ¹H NMR (300 MHz, CDCl₃): δ 8.05-8.01 (m, 2H), 7.83 (s, 1H), 7.83-7.68 (m, 2H), |
| | 7.66-7.28 (m, 3H), 6.32 (d, J = 0.9 Hz, 1H), 2.53 (s, 3H). ¹³C NMR (75 MHz, CDCl₃): |
| | 183.7, 160.1, 157.6, 153.3, 152.6, 149.0, 136.6, 133.3, 129.3, 128.7, 124.5, 117.0, |
| | 115.1, 113.6, 113.4, 109.1, 22.6. |
| 154 | ¹H NMR (300 MHz, CDCl₃): δ 8.06-8.03 (m, 2H), 7.67 (d, J = 8.7 Hz, 1H), |
| | 7.64-7.60 (m, 1H), 7.56-7.51 (m, 3H), 7.45 (d, J = 8.7 Hz, 1H), 6.29 (d, J = 1.2 Hz, 1H), 2.91 (s, |
| | 3H), 2.51 (d, J = 0.9 Hz, 3H). |
| 155 | ¹H NMR (300 MHz, CDCl₃): δ 8.06-7.44 (m, 7H), 6.32 (d, J = 1.2 Hz, 1H), 3.37 (t, J = 5.4 Hz, |
| | 2H), 2.52 (d, J = 1.2 Hz, 3H), 2.52 (d, J = 1.2 Hz, 3H), 1.87 (sex, J = 7.2 Hz, |
| | 2H), 1.07 (t, J = 7.2 Hz, 3H). |
| 156 | ¹H NMR (300 MHz, CDCl₃): δ 8.07-8.03 (m, 2H), 7.69-7.43 (m, 5H), 6.30 (s, 1H), |
| | 2.57 (d, J = 1.2 Hz, 3H), 1.83-1.34 (m, 8H), 0.89 (t, J = 7.2 Hz, 3H). ¹³C NMR (75 MHz, |
| | CDCl₃): δ 185.4, 159.9, 159.8, 156.2, 153.1, 153.0, 150.2, 148.5, 148.3, 137.6, 132.8, |
| | 132.2, 131.8, 129.6, 128.6, 128.3, 128.1, 124.0, 117.43, 117.35, 114.9, 113.2, 108.8, |
| | 39.3. 31.6, 29.9, 28.4, 25.1, 23.3, 22.4, 19.5, 14.0. |
| 157 | ¹H NMR (300 MHz, CDCl₃): δ 8.06-7.52 (m, 7H), 6.31 (d, J = 1.2 Hz, 1H), 3.38 (t, J = 7.5 Hz, |
| | 2H), 2.52 (d, J = 1.2 Hz, 3H), 1.80 (quin, J = 7.8 Hz, 2H), 1.62-1.26 (m, 6H), |
| | 0.87 (t, J = 6.9 Hz, 3H). |
| 158 | ¹H NMR (300 MHz, CDCl₃): δ 8.06-7.44 (m, 7H), 6.31 (s, 1H), 3.38 (t, J = 7.5 Hz, |
| | 2H), 2.51 (s, 3H), 1.80 (quin, J = 7.5 Hz, 2H), 1.63 (bs, 2H), 1.48 (quin, J = 7.2 Hz, |
| | 2H), 1.43 (bs, 10H), 0.86 (t, J = 6.3 Hz, 3H). |
| 159 | ¹H NMR (300 MHz, CDCl₃): δ 7.84-7.24 (m, 9H), 6.48-6.46 (m, 1H), 6.28 (s, 1H), |
| | 2.49 (s, 3H). ¹³C NMR (75 MHz, CDCl₃): δ 185.5, 159.6, 156.4, 153.0, 149.3, 148.0, 143.3, |
| | 142.8, 136.8, 133.0, 129.2, 128.3, 124.1, 116.9, 115.4, 114.6, 114.4, 113.3, 111.9, |
| | 108.9, 19.5. |
| 160 | ¹H NMR (300 MHz, CDCl₃): δ 7.83-7.29 (m, 10H), 6.26 (d, J = 0.6 Hz, 1H), 2.48 (d, J = 0.6 Hz, |
| | 3H). ¹³C NMR (75 MHz, CDCl₃): δ 185.2, 159.5, 156.2, 152.9, 149.7, 148.5, |
| | 136.4, 133.1, 131.3, 129.5, 129.2, 128.3, 128.2, 126.8, 124.3, 120.9, 116.0, 115.4, |
| | 113.5, 108.9, 19.5. |
| 161 | ¹H NMR (300 MHz, CDCl₃): δ 8.17-7.95 (m, 4H), 7.77-7.30 (m, 10H), 6.37 (s, 1H), |
| | 2.55 (s, 3H). |
| 165 | ¹H NMR (400 MHz, CDCl₃): δ 7.95-7.93 (m, 2H), 7.67 (d, J = 8.8 Hz, 1H), |
| | 7.64-7.60 (m, 1H), 7.53-7.49 (m, 2H), 7.43 (d, J = 8.8 Hz, 1H), 6.30 (q, J = 1.2 Hz, 1H), 3.68 (tt, J = 3.6, |
| | 12.4 Hz, 1H), 2.50 (d, J = 1.2 Hz, 3H), 2.34-2.23 (m, 2H), 1.89-1.85 (m, 2H), |
| | 1.74-1.71 (m, 3H), 1.65-1.42 (m, 1H), 1.42-1.33 (m, 2H). ¹³C NMR (100 MHz, |
| | CDCl₃): δ 186.5 (C), 159.8 (C), 156.8 (C), 153.1 (C), 149.4 (C), 148.1 (C), 137.9 (C), |
| | 135.0 (C), 133.1 (CH), 129.7 (CH × 2), 128.4 (CH × 2), 123.8 (CH), 117.1 (C), 115.1 (C), |
| | 113.0 (CH), 108.9 (CH), 35.1 (CH), 30.6 (CH₂ × 2), 26.7 (CH₂ × 2), 25.5 (CH₂), |
| | 19.7 (CH₃). EIMS m/z (relative intensity) 386 (M⁺, 54), 329 (24), 317 (100), 203 (28), |
| | 105 (55), 78 (62), 63 (90), 57 (63). HRMS Calcd. for C₂₅H₁₂O₄ 386.1518, found 386.1518. |
| | IR (neat): 3058, 2923, 2852, 1738, 1636, 1599, 1538, 1468, 1447 cm⁻¹. |
| 166 | ¹H NMR (600 MHz, CDCl₃): δ 7.75 (d, J = 8.0 Hz, 1H), 7.72 (d, J = 8.8 Hz, 1H), |
| | 7.56 (d, J = 8.8 Hz, 1H), 7.46-7.39 (m, 3H), 7.34-7.14 (m, 6H), 6.23 (s, 1H), 2.48 (s, 3H). |
| | ¹³C NMR (100 MHz, CDCl₃): δ 220.0 (C), 159.4 (C), 157.4 (C), 154.8 (C), 152.8 (C), |
| | 150.4 (C), 145.4 (C), 131.8 (CH), 130.8 (CH × 2), 130.3 (C), 129.0 (CH × 2), 128.3 (CH), |
| | 127.6 (CH × 3), 124.7 (C), 124.3 (CH), 117.2 (C), 115.4 (C), 113.4 (CH), 108.8 (CH), |
| | 19.5 (CH₃). |
| | EIMS m/z (relative intensity) 396 (M⁺, 60), 378 (100), 367 (26), 189 (10), 105 (32), |
| | 83 (75), 77 (20). HRMS Calcd. for C₂₅H₁₆O₃S 396.082, found 396.0823. IR (neat): 2918, |
| | 2851, 1735, 1647, 1600, 1541, 1488, 1443, 1381, 1356, 1155, 1078 cm⁻¹. |
| 170 | ¹H NMR (300 MHz, CDCl₃): δ 7.60-7.55 (m, 2H), 7.52-7.44 (m, 6H), 7.43-7.29 (m, |
| | 4H), 6.18 (d, J = 1.2 Hz, 1H), 5.97 (s, 1H), 2.76 (s, 1H), 2.44 (d, J = 0.9 Hz, 3H). |
| 171 | LCMS [M + 1]⁺: 396.1 |
| 172 | ¹H NMR (300 MHz, CDCl₃): δ 7.58-7.54 (m, 2H), 7.52-7.44 (m, 6H), 7.42-7.28 (m, |
| | 4H), 6.18 (d, J = 1.2 Hz, 1H), 5.96 (d, J = 5.4 Hz, 1H), 2.76 (d, J = 5.4 Hz, 1H), 2.44 (d, |
| | J = 1.2 Hz, 3H). LCMS [M + 1]⁺: 383.0. |
| 173 | ¹H NMR (300 MHz, CDCl₃): δ 7.57-7.23 (m, 12H), 6.19 (d, J = 0.9 Hz, 1H), 4.15 (s, |
| | 2H), 2.25 (d, J = 0.9 Hz, 3H). LCMS [M + 1]⁺: 367.1. |
| 174 | LCMS [M + 1]⁺: 438.1 |
| 175 | LCMS [M + 1]⁺: 486.1 |
| 176 | LCMS [M + 1]⁺: 410.1 |
| 177 | ¹H NMR (300 MHz, CD₃OD): δ 7.82 (d, J = 8.7 Hz, 1H), 7.55 (d, J = 8.7 Hz, 1H), |
| | 7.41-7.01 (m, 9H), 6.21 (d, J = 1.2 Hz, 1H), 4.06-3.54 (m, 12H), 2.47 (d, J = 0.9 Hz, |
| | 3H). |
| 178 | ¹H NMR (300 MHz, CD₃OD): δ 7.91 (d, J = 8.7 Hz, 1H), 7.63 (d, J = 9.0 Hz, 1H), |
| | 7.46-7.03 (m, 9H), 6.27 (d, J = 1.2 Hz, 1H), 4.09-3.58 (m, 16H), 2.52 (d, J = 1.2 Hz, |
| | 3H). |
| 179 | ¹H NMR (400 MHz, CDCl₃): δ 7.74 (d, J = 8.8 Hz, 1H), 7.58 (d, J = 8.8 Hz, 1H), |
| | 7.49-7.32 (m, 7H), 7.19 (t, J = 8.0 Hz, 1H), 7.00 (dd, J = 8.0, 2.4 Hz, 1H), 6.26 (d, J = 1.2 Hz, |
| | 1H), 3.74 (d, J = 5.8 Hz, 2H), 2.51 (d, J = 1.2 Hz, 3H), 1.25 (bs, 1H), 0.65 (q, J = 5.8 Hz, |
| | 2H), 0.34 (q, J = 4.8 Hz, 2H). |
| 180 | ¹H NMR (300 MHz, CDCl₃): δ 7.75 (d, J = 8.7 Hz, 1H), 7.64 (dt, J = 8.1, 1.2 Hz, 1H), |
| | 7.58 (d, J = 8.7 Hz, 1H), 7.56-7.18 (m, 8H), 6.26 (d, J = 1.2 Hz, 1H), 2.51 (d, J = 1.2 Hz, |
| | 3H), 2.30 (s, 3H). |
| 181 | ¹H NMR (300 MHz, CDCl₃): δ 7.75 (d, J = 9.0 Hz, 1H), 7.59 (d, J = 9.0 Hz, 1H), |
| | 7.63-7.19 (m, 9H), 6.26 (s, 1H), 2.51 (s, 3H), 1.83-1.79 (m, 1H), 1.19-1.10 (m, 2H), |
| | 1.09-1.00 (m, 2H). |
| 182 | ¹H NMR (300 MHz, CDCl₃): δ 7.75 (d, J = 9.0 Hz, 1H), 7.64 (dt, J = 7.8, 1.5 Hz, 1H), |
| | 7.60-7.23 (m, 9H), 6.66 (dd, J = 17.4, 1.5 Hz, 1H), 6.26 (d, J = 1.2 Hz, 1H), 6.04 (dd, J = 10.5, |
| | 1.2 Hz, 1H), 2.51 (d, J = 1.2 Hz, 3H). |
| 183 | ¹H NMR (300 MHz, CDCl₃): δ 7.73 (d, J = 9.0 Hz, 1H), 7.58 (d, J = 9.0 Hz, 1H), |
| | 7.45-7.29 (m, 7H), 7.00 (ddd, J = 8.4, 2.4, 0.9 Hz, 1H), 6.25 (d, J = 1.2 Hz, 1H), 4.08 (t, J = 5.4 Hz, |
| | 2H), 3.03 (t, J = 5.4 Hz, 2H), 2.50 (d, J = 1.2 Hz, 3H), 2.02-1.25 (m, 14H). |
| 184 | ¹H NMR (300 MHz, CDCl₃): δ 7.74 (d, J = 9.0 Hz, 1H), 7.58 (d, J = 8.7 Hz, 1H), |
| | 7.49-7.00 (m, 9H), 6.26 (s, 1H), 4.04 (t, J = 4.5 Hz, 2H), 3.94 (t, J = 3.9 Hz, 2H), 2.50 (s, |
| | 3H). |
| 185 | ¹H NMR (300 MHz, CDCl₃): δ 7.73 (d, J = 8.7 Hz, 1H), 7.58 (d, J = 8.7 Hz, 1H), |
| | 7.49-6.97 (m, 9H), 6.25 (d, J = 1.2 Hz, 1H), 4.06 (t, J = 6.0 Hz, 2H), 3.85 (t, J = 6.0 Hz, 2H), |
| | 2.50 (d, J = 1.2 Hz, 3H), 2.06-1.98 (m, 2H). |
| 186 | ¹H NMR (300 MHz, CDCl₃): δ 7.73 (d, J = 9.0 Hz, 1H), 7.58 (d, J = 9.0 Hz, 1H), |
| | 7.50-6.96 (m, 9H), 6.25 (d, J = 0.9 Hz, 1H), 3.93 (t, J = 6.0 Hz, 2H), 3.47 (m, 2H), 2.50 (s, |
| | 3H), 2.10-1.64 (m, 4H). |
| 187 | ¹H NMR (300 MHz, CDCl₃): δ 7.74 (d, J = 9.0 Hz, 1H), 7.58 (d, J = 9.0 Hz, 1H), |
| | 7.51-6.96 (m, 9H), 6.26 (d, J = 1.2 Hz, 1H), 3.91 (t, J = 6.3 Hz, 2H), 3.44 (t, J = 6.3 Hz, 2H), |
| | 2.51 (d, J = 0.6 Hz, 3H), 1.98-1.56 (m, 6H). |
| 188 | ¹H NMR (300 MHz, CD₃OD): δ 7.88 (d, J = 9.0 Hz, 1H), 7.60 (d, J = 9.0 Hz, 1H), |
| | 7.44-6.98 (m, 9H), 6.25 (d, J = 1.2 Hz, 1H), 3.89 (t, J = 6.3 Hz, 2H), 3.56 (t, J = 6.6 Hz, |
| | 2H), 2.50 (d, J = 1.2 Hz, 3H), 1.78-1.42 (m, 8H). |
| 189 | ¹H NMR (400 MHz, CDCl₃): δ 7.73 (d, J = 6.6 Hz, 1H), 7.57 (d, J = 6.6 Hz, 1H), |
| | 7.49-6.99 (m, 9H), 6.25 (d, J = 0.9 Hz, 1H), 3.99-3.93 (m, 4H), 2.50 (d, J = 0.9 Hz, 3H), |
| | 0.90 (s, 9H), 0.09 (s, 6H). |
| 190 | ¹H NMR (300 MHz, CDCl₃): δ 7.74 (d, J = 9.0 Hz, 1H), 7.58 (d, J = 9.0 Hz, 1H), |
| | 7.50-6.97 (m, 9H), 6.26 (d, J = 1.2 Hz, 1H), 4.02 (t, J = 6.3 Hz, 2H), 3.78 (t, J = 6.0 Hz, 2H), |
| | 2.51 (d, J = 1.2 Hz, 3H), 1.96 (t, J = 6.0 Hz, 2H), 0.88 (s, 9H), 0.042 (s, 6H). |
| 191 | ¹H NMR (300 MHz, CDCl₃): δ 7.73 (d, J = 9.0 Hz, 1H), 7.57 (d, J = 9.0 Hz, 1H), |
| | 7.50-6.96 (m, 9H), 6.25 (d, J = 1.2 Hz, 1H), 3.93 (t, J = 6.3 Hz, 2H), 3.68 (t, J = 6.3 Hz, 2H), |
| | 2.50 (d, J = 0.9 Hz, 3H), 1.85-1.65 (m, 4H), 0.90 (s, 9H), 0.05 (s, 6H). |
| 193 | ¹H NMR (300 MHz, CDCl₃): δ 7.74 (d, J = 9.0 Hz, 1H), 7.58 (d, J = 9.0 Hz, 1H), |
| | 7.50-6.96 (m, 9H), 6.26 (d, J = 1.2 Hz, 1H), 3.90 (t, J = 6.3 Hz, 2H), 3.62 (t, J = 6.6 Hz, 2H), |
| | 2.51 (d, J = 1.2 Hz, 3H), 1.79-1.39 (m, 8H), 0.89 (s, 9H), 0.05 (s, 6H). |
| 194 | LCMS [M + 1]⁺: 771.2. |
| 195 | ¹H NMR (400 MHz, CDCl₃): δ 7.93 (brs, 2H), 7.64 (brs, 1H), 7.44 (brs, 2H), 7.28 (brs, |
| | 5H), 7.05 (brs, 1H), 6.26 (brs, 1H), 4.42-3.45 (m, 11H), 2.53 (brs, 3H). |
| 196 | ¹H NMR (300 MHz, CDCl₃): δ 7.72 (d, J = 8.7 Hz, 1H), 7.57-7.54 (m, 1H), |
| | 7.48-7.45 (m, 2H), 7.36-7.31 (m, 5H), 7.19-7.13 (m, 1H), 6.99-6.95 (m, 1H), 6.22 (s, 1H), |
| | 5.42-5.30 (m, 2H), 5.21-4.94 (m, 3H), 4.47-4.43 (m, 1H), 4.12-3.43 (m, 7H), 2.16 (s, 3H). |
| 197 | ¹H NMR (300 MHz, CDCl₃): δ 7.95 (d, J = 9.0 Hz, 1H), 7.67 (d, J = 9.0 Hz, 1H), |
| | 7.47-7.45 (m, 2H), 7.33-7.19 (m, 6H), 7.05-7.03 (m, 1H), 6.29 (s, 1H), 4.26-4.22 (m, 1H), |
| | 4.09-3.46 (m, 16H), 2.55 (s, 3H). |
| 198 | ¹H NMR (300 MHz, CDCl₃): δ 7.86 (d, J = 15.9 Hz, 1H), 7.73 (d, J = 9.0 Hz, 1H), |
| | 7.67-7.30 (m, 15H), 6.61 (d, J = 15.9 Hz, 1H), 6.24 (d, J = 1.2 Hz, 1H), 2.48 (d, J = 1.2 Hz, |
| | 3H). |
| 199 | ¹H NMR (300 MHz, CDCl₃): δ 7.76 (d, J = 9.0 Hz, 1H), 7.62 (d, J = 9.0 Hz, 1H), |
| | 7.50-7.21 (m, 8H), 7.11 (dd, J = 8.4, 2.1 Hz, 1H), 6.26 (s, 1H), 4.47 (t, J = 4.2 Hz, 2H), |
| | 3.43 (t, J = 4.2 Hz, 2H), 2.91 (s, 6H), 2.51 (s, 3H). |
| 200 | ¹H NMR (300 MHz, CDCl₃): δ 7.73 (d, J = 9.0 Hz, 1H), 7.57 (d, J = 9.0 Hz, 1H), |
| | 7.49-6.96 (m, 14H), 6.25 (d, J = 1.2 Hz, 1H), 4.03 (t, J = 6.0 Hz, 2H), 2.95 (m, 2H), 2.74 (t, J = 6.0 Hz, |
| | 2H), 2.54-2.50 (m, 5H), 2.07-1.99 (m, 3H), 1.67-1.63 (m, 2H), 1.40-1.26 (m, |
| | 2H). |
| 201 | ¹H NMR (300 MHz, CDCl₃): δ 7.78-7.32 (m, 16H), 6.26 (d, J = 1.2 Hz, 1H), 2.39 (d, J = 1.2 Hz, |
| | 3H). |
| 202 | ¹H NMR (300 MHz, CDCl₃): δ 7.75 (d, J = 8.7 Hz, 1H), 7.66-7.19 (m, 10H), 6.26 (d, J = 1.2 Hz, |
| | 1H), 2.63 (dt, J = 7.2, 2.7 Hz, 2H), 2.59 (d, J = 1.2 Hz, 3H), 2.05~2.04 (m, |
| | 1H), 2.81 (t, J = 7.2 Hz, 2H). |
| 203 | ¹H NMR (300 MHz, CDCl₃): δ 7.75 (d, J = 8.7 Hz, 1H), 7.70-7.23 (m, 10H), 6.25 (s, |
| | 1H), 2.50 (s, 3H), 2.41 (t, J = 6.9 Hz, 2H), 1.64 (quin, J = 7.8 Hz, 2H), 1.47-1.29 (m, |
| | 4H), 0.93 (t, J = 6.9 Hz, 3H). |
| 204 | ¹H NMR (300 MHz, CDCl₃): δ 7.80 (dd, J = 8.7, 1.5 Hz, 1H), 7.77 (d, J = 8.7 Hz, 1H), |
| | 7.59 (d, J = 8.7 Hz, 1H), 7.56-7.28 (m, 6H), 7.00 (dd, J = 8.7, 1.2 Hz, 1H), 6.75 (td, J = 8.1, |
| | 0.9 Hz, 1H), 6.27 (d, J = 1.2 Hz, 1H), 2.52 (d, J = 1.2 Hz, 3H). LCMS [M + 1]⁺: |
| | 397.1. |
| 205 | ¹H NMR (300 MHz, CDCl₃): δ 7.74 (d, J = 9.0 Hz, 1H), 7.58-7.51 (m, 6H), 6.24 (s, |
| | 1H), 2.49 (s, 3H). |
| 206 | ¹H NMR (400 MHz, CDCl₃): δ 7.84-7.73 (m, 3H), 7.65-7.62 (m, 4H), 7.46-7.25 (m, |
| | 5H), 7.07-7.03 (m, 2H), 6.11 (d, J = 0.9 Hz, 1H), 2.44 (d, J = 0.6 Hz, 3H). |
| 207 | LCMS [M + 1]⁺: 425.1. |
| 208 | ¹H NMR (300 MHz, CDCl₃): δ 8.34 (s, 1H), 7.88-7.82 (m, 4H), 7.79-7.71 (m, 1H), |
| | 7.62-7.49 (m, 3H), 7.40 (s, 4H), 6.25 (d, J = 0.9 Hz, 1H), 2.48 (s, 3H). |
| | ¹³C NMR (75 MHz, CDCl₃): δ 184.9, 159.3, 156.3, 152.9, 149.7, 148.4, 135.4, 133.6, |
| | 132.1, 132.0, 131.0, 129.4, 128.74, 128.68, 128.2, 127.8, 127.4, 126.8, 124.8, 124.3, |
| | 123.1, 115.9, 115.4, 113.5, 109.0, 19.5. |
| 209 | ¹H NMR (300 MHz, CDCl₃): δ 7.91-7.86 (m, 2H), 7.75 (d, J = 8.7 Hz, 1H), |
| | 7.58-7.49 (m, 3H), 7.41-7.38 (m, 2H), 7.27-7.03 (m, 2H), 6.27 (s, 1H), 2.51 (s, 3H). |
| | ¹³C NMR (75 MHz, CDCl₃): δ 183.4, 167.4, 164.0, 159.3, 156.2, 152.8, 149.8, 147.9, |
| | 132.8, 132.7, 132.5, 132.4, 132.1, 131.0, 128.5, 127.6, 124.5, 123.3, 115.9, 115.7, |
| | 115.5, 115.4, 113.7, 108.9, 19.6. |
| 210 | ¹H NMR (300 MHz, CDCl₃): δ 7.80-7.74 (m, 3H), 7.57-7.50 (m, 3H), 7.40-7.26 (m, |
| | 4H), 6.27 (s, 1H), 2.50 (s, 3H). ¹³C NMR (75 MHz, CDCl₃): δ 183.6, 159.3, 156.2, |
| | 152.8, 149.8, 147.8, 139.7, 134.8, 132.1, 131.1, 128.6, 128.4, 127.9, 124.6, 123.4, |
| | 115.9, 115.5, 113.7, 108.9, 19.5. |
| 211 | ¹H NMR (300 MHz, CDCl₃): δ 7.77-7.69 (m, 4H), 7.58-7.51 (m, 6H), 7.40-7.37 (m, |
| | 2H), 6.27 (d, J = 0.9 Hz, 1H), 2.510 (d, J = 0.9 Hz, 3H). ¹³C NMR (75 MHz, CDCl₃): |
| | δ 183.8, 159.3, 156.3, 152.8, 149.8, 147.8, 135.3, 132.1, 131.6, 131.2, 131.1, 128.4, |
| | 128.0, 124.6, 123.5, 116.0, 115.5, 113.7, 109.0, 19.6. |
| 212 | ¹H NMR (300 MHz, CDCl₃): δ 7.74-7.70 (m, 3H), 7.55 (dd, J = 9.0, 0.9 Hz, 1H), |
| | 7.48 (dd, J = 6.0, 2.1 Hz, 2H), 7.38 (dd, J = 6.0, 2.1 Hz, 2H), 7.16 (d, J = 8.7 Hz, 2H), |
| | 6.26 (d, J = 0.9 Hz, 1H), 2.50 (s, 3H), 2.382 (s, 3H). ¹³C NMR (75 MHz, CDCl₃): δ 184.8, |
| | 159.4, 156.3, 152.9, 149.7, 148.4, 144.3, 138.8, 132.2, 131.0, 129.9, 129.0, 128.7, |
| | 127.0, 124.2, 123.1, 116.0, 115.4, 113.6, 109.0, 21.7, 19.6. |
| 213 | ¹H NMR (300 MHz, CDCl₃): δ 7.89 (d, J = 8.1 Hz, 2H), 7.78 (d, J = 9.0 Hz, 1H), |
| | 7.64-7.55 (m, 3H), 7.50-7.47 (m, 2H), 7.38-7.27 (m, 2H), 2.51 (s, 3H). |
| | ¹³C NMR (75 MHz, CDCl₃): δ 183.9, 159.2, 156.4, 152.8, 149.9, 147.5, 139.6, 134.3, |
| | 133.9, 132.1, 131.0, 129.8, 128.8, 128.2, 125.20, 125.15, 125.11, 123.6, 115.9, 115.6, |
| | 113.8, 109.0, 19.5. |
| 214 | ¹H NMR (300 MHz, CDCl₃): δ 7.85-7.82 (m, 2H), 7.72 (d, J = 8.7 Hz, 1H), |
| | 7.57-7.47 (m, 3H), 7.41-7.38 (m, 2H), 6.85-6.83 (m, 2H), 6.25 (s, 1H), 3.85 (s, 3H), 2.50 (s, 3H). |
| | ¹³C NMR (75 MHz, CDCl₃): δ 183.5, 163.8, 159.4, 156.2, 152.9, 149.6, 148.6, 132.3, |
| | 132.2, 131.0, 129.0, 128.8, 126.5, 124.0, 123.0, 115.9, 115.4, 113.6, 113.5, 108.9, 55.5, |
| | 19.5. |
| 215 | ¹H NMR (300 MHz, CDCl₃): δ 7.89-7.86 (m, 2H), 7.74 (d, J = 9.0 Hz, 1H), |
| | 7.61-7.55 (m, 5H), 7.50-7.36 (m, 7H), 2.50 (d, J = 0.9 Hz, 3H). ¹³C NMR (75 MHz, CDCl₃): |
| | δ 184.6, 159.4, 156.3, 152.9, 149.8, 148.3, 146.0, 139.7, 135.1, 132.2, 131.0, 130.3, |
| | 128.9, 128.7, 128.3, 127.5, 127.3, 126.9, 124.4, 123.2, 116.0, 115.4, 113.6, 109.0, 19.6. |
| 216 | ¹H NMR (300 MHz, CDCl₃): δ 8.25-8.22 (m, 2H), 8.00-7.97 (m, 2H), 7.80 (d, J = 8.7 Hz, |
| | 1H), 7.59-7.51 (m, 3H), 7.42-7.39 (m, 2H), 6.29 (d, J = 1.2 Hz, 1H), 2.52 (d, J = 1.2 Hz, |
| | 3H). |
| | ¹³C NMR (75 MHz, CDCl₃): δ 183.0, 159.1, 156.4, 152.7, 145.0, 149.9, 147.2, 141.6, |
| | 132.1, 131.2, 130.5, 129.4, 128.1, 125.3, 123.8, 123.4, 116.0, 115.7, 113.9, 109.0, 19.6. |
| 217 | ¹H NMR (300 MHz, CDCl₃): δ 7.78-7.66 (m, 3H), 7.57 (d, J = 9.0 Hz, 1H), |
| | 7.50-7.46 (m, 3H), 7.38-7.27 (m, 3H), 6.27 (d, J = 1.2 Hz, 1H), 2.51 (d, J = 1.2 Hz, 3H). |
| | ¹³C NMR (75 MHz, CDCl₃): δ 183.6, 159.2, 156.4, 152.8, 149.8, 147.6, 138.0, 134.4, |
| | 132.8, 132.1, 131.0, 129.64, 129.58, 128.4, 128.3, 127.6, 124.8, 123.441, 115.9, 115.5, |
| | 113.7, 109.0, 19.5. |
| 218 | ¹H NMR (300 MHz, CDCl₃): δ 7.90 (d, J = 2.1 Hz, 1H), 7.78 (d, J = 9.0 Hz, 1H), |
| | 7.69 (dd, J = 9.0, 2.1 Hz, 1H), 7.60-7.46 (m, 4H), 7.41-7.37 (m, 2H), 6.28 (d, J = 1.2 Hz, |
| | 1H), 2.52 (d, J = 1.2 Hz, 3H). ¹³C NMR (75 MHz, CDCl₃): δ 182.4, 159.2, 156.4, |
| | 152.8, 149.9, 147.4, 137.8, 136.0, 132.9, 132.1, 131.6, 131.2, 130.4, 129.5, 128.6, |
| | 128.5, 128.3, 125.0, 123.7, 116.0, 115.6, 113.8, 109.0, 19.6. |
| 219 | ¹H NMR (300 MHz, CDCl₃): δ 7.71 (d, J = 9.0 Hz, 1H), 7.54 (d, J = 9.0 Hz, 1H), |
| | 7.38-7.30 (m, 3H), 7.22-7.19 (m, 2H), 6.93-6.89 (m, 1H), 6.58 (d, J = 9.0 Hz, 1H), 6.23 (d, J = 1.2 Hz, |
| | 1H), 3.63 (s, 3H), 2.48 (d, J = 1.2 Hz, 3H). ¹³C NMR (75 MHz, CDCl₃): |
| | δ 185.2, 159.4, 157.3, 156.3, 152.9, 149.9, 149.2, 133.0, 131.9, 130.3, 130.0, 128.3, |
| | 127.7, 127.0, 124.4, 122.9, 120.4, 116.2, 115.2, 113.4, 110.6, 109.0, 55.3, 19.5. |
| 220 | ¹H NMR (300 MHz, CDCl₃): δ 7.89-7.38 (m, 11H), 6.89-6.83 (m, 2H), 6.14 (d, J = 1.2 Hz, |
| | 1H), 2.38 (d, J = 0.9 Hz, 3H). |
| 221 | ¹H NMR (300 MHz, CDCl₃): δ 7.68 (d, J = 9.0 Hz, 1H), 7.47 (d, J = 9.0 Hz, 1H), |
| | 7.39-7.32 (m, 2H), 7.19-7.08 (m, 1H), 7.04-6.89 (m, 3H), 6.81-6.72 (m, 1H), 6.17 (d, J = 1.2 Hz, |
| | 1H), 2.41 (d, J = 0.9 Hz, 1H). |
| 222 | ¹H NMR (300 MHz, CDCl₃): δ 7.70-7.48 (m, 4H), 7.42-7.35 (m, 4H), 6.18 (d, J = 1.2 Hz, |
| | 1H), 2.31 (d, J = 1.2 Hz, 3H). |
| 223 | ¹H NMR (300 MHz, CDCl₃): δ 7.87-7.82 (m, 2H), 7.73 (d, J = 8.7 Hz, 1H), 7.563 (d, J = 8.7 Hz, |
| | 1H), 7.48-7.44 (m, 2H), 7.36-7.32 (m, 2H), 6.87-6.82 (m, 2H), 6.27 (d, J = 1.2 Hz, |
| | 1H), 3.85 (s, 3H), 2.51 (d, J = 0.9 Hz, 3H). |
| 224 | ¹H NMR (300 MHz, CDCl₃): δ 7.80 (d, J = 7.2 Hz, 2H), 7.75 (d, J = 8.7 Hz, 1H), |
| | 7.58 (d, J = 8.7 Hz, 1H), 7.53-7.48 (m, 2H), 7.44-7.30 (m, 4H), 6.27 (s, 1H), 2.51 (s, 3H). |
| 225 | ¹H NMR (300 MHz, CDCl₃): δ 7.81-7.77 (m, 2H), 7.46-7.35 (m, 5H), 6.19 (d, J = 0.9 Hz, |
| | 1H), 2.42 (d, J = 1.2 Hz, 3H). ¹³C NMR (75 MHz, CDCl₃): δ 160.7, 156.8, 156.7, |
| | 153.5, 129.3, 129.0, 128.8, 124.9, 120.2, 118.5, 114.6, 114.5, 112.6, 107.9, 98.3, 19.2. |
| 226 | ¹H NMR (300 MHz, CDCl₃): δ 7.50-7.19 (m, 7H), 6.81 (s, 1H), 6.16 (d, J = 0.9 Hz, |
| | 1H), 5.93 (s, 1H), 3.70 (brs, 1H), 2.40 (d, J = 0.9 Hz, 3H). |
| 227 | ¹H NMR (300 MHz, CDCl₃): δ 8.16 (dd, J = 8.1, 1.5 Hz, 2H), 7.58 (d, J = 8.7 Hz, 1H), |
| | 7.51-6.45 (m, 4H), 2.68 (s, 3H). ¹³C NMR (75 MHz, CDCl₃): δ 156.1, 155.2, 151.9, |
| | 151.5, 146.2, 129.7, 129.6, 128.7, 128.6, 127.0, 121.8, 117.2, 115.1, 111.4, 108.8, 90.5, |
| | 20.3. |
| 228 | ¹H NMR (400 MHz, CDCl₃): δ 7.50-7.48 (m, 1H), 7.42-7.38 (m, 3H), 7.34-7.20 (m, |
| | 6H), 7.06 (d, J = 7.2 Hz, 2H), 6.12 (q, J = 1.2 Hz, 1H), 4.19 (s, 2H), 3.56 (s, 3H), |
| | 2.46 (d, J = 1.2 Hz, 3H). ¹³C NMR (100 MHz, CDCl₃): δ 161.0 (C), 153.7 (C), 148.8 (C), |
| | 139.3 (C), 138.5 (C), 136.2 (C), 134.3 (C), 130.6 (CH × 2), 128.7 (CH × 2), 127.82 (CH × 2), |
| | 127.79 (CH × 2), 126.9 (CH), 126.5 (CH), 117.3 (CH), 116.8 (C), 114.5 (C), |
| | 111.9 (C), 111.3 (CH), 105.9 (CH), 30.7 (CH₂), 30.6 (CH₃), 19.6 (CH₃). EIMS m/z (relative |
| | intensity) 379 (M⁺, 100), 351 (6), 302 (13), 274 (8), 150 (5), 84 (11). HRMS Calcd. for |
| | C₂₆H₂₁NO₂ 379.4504, found 379.1559. |
| 229 | ¹H NMR (300 MHz, d6-DMSO): δ 11.78 (br, 1H), 7.46-7.11 (m, 12H), 6.14 (q, J = 1.2 Hz, |
| | 1H), 4.07 (s, 2H), 2.45 (d, J = 1.2 Hz, 3H). ¹³C NMR (75 MHz, d6-DMSO): |
| | δ 160.0 (C), 154.8 (C), 148.2 (C), 139.2 (C), 138.4 (C), 135.9 (C), 134.5 (C), 130.5 (CH × 2), |
| | 128.6 (CH × 2), 128.1 (CH × 3), 127.7 (CH × 2), 126.3 (CH × 2), 117.7 (CH), |
| | 114.4 (C), 114.1 (C), 111.2 (C), 110.2 (CH), 108.5 (CH), 31.5 (CH₂), 19.1 (CH₃). EIMS m/z |
| | (relative intensity) 365 (88), 288 (23), 260 (13), 249 (14), 221 (15), 217 (26), 213 (19), |
| | 158 (27), 131 (30), 111 (100), 91 (68). HRMS Calcd. for C₂₅H₁₉NO₂ 365.1416, found |
| | 365.1415. IR (neat): 3221 (br), 1701, 1560, 1458 cm⁻¹. |
| 230 | ¹H NMR (300 MHz, CDCl₃): δ 8.13-8.08 (m, 2H), 7.67 (d, J = 9.0 Hz, 1H), 7.46 (d, J = 9.0 Hz, |
| | 1H), 7.04-6.99 (m, 2H), 6.30 (d, J = 0.9 Hz, 1H), 3.92 (s, 3H), 2.91 (s, 3H), |
| | 2.52 (d, J = 0.9 Hz, 3H). |
| 231 | ¹H NMR (600 MHz, d6-DMSO): δ 8.30 (d, J = 8.0 Hz, 1H), 7.75 (d, J = 8.6 Hz, 1H), |
| | 7.60 (d, J = 8.0 Hz, 1H), 7.49-7.47 (m, 2H), 7.32-7.30 (m, 1H), 6.28 (q, J = 1.0 Hz, 1H), |
| | 2.49 (d, J = 1.0 Hz, 3H). ¹³C NMR (150 MHz, d6-DMSO): δ 160.2 (C), 155.0 (C), |
| | 149.8 (C), 142.5 (C), 139.5 (C), 126.1 (CH), 122.6 (CH), 122.2 (CH), 120.5 (C), |
| | 120.2 (C), 111.6 (CH), 111.0 (C), 109.9 (CH), 109.4 (C), 108.2 (CH), 18.9 (CH₃). EIMS m/z |
| | (relative intensity) 249 (M⁺, 100), 221 (87), 193 (16), 158 (44), 130 (44), 111 (97), |
| | 91 (95). HRMS Calcd. for C₁₆H₁₁NO₂ 249.0790, found 249.0790. IR (neat): 3250 (br), |
| | 1697, 1630, 1598, 1385, 1336, 1085 cm⁻¹. |
| 232 | ¹H NMR (600 MHz, CDCl₃): δ 8.64 (dd, J = 0.6, 7.7 Hz, 1H), 8.08-8.07 (m, 2H), |
| | 7.68-7.24 (m, 7H), 7.12 (d, J = 8.6 Hz, 1H), 6.22 (q, J = 1.1 Hz, 1H), 5.72 (s, 2H), 2.49 (d, J = 1.1 Hz, |
| | 3H). ¹³C NMR (100 MHz, CDCl₃): δ 192.0 (C), 161.4 (C), 153.8 (C), |
| | 143.3 (C), 140.5 (C), 134.5 (C), 134.4 (CH), 129.2 (CH × 2), 128.5 (CH), 128.1 (CH × 2), |
| | 126.4 (C), 124.1 (CH), 122.0 (CH), 121.5 (C), 121.2 (CH), 113.7 (C), 112.3 (C), 111.2 (CH), |
| | 108.3 (CH), 107.3 (C), 105.2 (CH), 49.1 (CH₂), 19.3 (CH₃). EIMS m/z (relative |
| | intensity) 367 (M⁺, 70), 270 (54), 262 (100), 249 (53), 221 (40), 191 (20), 105 (86), |
| | 77 (23). HRMS Calcd. for C₂₄H₁₇NO₃ 367.1208, found 367.1197. IR (neat): 1718, 1701, |
| | 1630, 1601, 1448, 1390, 1224 cm⁻¹. |
| 233 | ¹H NMR (300 MHz, CDCl₃): δ 7.72 (d, J = 9.0 Hz, 1H), 7.58 (d, J = 9.0 Hz, 1H), |
| | 7.50-7.06 (m, 9H), 6.27 (d, J = 1.2 Hz, 1H), 4.65 (t, J = 2.7 Hz, 2H), 2.54-2.51 (m, 4H). |
| 234 | ¹H NMR (300 MHz, CDCl₃): δ 7.86-7.73 (m, 3H), 7.66-7.55 (m, 2H), 7.47-7.34 (m, |
| | 6H), 6.22 (d, J = 1.2 Hz, 1H), 5.89 (s, 1H), 2.60 (brs, 1H), 2.48 (d, J = 1.2 Hz, 3H). |
| 235 | ¹H NMR (300 MHz, CDCl₃): δ 7.71-7.64 (m, 3H), 7.57-7.45 (m, 4H), 6.26 (d, J = 1.2 Hz, |
| | 1H), 5.18-5.02 (m, 2H), 3.81-3.71 (m, 1H), 2.95-2.67 (m, 2H), 2.50 (d, J = 1.2 Hz, |
| | 3H), 2.39-2.18 (m, 2H). |
| 236 | ¹H NMR (400 MHz, CDCl₃): δ 7.74 (d, J = 6.6 Hz, 1H), 7.59 (d, J = 6.6 Hz, 1H), |
| | 7.49-6.96 (m, 9H), 6.26 (d, J = 0.6 Hz, 1H), 3.93 (t, J = 6.0 Hz, 2H), 3.26 (t, J = 5.1 Hz, 2H), |
| | 2.51 (s, 3H), 2.05-1.85 (m, 4H). |
| 237 | ¹H NMR (300 MHz, CDCl₃): δ 7.74 (d, J = 9.0 Hz, 1H), 7.58 (d, J = 9.0 Hz, 1H), |
| | 7.50-6.96 (m, 9H), 6.26 (d, J = 1.2 Hz, 1H), 4.08 (t, J = 6.6 Hz, 2H), 3.90 (t, J = 6.3 Hz, 2H), |
| | 2.51 (d, J = 0.9 Hz, 3H), 2.05 (s, 3H), 1.82-1.38 (m, 8H). |
| 238 | ¹H NMR (400 MHz, CDCl₃): δ 7.74 (d, J = 6.6 Hz, 1H), 7.57 (d, J = 6.6 Hz, 1H), |
| | 7.50-6.92 (m, 9H), 6.26 (d, J = 0.6 Hz, 1H), 2.51 (d, J = 0.6 Hz, 3H), 0.98 (s, 9H), 0.192 (s, |
| | 6H). |
| 239 | ¹H NMR (300 MHz, d6-DMSO): δ 7.93 (d, J = 9.3 Hz, 1H), 7.76 (d, J = 9.3 Hz, 1H), |
| | 7.72-7.69 (m, 2H), 7.56-7.51 (m, 1H), 7.40-7.35 (m, 2H), 7.28 (d, J = 8.4 Hz, 2H), |
| | 6.51 (d, J = 8.4 Hz, 2H), 6.36 (s, 1H), 2.49 (s, 3H). ¹³C NMR (75 MHz, d6-DMSO): δ |
| | 185.1 (C), 158.9 (C), 157.6 (C), 155.9 (C), 154.0 (C), 149.4 (C), 147.1 (C), 136.6 (C), |
| | 132.9 (CH), 132.1 (CH × 2), 129.3 (CH × 2), 128.2 (CH × 2), 128.1 (C), 125.3 (CH), 119.8 (C), |
| | 115.5 (C), 115.1 (C), 114.4 (CH × 2), 112.7 (CH), 108.9 (CH), 19.0 (CH₃). IR (neat): |
| | 3350 (br), 2957, 2925, 2853, 1731, 1708, 1647, 1601, 1552, 1509, 1472, 1447, 1357, |
| | 1269, 1172, 1081 cm⁻¹. EIMS m/z (relative intensity) 396 (M⁺, 16), 105 (5), 79 (25), |
| | 78 (100), 63 (82). HRMS Calcd. for C₂₅H₁₆O₅ 396.0998, found 396.0998. |
| 240 | ¹H NMR (400 MHz, CDCl₃): δ 7.74 (d, J = 8.8 Hz, 1H), 7.58 (d, J = 8.8 Hz, 1H), |
| | 6.26 (s, 1H), 7.52-7.02 (m, 13H), 4.14 (t, J = 5.6 Hz, 2H), 2.96-2.87 (m, 8H), 2.51 (s, 3H). |
| 241 | ¹H NMR (300 MHz, CDCl₃): δ 7.73 (d, J = 9.0 Hz, 1H), 7.57 (d, J = 9.0 Hz, 1H), |
| | 7.54-7.17 (m, 8H), 7.05 (ddd, J = 8.4, 2.7, 0.9 Hz, 1H), 6.25 (d, J = 1.2 Hz, 1H), 4.13 (t, J = 5.4 Hz, |
| | 2H), 4.06 (s, 4H), 3.16 (t, J = 5.7 Hz, 2H), 2.47 (d, J = 1.2 Hz, 3H). |
| 242 | ¹H NMR (300 MHz, CDCl₃): δ 7.74 (d, J = 9.0 Hz, 1H), 7.58 (d, J = 9.0 Hz, 1H), |
| | 7.50-7.27 (m, 7H), 7.18 (t, J = 8.4 Hz, 1H), 6.98 (dd, J = 8.4, 2.4 Hz, 1H), 6.26 (d, J = 1.2 Hz, |
| | 1H), 3.90 (t, J = 6.6 Hz, 2H), 2.51 (d, J = 1.2 Hz, 3H), 1.75 (quin, J = 6.3 Hz, 2H), |
| | 1.43-1.26 (m, 8H), 0.90 (t, J = 6.9 Hz, 3H). |
| 243 | ¹H NMR (300 MHz, CDCl₃): δ 7.72 (d, J = 8.7 Hz, 1H), 7.56 (d, J = 8.7 Hz, 1H), |
| | 7.50-7.27 (m, 7H), 7.18 (t, J = 8.1 Hz, 1H), 6.98 (ddd, J = 8.1, 2.7, 0.9 Hz, 1H), 6.24 (d, J = 0.9 Hz, |
| | 1H), 3.89 (t, J = 6.6 Hz, 2H), 2.49 (d, J = 0.9 Hz, 3H), 1.75 (quin, J = 6.6 Hz, |
| | 2H), 1.46-1.29 (10H, m), 0.87 (t, J = 6.6 Hz, 3H). |
| 244 | ¹H NMR (300 MHz, CDCl₃): δ 7.74 (d, J = 9.0 Hz, 1H), 7.58 (d, J = 9.0 Hz, 1H), |
| | 7.50-7.27 (m, 7H), 7.18 (t, J = 8.4 Hz, 1H), 6.98 (ddd, J = 8.4, 2.4, 0.9 Hz, 1H), 6.26 (d, J = 1.2 Hz, |
| | 1H), 3.89 (t, J = 6.6 Hz, 2H), 2.51 (d, J = 1.2 Hz, 3H), 1.75 (quin, J = 7.2 Hz, |
| | 1H), 1.50-1.27 (m, 15H), 0.88 (t, J = 6.6 Hz, 3H). |
| 245 | ¹H NMR (300 MHz, CDCl₃): δ 7.76 (d, J = 9.0 Hz, 1H), 7.58 (d, J = 9.0 Hz, 1H), |
| | 7.47-6.79 (m, 8H), 6.28 (d, J = 1.2 Hz, 1H), 3.82 (s, 3H), 2.51 (d, J = 1.2 Hz, 3H). |
| 246 | ¹H NMR (300 MHz, CDCl₃): δ 7.75 (d, J = 9.0 Hz, 1H), 7.58 (d, J = 9.0 Hz, 1H), |
| | 7.44-7.02 (m, 8H), 6.27 (d, J = 1.2 Hz, 1H), 3.80 (s, 3H), 2.51 (d, J = 1.2 Hz, 3H). |
| 247 | ¹H NMR (300 MHz, CDCl₃): δ 7.70 (d, J = 8.7 Hz, 1H), 7.53 (d, J = 8.7 Hz, 1H), |
| | 7.36-7.09 (m, 7H), 6.98-6.94 (m, 1H), 6.22 (d, J = 1.2 Hz, 1H), 3.73 (s, 3H), 2.47 (d, J = 0.9 Hz, |
| | 1H), 2.03 (s, 3H). ¹³C NMR (75 MHz, CDCl₃): δ 185.4, 159.4, 159.1, 156.4, 152.8, |
| | 149.9, 147.9, 137.8, 137.1, 131.5, 129.4, 129.3, 129.0, 128.9, 127.6, 124.2, 122.2, |
| | 119.5, 116.2, 115.2, 113.43, 113.38, 108.8, 55.2, 21.1, 19.4. |
| 248 | ¹H NMR (400 MHz, CDCl₃): δ 7.75-7.69 (m, 3H), 7.54 (d, J = 8.8 Hz, 1H), |
| | 7.46-7.34 (m, 3H), 7.30-7.26 (m, 2H), 6.84-6.80 (m, 2H), 6.24 (q, J = 1.2 Hz, 1H), 3.78 (s, 3H), |
| | 2.48 (d, J = 1.2 Hz, 3H). ¹³C NMR (100 MHz, CDCl₃): δ 185.6 (C), 160.0 (C), |
| | 159.6 (C), 156.5 (C), 152.9 (C), 150.1 (C), 148.0 (C), 136.7 (C), 132.8 (CH), 132.1 (CH × 2), |
| | 129.7 (CH × 2), 128.7 (C), 128.1 (CH × 2), 124.1 (CH), 121.6 (C), 116.4 (C), 115.3 (C), |
| | 113.5 (CH), 113.3 (CH × 2), 109.0 (CH), 55.2 (CH₃), 19.6 (CH₃). |
| | IR (neat): 3058 (w), 2927 (w), 2834 (w), 1730 (s), 1652 (m), 1602 (s), cm⁻¹. |
| | EIMS m/z (relative intensity) 410 (M⁺, 62), 152 (11), 105 (78), 77 (100), 57 (43), |
| | 55 (36). HRMS Calcd. for C₂₆H₁₈O₅ 410.1154, found 410.1153 |
| 249 | ¹H NMR (300 MHz, CDCl₃): δ 7.60-7.00 (m, 11H), 6.11 (s, 1H), 2.40 (d, J = 1.2 Hz, |
| | 3H). |
| 250 | ¹H NMR (300 MHz, CDCl₃): δ 7.74 (d, J = 8.7 Hz, 1H), 7.58 (d, J = 8.7 Hz, 1H), |
| | 7.50-7.16 (m, 13H), 7.00 (dd, J = 8.4, 2.7 Hz, 1H), 6.53 (d, J = 15.9 Hz, 1H), 6.21~5.30 (m, |
| | 2H), 4.05 (t, J = 5.7 Hz, 2H), 3.20-2.96 (m, 2H), 2.81 (t, J = 5.4 Hz, 2H), 2.63-2.47 (m, |
| | 7H). |
| 251 | ¹H NMR (300 MHz, CDCl₃): δ 7.62 (d, J = 9.0 Hz, 1H), 7.360 (d, J = 9.0 Hz, 1H), |
| | 7.312-7.308 (m, 2H), 7.29-7.09 (m, 6H), 6.95-6.92 (m, 1H), 6.08 (s, 1H), 3.86 (t, J = 6.3 Hz, |
| | 2H), 3.72-3.58 (m, 3H), 3.43-3.30 (m, 1H), 2.87-2.79 (m, 1H), 2.68-2.57 (m, 3H), |
| | 2.32 (d, J = 0.9 Hz, 3H), 1.89-1.84 (m, 2H), 1.038 (t, J = 7.2 Hz, 1H). |
| 252 | ¹H NMR (300 MHz, CDCl₃): δ 7.75 (d, J = 9.0 Hz, 1H), 7.58 (d, J = 8.7 Hz, 2H), |
| | 7.50-7.46 (m, 2H), 7.42-7.36 (m, 2H), 7.32-7.24 (m, 2H), 7.09-7.05 (m, 1H), 6.27 (d, J = 1.2 Hz, |
| | 1H), 3.81 (s, 3H), 2.51 (d, J = 0.9 Hz, 3H). |
| 253 | ¹H NMR (300 MHz, CDCl₃): δ 7.77-7.73 (m, 2H), 7.59-7.56 (m, 2H), 7.55-7.45 (m, |
| | 2H), 7.39-7.36 (m, 2H), 7.30-7.26 (m, 2H), 6.27 (d, J = 1.2 Hz, 1H), 3.90 (t, J = 6.6 Hz, |
| | 2H), 2.51 (d, J = 1.2 Hz, 3H), 1.84-1.77 (m, 2H), 1.04 (d, J = 7.5 Hz, 3H). |
| 254 | ¹H NMR (300 MHz, CDCl₃): δ 8.18-8.17 (m, 1H), 7.78-7.75 (m, 2H), 7.63-7.59 (m, |
| | 4H), 7.52-7.49 (m, 1H), 7.21-7.20 (m, 1H), 6.28 (s, 1H), 2.51 (d, J = 0.6 Hz, 3H). |
| 255 | ¹H NMR (300 MHz, CDCl₃): δ 7.74-7.71 (m, 3H), 7.58 (d, J = 9.0 Hz, 1H), |
| | 7.53-7.39 (m, 4H), 6.26 (d, J = 1.2 Hz, 1H), 2.51 (d, J = 1.2 Hz, 3H). LCMS [M + 1]⁺: 277.1. |
| 256 | ¹H NMR (300 MHz, CDCl₃): δ 7.75 (d, J = 8.7 Hz, 1H), 7.58 (d, J = 9.0 Hz, 2H), |
| | 7.56-7.44 (m, 2H), 7.39-7.36 (m, 1H), 7.30-7.22 (m, 3H), 7.05-7.02 (m, 1H), 6.27 (d, J = 1.5 Hz, |
| | 1H), 3.81 (s, 3H), 2.51 (d, J = 1.2 Hz, 3H). ¹³C NMR (75 MHz, CDCl₃): δ 184.9, |
| | 159.3, 159.2, 156.2, 152.8, 149.7, 148.2, 137.6, 133.5, 131.7, 131.6, 129.2, 129.1, |
| | 127.1, 124.5, 122.2, 121.5, 119.7, 115.9, 115.4, 113.6, 113.5, 108.9, 55.3, 19.5. |
| 257 | ¹H NMR (300 MHz, CDCl₃): δ 8.05-8.04 (m, 1H), 7.99-7.63 (m, 2H), 7.58-7.39 (m, |
| | 3H), 7.27-7.12 (m, 2H), 6.24 (s, 1H), 2.48 (s, 3H). |
| 258 | ¹H NMR (300 MHz, CDCl₃): δ 8.19-8.17 (m, 1H), 7.78-7.75 (m, 3H), 7.61-7.55 (m, |
| | 4H), 7.37 (dd, J = 7.5, 7.5 Hz, 1H), 7.21-7.18 (m, 1H), 6.26 (d, J = 1.2 Hz, 1H), 2.51 (d, |
| | J = 1.2 Hz, 3H). ¹³C NMR (75 MHz, CDCl₃): δ 175.0, 159.2, 155.9, 152.6, 149.7, |
| | 147.5, 142.6, 135.3, 134.9, 133.3, 131.9, 131.7, 129.3, 129.0, 128.3, 127.2, 124.5, |
| | 121.7, 116.4, 115.5, 113.8, 108.8, 19.6. |
| 259 | ¹H NMR (300 MHz, CDCl₃): δ 7.73 (d, J = 8.7 Hz, 1H), 7.56-7.48 (m, 2H), |
| | 7.48-7.43 (m, 2H), 7.34-7.17 (m, 3H), 7.01-6.98 (m, 1H), 6.27 (d, J = 0.9 Hz, 1H), 2.50 (d, J = 1.2 Hz, |
| | 3H). ¹³C NMR (75 MHz, CDCl₃): δ 175.0, 159.2, 155.9, 152.6, 149.7, 147.5, |
| | 142.6, 135.3, 134.9, 133.3, 131.9, 131.7, 129.3, 129.0, 128.3, 127.2, 124.5, 121.7, |
| | 116.4, 115.5, 113.8, 108.8, 19.6. |
| 260 | ¹H NMR (300 MHz, CDCl₃): δ 7.73 (d, J = 9.0 Hz, 1H), 7.58-7.54 (m, 2H), |
| | 7.48-7.43 (m, 2H), 7.37-7.34 (m, 1H), 7.27-7.20 (m, 3H), 7.03-6.99 (m, 1H), 6.24 (d, J = 1.2 Hz, |
| | 1H), 4.00 (d, J = 7.2 Hz, 2H), 2.48 (d, J = 1.2 Hz, 3H), 1.40 (d, J = 7.2 Hz, 3H). |
| | ¹³C NMR (75 MHz, CDCl₃): δ 184.9, 159.1, 158.7, 156.2, 152.8, 148.2, 137.6, 133.4, |
| | 131.7, 131.6, 129.2, 129.15, 129.09, 127.0, 124.4, 122.1, 121.5, 120.1, 115.8, 115.4, |
| | 114.2, 113.5, 108.9, 63.6, 19.5, 14.7. |
| 261 | ¹³C NMR (75 MHz, CDCl₃): δ 185.0, 159.2, 158.9, 156.2, 152.8, 149.7, 148.3, 137.6, |
| | 133.5, 131.7, 131.6, 129.2, 129.1, 127.0, 124.4, 122.0, 121.5, 120.1, 115.9, 115.4, |
| | 114.2, 113.6, 108.9, 69.6, 31.5, 19.5, 14.1. |
| 262 | ¹H NMR (300 MHz, CDCl₃): δ 7.74 (d, J = 8.7 Hz, 1H), 7.58-7.56 (m, 2H), |
| | 7.55-7.44 (m, 2H), 7.44-7.34 (m, 1H), 7.27-7.20 (m, 3H), 7.03-7.00 (m, 1H), 6.25 (d, J = 1.2 Hz, |
| | 1H), 3.93 (t, J = 6.6 Hz, 2H), 2.49 (d, J = 1.2 Hz, 3H), 1.78-1.71 (m, 2H), 1.52-1.45 (m, |
| | 2H), 0.98 (d, J = 6.6 Hz, 3H). ¹³C NMR (75 MHz, CDCl₃): δ 185.0, 159.2, 158.9, |
| | 156.3, 152.8, 148.3, 137.6, 133.5, 131.7, 131.6, 129.2, 129.1, 127.0, 124.4, 122.0, |
| | 121.6, 120.2, 115.4, 114.2, 113.6, 108.9, 67.8, 31.1, 19.5, 19.1, 13.8. |
| 263 | ¹H NMR (300 MHz, CDCl₃): δ 7.72 (d, J = 8.7 Hz, 1H), 7.56-7.48 (m, 2H), |
| | 7.46-7.43 (m, 2H), 7.36-7.34 (m, 1H), 7.27-7.19 (m, 3H), 7.03-6.99 (m, 1H), 6.24 (d, J = 1.2 Hz, |
| | 1H), 3.91 (t, J = 6.6 Hz, 2H), 2.48 (s, 3H), 1.79-1.75 (m, 2H), 1.46-1.34 (m, 4H), |
| | 0.93 (d, J = 6.6 Hz, 3H). ¹³C NMR (75 MHz, CDCl₃): δ 185.0, 159.1, 158.9, 156.2, 152.8, |
| | 149.6, 148.2, 137.5, 133.5, 131.7, 131.6, 129.15, 129.08, 127.0, 124.4, 122.0, 121.5, |
| | 120.1, 115.8, 115.4, 114.2, 113.5, 108.9, 68.1, 28.7, 28.0, 22.3, 19.5, 13.9. |
| 264 | ¹H NMR (300 MHz, CDCl₃): δ 7.82-7.79 (m, 2H), 7.68 (d, J = 8.7 Hz, 1H), |
| | 7.57-7.54 (m, 3H), 6.54-6.51 (m, 2H), 6.25 (d, J = 0.9 Hz, 1H), 3.04 (s, 6H), 2.50 (d, J = 1.5 Hz, |
| | 3H). |
| 265 | ¹H NMR (300 MHz, CDCl₃): δ 7.82 (d, J = 9.0 Hz, 2H), 7.72 (d, J = 8.7 Hz, 1H), |
| | 7.57 (d, J = 8.7 Hz, 1H), 7.52-7.33 (m, 5H), 6.80 (d, J = 9.0 Hz, 2H), 6.26 (d, J = 0.9 Hz, |
| | 1H), 4.13 (t, J = 5.6 Hz, 2H), 3.74 (m, 4H), 2.80 (t, J = 5.6 Hz, 2H), 2.57 (m, 4H), |
| | 2.51 (d, J = 0.9 Hz, 3H). |
| 266 | ¹H NMR (300 MHz, CDCl₃): δ 7.81 (d, J = 8.7 Hz, 2H), 7.72 (d, J = 9.0 Hz, 1H), |
| | 7.58 (d, J = 8.7 Hz, 1H), 7.51-7.33 (m, 5H), 6.81 (d, J = 9.3 Hz, 2H), 6.26 (d, J = 0.9 Hz, |
| | 1H), 4.08 (t, J = 5.7 Hz, 2H), 2.73 (t, J = 5.6 Hz, 2H), 2.51 (d, J = 0.9 Hz, 3H), 2.34 (s, |
| | 6H). |
| 267 | ¹H NMR (400 MHz, CDCl₃): δ 7.83 (d, J = 6.6 Hz, 2H), 7.72 (d, J = 6.6 Hz, 1H), |
| | 7.58 (d, J = 6.0 Hz, 1H), 7.51-7.21 (m, 9H), 6.84 (d, J = 6.3 Hz, 2H), 6.26 (s, 1H), 4.20 (t, J = 4.2 Hz, |
| | 2H), 4.07 (s, 4H), 3.19 (t, J = 8.7 Hz, 2H), 2.51 (s, 3H). |

### Example 3

### In Vitro Anti-Influenza Virus Assay (Neutralization Test)

Anti-influenza activities of the coumarin compounds were evaluated by measuring the ability of a test compound to inhibit the cytopathic effect induced by an influenza virus on MDCK cells. The 96-well tissue culture plates were seeded with 200 μL of MDCK cells at a concentration of 1.1×10⁵ cells/mL in DMEM with 10% fetal bovine serum (FBS). The plates were incubated for 24-30 h at 37° C. and were used at about 90% confluency. Influenza A/WSN/33 (H1N1) virus (100 TCID₅₀) was added to the cells and incubated at 35° C. for 1 h. After adsorption, the infected cell plates were overlaid with 50 μL of DMEM plus 2% FBS and a test compound with different concentrations. The plate was incubated at 35° C. for 72 h. At the end of incubation, the plates were fixed by the addition of 100 μL of 4% formaldehyde for 1 h at room temperature. After the removal of formaldehyde, the plates were stained with 0.1% crystal violet for 15 min at room temperature. The plates were washed and dried, and the density of the well was measured at 570 nm. The concentration required for a test compound to reduce the virus-induced cytopathic effect (CPE) by 50% relative to the virus control was expressed as IC₅₀.

Compounds 1-4, 6, 8-12, 14, 16-22, 26, 30-92, 94-98, 100-102, 105-107, 109-122, 127-151, 153-161, 165, 166, 170-191, and 193-263 were tested. Unexpectedly, Compounds 2, 4, 22, 39, 49, 51, 56, 83, 84, 86, 87, 94, 117, 177, 183, 184, 194-199, 216, 217, 224, 231, 243, and 248 showed IC₅₀ values between 6 μM and 25 μM; Compounds 3, 10, 18, 32, 34, 42, 58, 66, 67, 73, 80-82, 97, 116, 133, 136, 147, 149, 153, 154, 161, 165, 171, 178, 181, 182, 185, 187, 190, 193, 201-203, 205, 207, 220, 221, 226, 236, 239-241, 249, 250, 254, and 263 showed IC₅₀ values between 1 μM and 5.9 μM; and Compounds 1, 6, 9, 11, 14, 20, 26, 30, 31, 33, 36, 40, 41, 44-48, 54, 59-61, 68-72, 74-79, 92, 95, 96, 98, 100, 107, 115, 132, 134, 135, 137-146, 148, 150, 155-157, 159, 160, 166, 170, 172, 173, 179, 180, 186, 188, 189, 206, 222, 233, 234, 237, 238, 245-247, 252, and 256-262 showed IC₅₀ values between 10 nM and 0.999 μM.

Compounds 1 and 95 were also tested on various influenza virus strains. Amantadine or Relenza was also tested for comparison. IC₅₀ results are shown in Table 2 below. IC₅₀ is defined as the concentration required for a test compound to reduce the virus-induced cytopathic effect (CPE) by 50% relative to the virus control. Unexpectedly, Compounds 1 and 95 exhibited similar or greater anti-influenza activities, as compared to Amantadine or Relenza.

TABLE 2

| | IC₅₀ (μM) | | | |
|---|---|---|---|---|
| Virus strain | Compound 1 | Compound 95 | Amantadine | Relenza |
| Influenza A/Udorn/72 | 0.347 ± 0.188 | — | 0.980 ± 0.147 | 0.670 ± 0.232 |
| (H3N2) | | | | |
| Influenza A/TW/83/05 | 0.057 ± 0.006 | — | >25 | 0.918 ± 0.026 |
| (H3N2) | | | | |
| Influenza A/TW/3446/04 | — | 0.15 ± 0.01 | — | 0.03 ± 0.01 |
| (H3N2) | | | | |
| Influenza A/TW/785/05 | 0.052 ± 0.001 | — | 0.382 ± 0.238 | 1.056 ± 0.982 |
| (H1N1) | | | | |
| Influenza A/TW/141/04 | — | 0.09 ± 0.01 | — | 0.35 ± 0.14 |
| (H1N1) | | | | |
| Influenza A/WSN/33 (H1N1) | 0.119 ± 0.043 | 0.07 ± 0.01 | >25 | 0.062 ± 0.018 |
| Influenza B/TW/710/05 | 0.102 ± 0.020 | 0.09 ± 0.01 | >25 | 0.030 ± 0.019 |
| Influenza B/TW/70325/05 | 0.067 ± 0.002 | 0.09 ± 0.01 | >25 | 0.020 ± 0.010 |
| Influenza B/TW/99/07 | 0.089 ± 0.014 | 0.04 ± 0.01 | >25 | 0.099 ± 0.009 |

### Example 4

### In Vitro EV 71, Coxsackie Virus B3, and Human Rhinovirus 2 Neutralization Assay

This assay measured the ability of a test compound to inhibit the cytopathic effect induced by a picornavirus (EV71, Coxsackie Virus B3, or human rhinovirus 2) on R_{D} cells. The method used for this assay is described in Chang et al., J Med Chem, 2005, 48(10), 3522-3535. More specifically, 96-well tissue culture plates were seeded with 200 μL of R_{D} cells at a concentration of 3×10⁵ cells/mL in DMEM with 10% FBS. The plates were incubated for 24-30 h at 37° C. and were used at about 90% confluency. Virus (100 TCID50) mixed with different concentrations of a test compound was added to the cells and incubated at 37° C. for 1 h. After adsorption, the infected cell plates were overlaid with 50 μL of DMEM plus 5% FBS and 2% DMSO. The plate was wrapped in Parafilm and incubated at 37° C. for 64 h. At the end of incubation, the plates were fixed by the addition of 100 μL of 0.5% glutaraldehyde for 1 h at room temperature. After the removal of glutaraldehyde, the plates were stained with 0.1% crystal violet for 15 min at room temperature. The plates were washed and dried, and the density of the well was measured at 570 nm. The concentration required for a test compound to reduce the virus-induced cytopathic effect (CPE) by 50% relative to the virus control is expressed as IC₅₀.

Compound 1 was tested. Amantadine and Relenza were also tested for comparison. Results are shown in Table 3 below. Unexpectedly, Compound 1 exhibited much greater inhibition of cytopathic effect induced by picornaviruses, as compared to Amantadine or Relenza.

TABLE 3

| | IC₅₀ (μM) | | |
|---|---|---|---|
| Virus | Compound 1 | Amantadine | Relenza |
| Enterovirus 71 (4643) | 0.002 ± 0.001 | >25 | >25 |
| Coxsackie B virus 3 | 0.005 ± 0.001 | >25 | >25 |
| Human rhinovirus 2 | 0.012 ± 0.001 | >25 | >25 |

### Example 5

### In Vitro HSV-1 Plaque Reduction Assay

The method used for this assay is described in Su et al., Antiviral Res., 2008, 79(1), 62-70.

Vero cells were seeded onto a 96-well culture plate at a concentration of 10⁴ cells per well one day before infection. Next day, medium was removed and 10 plaque forming unit (pfu) HSV-1 suspension per well were added and incubated at 37° C. with 5% CO₂ for 1 h. The infected cell monolayer was then washed with phosphate buffered saline (PBS) and cultured in maintenance medium containing 1 μM of compounds. After 72 h of incubation at 37° C., cell monolayer was fixed with 10% formalin and stained with 1% crystal violet. Compounds protecting more than 50% of cells from lysis by HSV infection were considered to possess antiviral activity and were further analyzed.

Plaque assays were performed with monolayer cultures of Vero cells in 24-well culture plates. For plaque reduction assay, cell monolayer was infected with virus (50 pfu/well) and incubated at 37° C. with 5% CO₂ for 1 h. The infected cell monolayer was then washed three times with PBS and overlaid with overlapping solution (maintenance medium containing 1% methylcellulose and various concentrations of indicated compounds). After 72 h of incubation at 37° C., cell monolayer was fixed with 10% formalin and stained with 1% crystal violet. Plaques were counted and the percentage of inhibition was calculated as [100−(V_{D}/V_{C})]×100%, where V_{D} and V_{C} refer to the virus titer in the presence and absence of the compound, respectively. The minimal concentration of compounds required to reduce 50% of plaque numbers (EC₅₀) was calculated by regression analysis of the dose-response curves generated from plaque assays.

Compound 1 was tested and unexpectedly showed an EC₅₀ value of about 0.5 μM.

### Example 6

### In Vitro EBV Assay

The method used for this assay is described in Chang et al., J Virol, 1999, 73, 8857-8866 and Tsai et al., J Virol Methods, 1991, 33, 47-52.

To suppress EBV reactivation, a test compound was added to the NA cell culture medium at indicated final concentration 24 h prior to 12-o-tetradecanoylphorbol-13-acetate (TPA)/sodium n-butyrate (SB) treatment. After treatment, the cells were fixed and assayed by anti-EBV-EAD immunofluorescence for detection of EBV reactivation. The treatment with the test compound inhibited EBV reactivation in NPC cells. NA cells were subjected to treatment with the test compound 24 h prior to the addition of TPA/SB. EBV reactivation was significantly suppressed when compared to the mock-treated (0 μM) cells. Cells were stained with anti-EBV EAD antibody. The location of cell nuclei in the same fields was revealed by staining with Hoechst 33258. The minimal concentration of the test compound required to reduce 50% of virus replication numbers (EC₅₀) was calculated from regression analysis of the dose-response curves obtained from anti-EBV-EAD immunofluorescence.

Compound 1 was tested and unexpectedly showed an EC₅₀ value of less than 0.5 μM.

### Example 7

### Inhibition of HIV-1 Replication in Peripheral Blood Mononuclear Cells

Equal amounts of wild-type and mutant viruses were used to infect 5×10⁴ peripheral blood mononuclear cells (PBMC) in 1.5 mL medium containing DMSO or various concentrations of a test compound. A half-milliliter of the culture medium was collected from each culture at days 3, 5, and 7. Viral RNA was extracted from the collected culture supernatants and the viral titers (copies/mL) were determined by real-time PCR. The percentage of inhibition was calculated as [100−(V_{D}/V_{C})]×100%, where V_{D} and V_{C} refer to the virus titers in the presence and absence of the test compound, respectively.

Compounds 1, 33, 95, 134, 140, and 141 were tested in this assay. Unexpectedly, they all showed inhibition of HIV replication. AZT (also known as zidovudine) was also tested for comparison. Results are shown in Table 4 below.

TABLE 4

| Compound | Inhibition (%) | | |
|---|---|---|---|
| (concentration)^{a} | Day 3 | Day 5 | Day 7 |

| | | | | |
|---|---|---|---|---|
| 1 | (0.1 μM) | 2 | 82 | 91 |
| 33 | (0.1 μM) | 5 | 98 | 99.5 |
| 95 | (0.1 μM) | 42 | 89 | 99 |
| 134 | (0.1 μM) | 61 | 91 | 96 |
| 140 | (0.1 μM) | 19 | 65 | 93 |
| 141 | (0.1 μM) | 19 | 71 | 97 |
| AZT | (0.05 μM) | 6 | 71 | 88 |

^{a}All tested compounds showed CC₅₀ values (50% cytotoxicity concentration) higher than 1.25 μM.

### Example 8

### In Vitro Assessment of Anti-HSV Activity

Anti-HSV activities of compounds described herein were evaluated by performing a plaque reduction assay using monolayer cultures of Vero cells in 24-well culture plates. A cell monolayer was infected with herpes simplex virus type 1 (50 pfu/well) and incubated at 37° C. with 5% CO₂ for 1 h. The infected cell monolayer was then washed three times with PBS and overlaid with a solution (maintenance medium containing 1% methylcellulose and various concentrations of a test compound). After 72 h of incubation at 37° C., the cell monolayer was fixed with 10% formalin and stained with 1% crystal violet. Plaques were counted and the percentage of inhibition was calculated as [100−(V_{D}/V_{C})]×100%, where V_{D} and V_{C} refer to the virus titer in the presence and absence of the compound, respectively. The minimal concentration of a compound required to reduce 50% of plaque numbers (EC₅₀) was calculated by regression analysis of the dose-response curves generated from the plaque assay.

Compounds 33, 95, 134, 140, and 141 were tested in this assay. Results are shown in Table 5 below.

TABLE 5

| Compound | EC₅₀ (μM)^{b} | CC₅₀ (μM)^{c} | SI^{d} |
|---|---|---|---|
| 33 | 0.396 | 93.44 | 235.972 |
| 95 | 0.0600 | >100 | >1666.666 |
| 134 | 0.0323 | >100 | >3095.975 |
| 140 | 0.343 | >100 | >291.545 |
| 141 | 0.00390 | >100 | >25641.03 |

^{b}50% effective concentration
^{c}50% cytotoxicity concentration
^{d}selectivity index = CC₅₀/EC₅₀

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A method for treating an infection with a virus, comprising administering to a subject in need thereof an effective amount of a compound of formula (I): 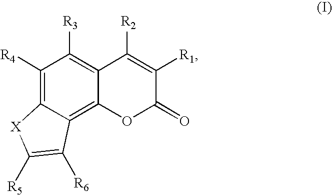
wherein
each of R₁, R₂, R₃, and R₄, independently, is H, alkyl, alkenyl, alkynyl, cycloalkyl, cycloalkenyl, heterocycloalkyl, heterocycloalkenyl, aryl, heteroaryl, halo, nitro, cyano, amino, hydroxy, alkoxy, aryloxy, C(O)Rₐ, C(O)O, C(O)NRₐR_{b}, C(S)R_{b}, or C(NR_{b})Rₐ, in which each of Rₐ and R_{b}, independently, is H, alkyl, alkenyl, alkynyl, hydroxy, alkoxy, amino, cycloalkyl, cycloalkenyl, heterocycloalkyl, heterocycloalkenyl, aryl, or heteroaryl; or R₁ and R₂, together with the carbon atoms to which they are bonded, are cycloalkenyl, heterocycloalkenyl, aryl, or heteroaryl; or R₂ and R₃, together with the carbon atoms to which they are bonded, are cycloalkenyl or heterocycloalkenyl; or R₃ and R₄, together with the carbon atoms to which they are bonded, are cycloalkenyl, heterocycloalkenyl, aryl, or heteroaryl;
R₅ is alkyl substituted with aryl or hydroxy, alkenyl, alkynyl, cycloalkyl, cycloalkenyl, heterocycloalkyl, heterocycloalkenyl, aryl, heteroaryl, halo, nitro, cyano, amino, hydroxy, alkoxy, aryloxy, C(O)R_{c}, C(O)OR_{c}, C(O)NR_{c}R_{d}, C(S)R_{d}, or C(NR_{d})R_{c}, in which each of R_{c} and R_{d}, independently, is H, alkyl, alkenyl, alkynyl, hydroxy, alkoxy, amino, cycloalkyl, cycloalkenyl, heterocycloalkyl, heterocycloalkenyl, aryl, or heteroaryl;
R₆ is H, alkyl, alkenyl, alkynyl, cycloalkyl, cycloalkenyl, heterocycloalkyl, heterocycloalkenyl, aryl, heteroaryl, halo, nitro, cyano, amino, hydroxy, alkoxy, aryloxy, C(O)R_{c}, C(O)OR_{c}, C(O)NR_{c}R_{d}, C(S)R_{d}, or C(NR_{d})R_{c}; or R₅ and R₆, together with the carbon atoms to which they are bonded, are cycloalkenyl, heterocycloalkenyl, aryl, or heteroaryl; and
X is O, S, or N(Rₑ), in which Rₑ is H, alkyl, cycloalkyl, heterocycloalkyl, aryl, or heteroaryl.

2. The method of claim 1, wherein the virus is influenza virus, human rhinovirus 2, Herpes simplex virus, enterovirus 71, Coxsackie Virus B3, Hepatitis C virus, Hepatitis B virus, Epstein-Barr virus, or Human Immunodeficiency Virus.

3. The method of claim 2, wherein the virus is influenza virus.

4. The method of claim 1, wherein R₅ is alkyl substituted with aryl or hydroxy, cycloalkyl, aryl, halo, C(O)R_{c}, or C(O)OR_{c}.

5. The method of claim 4, wherein R₅ is alkyl substituted with aryl or hydroxy, or C(O)R_{c}, in which R_{c} is aryl or heteroaryl.

6. The method of claim 5, wherein R₆ is alkyl, cycloalkyl, aryl, or heteroaryl.

7. The method of claim 6, wherein R₆ is aryl or heteroaryl.

8. The method of claim 7, wherein X is O and R₂ is alkyl.

9. The method of claim 1, wherein R₆ is alkyl, cycloalkyl, aryl, or heteroaryl.

10. The method of claim 9, wherein R₆ is aryl or heteroaryl.

11. The method of claim 1, wherein X is O.

12. The method of claim 1, wherein each of R₁, R₂, R₃, and R₄, independently, is H, alkyl, aryl, heteroaryl, nitro, hydroxy, alkoxy, aryloxy, or C(O)Rₐ; or R₁ and R₂, together with the carbon atoms to which they are bonded, are cycloalkenyl, heterocycloalkenyl, aryl, or heteroaryl.

13. The method of claim 1, wherein R₅ is C(S)R_{d} or C(NR_{d})R_{c}.

14. The method of claim 13, wherein R₆ is aryl or heteroaryl.

15. The method of claim 14, wherein X is O and R₂ is alkyl.

16. The method of claim 1, wherein the compound is one of Compounds 1, 6, 9, 11, 14, 20, 26, 30, 31, 33, 36, 40, 41, 44-48, 54, 59-61, 68-72, 74-79, 92, 95, 96, 98, 100, 107, 115, 132, 134, 135, 137-146, 148, 150, 155-157, 159, 160, 166, 170, 172, 173, 179, 180, 186, 188, 189, 206, 222, 233, 234, 237, 238, 245-247, 252, and 256-262.

17. A pharmaceutical composition comprising a pharmaceutically acceptable carrier and a compound of formula (I): 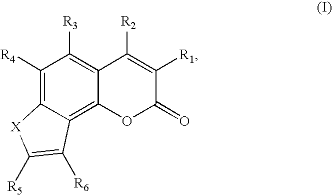
wherein
each of R₁, R₂, R₃, and R₄, independently, is H, alkyl, alkenyl, alkynyl, cycloalkyl, cycloalkenyl, heterocycloalkyl, heterocycloalkenyl, aryl, heteroaryl, halo, nitro, cyano, amino, hydroxy, alkoxy, aryloxy, C(O)Rₐ, C(O)ORₐ, C(O)NₐR_{b}, C(S)R_{b}, or C(NR_{b})Rₐ, in which each of Rₐ and R_{b}, independently, is H, alkyl, alkenyl, alkynyl, hydroxy, alkoxy, amino, cycloalkyl, cycloalkenyl, heterocycloalkyl, heterocycloalkenyl, aryl, or heteroaryl; or R₁ and R₂, together with the carbon atoms to which they are bonded, are cycloalkenyl, heterocycloalkenyl, aryl, or heteroaryl; or R₂ and R₃, together with the carbon atoms to which they are bonded, are cycloalkenyl or heterocycloalkenyl; or R₃ and R₄, together with the carbon atoms to which they are bonded, are cycloalkenyl, heterocycloalkenyl, aryl, or heteroaryl;
R₅ is alkyl substituted with aryl or hydroxy, alkenyl, alkynyl, cycloalkyl, cycloalkenyl, heterocycloalkyl, heterocycloalkenyl, aryl, heteroaryl, halo, nitro, cyano, amino, hydroxy, alkoxy, aryloxy, C(O)R_{c}, C(O)OR_{c}, C(O)NR_{c}R_{d}, C(S)R_{d}, or C(NR_{d})R_{c}, in which each of R_{c} and R_{d}, independently, is H, alkyl, alkenyl, alkynyl, hydroxy, alkoxy, amino, cycloalkyl, cycloalkenyl, heterocycloalkyl, heterocycloalkenyl, aryl, or heteroaryl;
R₆ is alkenyl, alkynyl, cycloalkyl, cycloalkenyl, heterocycloalkyl, heterocycloalkenyl, heteroaryl, halo, nitro, cyano, amino, hydroxy, alkoxy, aryloxy, C(O)R_{c}, C(O)OR_{c}, C(O)NR_{c}R_{d}, C(S)R_{d}, C(NR_{d})R_{c}, or aryl substituted with alkyl at the 3-position of the aryl, halo, nitro, cyano, amino, cycloalkyl, aryl, or heteroaryl; and
X is O, S, or N(Rₑ), in which Rₑ is H, alkyl, cycloalkyl, heterocycloalkyl, aryl, or heteroaryl.

18. The composition of claim 17, wherein R₅ is alkyl substituted with aryl or hydroxy, cycloalkyl, aryl, halo, C(O)R_{c}, C(O)OR_{c}, C(O)NR_{c}R_{d}, C(S)R_{d}, or C(NR_{d})R_{c}.

19. The composition of claim 18, wherein R₅ is C(O)R_{c} or C(O)OR_{c}, in which R_{c} is aryl or heteroaryl.

20. The composition of claim 19, wherein R₆ is cycloalkyl, heteroaryl, or aryl substituted with alkyl at the 3-position of the aryl, halo, nitro, cyano, amino, cycloalkyl, aryl, or heteroaryl.

21. The composition of claim 20, wherein R₆ is heteroaryl or phenyl substituted with alkyl at the 3-position of the phenyl, halo, nitro, cyano, or amino.

22. The composition of claim 21, wherein X is O and R₂ is alkyl or C(O)H.

23. The composition of claim 22, wherein the compound is one of Compounds 132, 134, 135, 137-141, 143-148, 150, 151, 159, 160, 207, 222, and 234.

24. The composition of claim 17, wherein X is O and each of R₁, R₂, R₃, and R₄, independently, is H, alkyl, alkenyl, cycloalkyl, cycloalkenyl, heterocycloalkyl, aryl, hydroxy, alkoxy, halo, cyano, nitro, or C(O)H.

25. A pharmaceutical composition comprising a pharmaceutically acceptable carrier and a compound of formula (I): 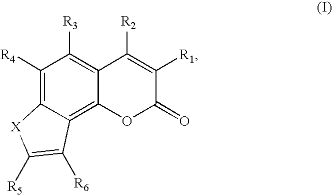
wherein
each of R₁, R₂, R₃, and R₄, independently, is H, alkyl, alkenyl, alkynyl, cycloalkyl, cycloalkenyl, heterocycloalkyl, heterocycloalkenyl, aryl, heteroaryl, halo, nitro, cyano, amino, hydroxy, alkoxy, aryloxy, C(O)Rₐ, C(O)ORₐ, C(O)NRₐR_{b}, C(S)R_{b}, or C(NR_{b})Rₐ, in which each of Rₐ and R_{b}, independently, is H, alkyl, alkenyl, alkynyl, hydroxy, alkoxy, amino, cycloalkyl, cycloalkenyl, heterocycloalkyl, heterocycloalkenyl, aryl, or heteroaryl; or R₁ and R₂, together with the carbon atoms to which they are bonded, are cycloalkenyl, heterocycloalkenyl, aryl, or heteroaryl; or R₂ and R₃, together with the carbon atoms to which they are bonded, are cycloalkenyl or heterocycloalkenyl; or R₃ and R₄, together with the carbon atoms to which they are bonded, are cycloalkenyl, heterocycloalkenyl, aryl, or heteroaryl;
R₅ is alkyl substituted with aryl or hydroxy, alkenyl, alkynyl, cycloalkyl, cycloalkenyl, heterocycloalkyl, heterocycloalkenyl, aryl, heteroaryl, halo, nitro, cyano, amino, hydroxy, alkoxy, aryloxy, C(O)R_{c}, C(O)OR_{c}, C(O)NR_{d}Rₑ, C(S)R_{d}, or C(NRₑ)R_{d}, in which R_{c} is cycloalkyl, cycloalkenyl, heterocycloalkyl, heterocycloalkenyl, heteroaryl, or aryl substituted with alkyl, halo, nitro, cyano, amino, amido, cycloalkyl, aryl, heteroaryl, hydroxy, alkoxy, acyloxy, silyloxy, or phosphate at the 2- or 3-position of the aryl, and each of R_{d} and Rₑ, independently, is H, alkyl, alkenyl, alkynyl, hydroxy, alkoxy, amino, cycloalkyl, cycloalkenyl, heterocycloalkyl, heterocycloalkenyl, aryl, or heteroaryl;
R₆ is alkyl, alkynyl, cycloalkyl, cycloalkenyl, heterocycloalkyl, heterocycloalkenyl, aryl, heteroaryl, halo, nitro, cyano, amino, hydroxy, alkoxy, or aryloxy; and
X is O, S, or N(R_{f}), in which R_{f} is H, alkyl, cycloalkyl, heterocycloalkyl, aryl, or heteroaryl.

26. The composition of claim 25, wherein R₆ is heteroaryl or aryl.

27. The composition of claim 26, wherein R₅ is C(O)R_{c}C or C(O)OR_{c}, in which R_{c} is heteroaryl or aryl substituted with alkyl, halo, nitro, cyano, amino, amido, cycloalkyl, aryl, heteroaryl, hydroxy, alkoxy, acyloxy, silyloxy, or phosphate at the 2- or 3-position of the aryl.

28. The composition of claim 27, wherein the compound is one of Compounds 30, 31, 33, 36, 39, 40, 44, 45, 47,48,56,57,59-61, 67-92, 95, 96, 98, 100, 102, 107, 115, 177-191, 193-203, 233, 236-243, 245-247, 249, 250, 252, and 256-263.

29. The composition of claim 26, wherein R₅ is alkyl substituted with aryl or hydroxy, C(S)R_{d}, or C(NRₑ)R_{d}.

30. The composition of claim 28, wherein the compound is one of Compounds 166 and 170-173.

31. The composition of claim 25, wherein X is O and each of R₁, R₂, R₃, and R₄, independently, is H, alkyl, alkenyl, cycloalkyl, cycloalkenyl, heterocycloalkyl, aryl, hydroxy, alkoxy, halo, cyano, nitro, or C(O)H.

32. A pharmaceutical composition comprising a pharmaceutically acceptable carrier and a compound of formula (I): 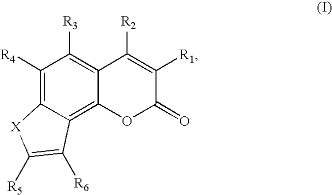
wherein
each of R₁, R₃, and R₄, independently, is H, alkyl, alkenyl, alkynyl, cycloalkyl, cycloalkenyl, heterocycloalkyl, heterocycloalkenyl, aryl, heteroaryl, halo, nitro, cyano, amino, hydroxy, alkoxy, aryloxy, C(O)Rₐ, C(O)ORₐ, C(O)NₐR_{b}, C(S)R_{b}, or C(NR_{b})Rₐ, in which each of Rₐ and R_{b}, independently, is H, alkyl, alkenyl, alkynyl, hydroxy, alkoxy, amino, cycloalkyl, cycloalkenyl, heterocycloalkyl, heterocycloalkenyl, aryl, or heteroaryl; R₂ is H, C₂-C₁₀ alkyl, alkenyl, alkynyl, cycloalkyl, cycloalkenyl, heterocycloalkyl, heterocycloalkenyl, heteroaryl, halo, nitro, cyano, amino, hydroxy, alkoxy, aryloxy, C(O)Rₐ, C(O)ORₐ, C(O)NRₐR_{b}, C(S)R_{b}, or C(NR_{b})Rₐ; or R₁ and R₂, together with the carbon atoms to which they are bonded, are cycloalkenyl, heterocycloalkenyl, aryl, or heteroaryl; or R₂ and R₃, together with the carbon atoms to which they are bonded, are cycloalkenyl or heterocycloalkenyl; or R₃ and R₄, together with the carbon atoms to which they are bonded, are cycloalkenyl, heterocycloalkenyl, aryl, or heteroaryl;
R₅ is C(O)R_{c}, C(O)OR_{c}, C(O)NR_{d}Rₑ, C(S)R_{d}, or C(NRₑ)R_{d}, in which, R_{c} is aryl or heteroaryl, and each of R_{d} and Rₑ, independently, is H, alkyl, alkenyl, alkynyl, hydroxy, alkoxy, amino, cycloalkyl, cycloalkenyl, heterocycloalkyl, heterocycloalkenyl, aryl, or heteroaryl;
R₆ is aryl or heteroaryl; and
X is O, S, or N(R_{f}), in which R_{f} is H, alkyl, cycloalkyl, heterocycloalkyl, aryl, or heteroaryl.

33. The composition of claim 32, wherein X is O and R₂ is C₂-C₁₀ alkyl or C(O)H.

34. The composition of claim 33, wherein the compound is Compound 9, 10, 14, or 20.

35. The composition of claim 32, wherein X is O and each of R₁, R₃, and R₄, independently, is H, alkyl, alkenyl, cycloalkyl, cycloalkenyl, heterocycloalkyl, aryl, hydroxy, alkoxy, halo, cyano, nitro, or C(O)H.

36. A pharmaceutical composition comprising a pharmaceutically acceptable carrier and a compound, wherein the compound is one of Compounds 1, 4, 6, 11, 26, 32, 34, 41, 46, 53-55, 155-157, and 206.
